(19) United States
(12) Patent Application Publication
(10) Pub. No.: US 2025/0049355 A1
(43) Pub. Date: Feb. 13, 2025

(54) SYSTEMS AND METHODS FOR PROVIDING THERAPY MANAGEMENT RECOMMENDATIONS FOR DIABETIC PATIENTS AND PATIENTS WITH LIVER DISEASE

(71) Applicant: Dexcom, Inc., San Diego, CA (US)

(72) Inventors: Qi AN, San Diego, CA (US); Matthew L. JOHNSON, San Diego, CA (US); John PADERI, San Diego, CA (US); Rush BARTLETT, San Diego, CA (US)

(21) Appl. No.: 18/792,478

(22) Filed: Aug. 1, 2024

Related U.S. Application Data
(63) Continuation of application No. 63/517,076, filed on Aug. 1, 2023.

(51) Int. Cl. A61B 5/145; A61B 5/00; G16H 20/00

(52) U.S. Cl. CPC A61B 5/14546; A61B 5/14532; A61B 5/4244; A61B 5/6847; A61B 5/7267; G16H 20/00

(57) ABSTRACT

Certain aspects of the present disclosure provide a monitoring system comprising a continuous analyte sensor configured to penetrate a skin of a patient and generate a sensor current indicative of analyte levels of the patient, and a sensor electronics module coupled to the continuous analyte sensor. The sensor electronics module comprises an analog to digital converter configured to receive the sensor and convert the sensor current generated by the continuous analyte sensor into digital signals, one or more processors configured to convert the digital signals to a set of analyte measurements indicative of the analyte levels of the patient, and a Bluetooth antenna configured to transmit the set of analyte measurements wirelessly to a wireless communications device using Bluetooth or BLE communications protocols.

### CROSS-REFERENCE TO RELATED APPLICATIONS

This application claims priority to and benefit of U.S. Provisional Application No. 63/517,076, filed Aug. 1, 2023, which is hereby assigned to the assignee hereof and hereby expressly incorporated by reference in their entirety as if fully set forth below and for all applicable purposes.

### BACKGROUND

Generally, a final confirmatory diagnosis of liver disease requires a biopsy. However, although biopsies are the gold standard for confirmatory diagnosis of liver disease, they are not widely used due to the invasiveness of the procedure, which may cause liver disease to be generally undiagnosed until patients develop signs of severe liver disease.

Further, although some non-invasive screening tools, such as Fibroscan (an imaging based estimate of liver stiffness) or metabolic assay panels (a blood test to test various liver enzymes), have recently become available, these tools are not always as reliable and/or accurate in confirming a liver disease diagnosis.

In addition, the biopsies and screening tools discussed above are point-in-time diagnostic methods and do not provide insight into the health and functioning of the liver over time.

Consequently, there is a need in the art for an accurate, non-invasive solution for confirming a liver disease diagnosis and monitoring the health and functioning of the liver over time.

### BRIEF DESCRIPTION OF THE DRAWINGS

So that the manner in which the above-recited features of the present disclosure can be understood in detail, a more particular description, briefly summarized above, may be had by reference to aspects, some of which are illustrated in the drawings. It is to be noted, however, that the appended drawings illustrate only certain typical aspects of this disclosure and are therefore not to be considered limiting of its scope, for the description may admit to other equally effective aspects.

FIG. 1 illustrates aspects of an example therapy management system used in connection with implementing embodiments of the present disclosure.

FIG. 2 is a diagram conceptually illustrating an example continuous analyte monitoring system including example continuous analyte sensor(s) with sensor electronics, according to certain embodiments of the present disclosure.

FIG. 3 illustrates example inputs and example metrics that are calculated based on the inputs for use by the therapy management system of FIG. 1, according to certain embodiments of the present disclosure.

FIG. 4 describes an example method for classifying a patient, and providing therapy management recommendations using an analyte monitoring system configured to measure at least glucose levels, according to certain embodiments of the present disclosure.

FIG. 5 describes an example method for monitoring progression of liver disease and/or diabetes based on therapy management recommendations, according to certain embodiments of the present disclosure.

FIG. 6 describes an example method for determining a patient's liver disease risk and providing therapy management recommendations to reduce the patient's liver disease risk and/or prevent liver disease development, according to certain embodiments of the present disclosure.

FIG. 7 is a flow diagram depicting a method for training machine learning models to predict a patient's disease state and provide therapy management recommendations to a patient based on the disease state, according to certain embodiments of the present disclosure.

FIG. 8 is a block diagram depicting a computing device configured to perform the operations of FIG. 4, according to certain embodiments of the present disclosure.

FIGS. 9A-9B depict exemplary enzyme domain configurations for a continuous multi-analyte sensor, according to certain embodiments of the present disclosure.

FIGS. 9C-9D depict exemplary enzyme domain configurations for a continuous multi-analyte sensor, according to certain embodiments of the present disclosure.

FIG. 9E depicts an exemplary enzyme domain configuration for a continuous multi-analyte sensor, according to certain embodiments of the present disclosure.

FIGS. 10A-10B depict alternative views of an exemplary dual electrode enzyme domain configuration for a continuous multi-analyte sensor, according to certain embodiments of the present disclosure.

FIGS. 10C-10D depict alternative views of an exemplary dual electrode enzyme domain configuration for a continuous multi-analyte sensor, according to certain embodiments of the present disclosure.

FIG. 10E depicts an exemplary dual electrode configuration for a continuous multi-analyte sensor, according to certain embodiments of the present disclosure.

FIG. 11A depicts an exemplary enzyme domain configuration for a continuous multi-analyte sensor, according to certain embodiments of the present disclosure.

FIGS. 11B-11C depict alternative exemplary enzyme domain configurations for a continuous multi-analyte sensor, according to certain embodiments of the present disclosure.

FIG. 12 depicts an exemplary enzyme domain configuration for a continuous multi-analyte sensor, according to certain embodiments of the present disclosure.

FIGS. 13A-13D depict alternative views of exemplary dual electrode enzyme domain configurations G1-G4 for a continuous multi-analyte sensor, according to certain embodiments of the present disclosure.

To facilitate understanding, identical reference numerals have been used, where possible, to designate identical elements that are common to the figures. It is contemplated that elements disclosed in one aspect may be beneficially utilized on other aspects without specific recitation.

### DETAILED DESCRIPTION

The present disclosure relates generally to methods and systems for continuously monitoring analyte data, including at least glucose data, and/or non-analyte data to: (1) monitor for the occurrence or development of liver disease in a healthy patient, or monitor liver function, (2) monitor for progression of liver disease (e.g., worsening stage of liver disease) and/or development of diabetes in a patient with liver disease, and (3) determine if a diabetic patient has liver disease, whether the patient is at risk of developing liver disease, or whether the patient's liver function is worsening. For example, aspects of the present disclosure utilize glucose metrics as well as other analyte or non-analyte data of a patient to determine a risk or presence of liver disease or diabetes, determine the progression of liver disease or diabetes, and provide patient-specific therapy management recommendations (e.g., meal, exercise, liver disease stage information, and/or lifestyle recommendations) to prevent the progression and/or development of liver disease.

Determining risk of liver disease and/or diagnosing liver disease earlier than otherwise possible with a biopsy, allows for better outcomes for patients with liver disease. In certain embodiments herein, glucose data and glucose metrics can be utilized by non-invasive methods to detect the risk and presence of liver disease earlier and more accurately. When the risk or presence of liver disease is identified early, clinicians and caregivers may provide treatment recommendations to prevent the disease or the progression thereof. In some examples, treatment recommendations may include prescription medications, lifestyle changes, meal recommendations and/or exercise recommendations that may prevent liver disease, cause regression of liver disease, and/or prevent progression of liver disease.

As used herein, the term “continuous” analyte monitoring refers to monitoring one or more analytes in a fully continuous, semi-continuous, or periodic manner, which results in a data stream of analyte values over time without requiring user intervention (e.g., repeated finger sticks). A data stream of analyte values over time is what allows for meaningful data and insight to be derived using the algorithms described herein for determining a risk or presence of liver disease or diabetes, determining the progression of liver disease or diabetes, and providing patient-specific therapy management guidance (e.g., meal, exercise, liver disease stage information, and/or lifestyle recommendations) to prevent the progression and/or development of liver disease. In other words, single point-in-time measurements collected as a result of a patient visiting their health care professional every few months, or using point of care meters, results in sporadic data points (e.g., that are, at best, hours, days or months apart in timing) that cannot form the basis of any meaningful data or insight to be derived. As such, without the continuous analyte monitoring system of the embodiments herein, it is simply impossible to determine through pattern analysis and continuously a risk or presence of liver disease or diabetes, determine the progression of liver disease or diabetes, as well as continuously provide therapy management recommendations (e.g., meal, exercise, liver disease stage information, and/or lifestyle recommendations) to prevent the progression and/or development of liver disease, as described herein.

The data stream of analyte values collected over time, with the continuous analyte monitoring system presented herein, include real-time analyte values (values representative of the current concentration of analyte values in the body), which allows for deriving meaningful data and insight in real-time using the systems and algorithms described herein. The derived real-time data and insight in turn allows for assessing the risk of disglycemia due to liver disease or diabetes that may indicate the presence of such disease and/or progression of such disease, as well as real-time therapy management recommendations. Real time analyte values herein refer to analyte values that become available and actionable within seconds or minutes of being produced as a result of at least one sensor electronics module of the continuous analyte monitoring system (1) converting sensor current(s) (i.e., analog electrical signals) generated by the continuous analyte sensor(s) into sensor count values, (2) processing the count values (e.g., by filtering, smoothing, calibration or some combination thereof) to generate at least glucose concentration values using calibration techniques described herein to account for the sensitivity of the continuous analyte sensor(s), and (3) transmitting measured glucose concentration data, including glucose concentration values, to a display device via wireless connection.

For example, the at least one sensor electronics module may be configured to sample the analog electrical signals at a particular sampling period (or rate), such as every 1 second (1 Hz), 5 seconds, 10 seconds, 30 seconds, 1 minute, 3 minutes, 5 minutes, etc., and to transmit the measured glucose concentration data to a display device at a particular transmission period (or rate), which may be the same as (or longer than) the sampling period, such as every 1 minute (0.016 Hz), 5 minutes, 10 minutes, 15 minutes etc.

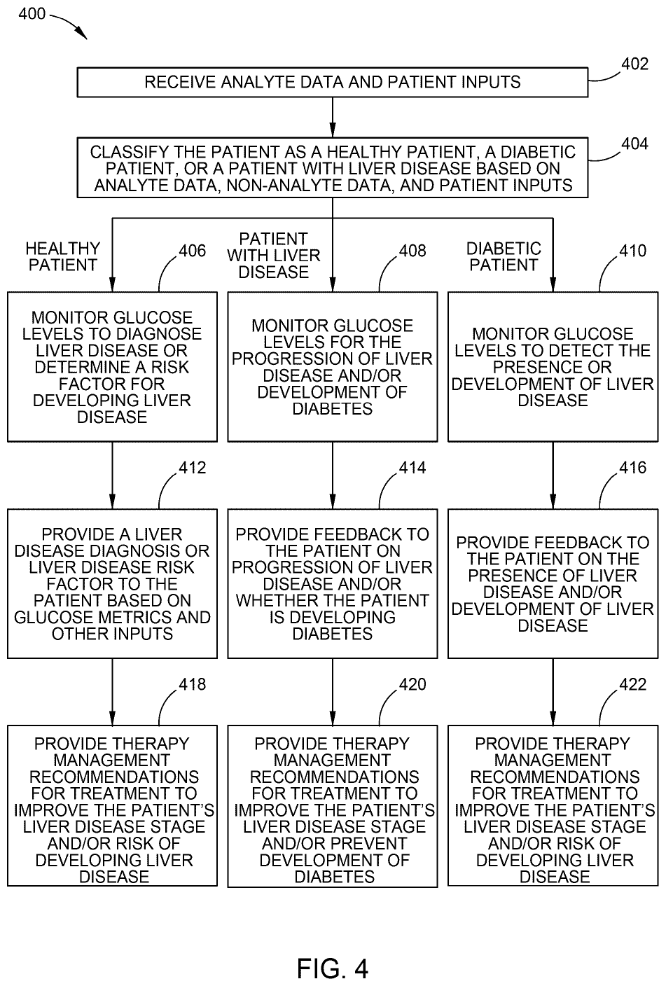

The real-time analyte data that is continuously generated by the continuous analyte monitoring system described herein, and therefore, allows the therapy management system herein to perform any or all of the functions to: determine a risk or presence of liver disease or diabetes, determine the progression of liver disease or diabetes, as well as provide therapy management recommendations, in real-time, which is technically impossible to perform using existing or conventional techniques or systems. Further, because of the real-time nature of this data, it is also humanly impossible to continuously process a real-time data stream of analyte values over time to derive meaningful data and insight using the algorithms and systems described herein for determining a risk or presence of liver disease or diabetes, determining the progression of liver disease or diabetes, as well as providing therapy management recommendations. In other words, deriving meaningful data and insight from a stream of real-time data that is continuously generated, processed, calibrated, and analyzed, using the algorithms and systems described herein, is not a task that can be mentally performed. For example, executing the algorithm described in relation to FIG. 4 in real-time and on a continuous basis, which would involve using a stream of real-time data that is continuously generated by a continuous analyte monitoring system worn by a host and/or significantly large amount of population data (e.g., hundreds or thousands of data points for each one of thousands or millions of users in the user population) is not a task that can be mentally performed, especially in real-time at times.

Further, the real-time data generated is not merely data, but data that is processed in a particular manner to allow for use in algorithms that require a certain type of data. Each analyte sensor system that is manufactured by a sensor manufacturer might perform slightly different. As such, there might be inconsistencies between sensors and the measurements they generate once in use. Accordingly, certain embodiments herein are directed to determining the performance of an analyte sensor system during a manufacturing calibration process (in vitro), which includes quantifying certain sensor operating parameters, such as a calibration slope (also known as calibration sensitivity), a calibration baseline, etc.

Generally, calibration sensitivity refers to the amount of electrical current produced by an analyte sensor of an analyte sensor system when immersed in a predetermined amount of a measured analyte. The amount of electrical current may be expressed in units of picoAmps (pA) or counts. The amount of measured analyte may be expressed as a concentration level in units of milligrams per deciliter (mg/dL), and the calibration sensitivity may be expressed in units of pA/(mg/dL) or counts/(mg/dL). The calibration baseline refers to the amount of electrical current produced by the analyte sensor when no analyte is detected, and may be expressed in units of pA or counts.

The calibration sensitivity, calibration baseline, and other information related to the sensitivity profile for the analyte sensor system may be programmed into the sensor electronics module of the analyte sensor system during the manufacturing process, and then used to convert the analyte sensor electrical signals into measured analyte concentration levels. For example, the calibration slope (calibration sensitivity) may be used to predict an initial in vivo sensitivity (M₀) and a final in vivo sensitivity (M_{f}), which are programmed into the sensor electronics module and used to convert the analyte sensor electrical signals into measured analyte concentration levels.

In certain embodiments, during in vivo use, the sensor electronics module of an analyte sensor system samples the analog electrical signals produced by the analyte sensor to generate analyte sensor count values, and then determines the measured analyte concentration levels based on the analyte sensor count values, the initial in vivo sensitivity (M₀), and the final in vivo sensitivity (M_{f}). For example, measured analyte concentration levels may be determined using a sensitivity function M(t) that is based on the initial in vivo sensitivity (M₀) and the final in vivo sensitivity (M_{f}). The sensitivity function M(t) may expressed in several different ways, such as a simple correction factor that is not dependent on elapsed time (tᵢ) of in vivo use, a linear relationship between sensitivity and time (tᵢ), an exponential relationship between sensitivity and time (tᵢ), etc. Equation 1 presents one technique for determining a measured analyte concentration level (ACL) from an analyte sensor count value (count) at a time tᵢ:

$$A C L = count / M ( {t}_{i} ) \qquad Eq . 1$$

A calibration baseline (baseline) may also be used to determine a measured analyte concentration level (ACL) from an analyte sensor count value (count) at a time tᵢ, and Equation 2 presents one technique:

$$ACL = ( count - baseline ) / M ( ti ) \qquad Eq . 2$$

In addition, to allow for processing of the data in a distributed manner, various communication mechanisms must be used to transmit the data in processed or unprocessed format across multiple devices having various processing capabilities. Due to the sensitivity of health related data, as well as the danger to a user from any external malicious attempts, such transmissions must be performed in a secure and efficient manner. Example techniques for such data transmission and communication are described herein. But other transmission techniques may similarly be used.

### Example Therapy Management System Including an Example Analyte Sensor for Predicting Current or Future Diabetes or Liver Disease State

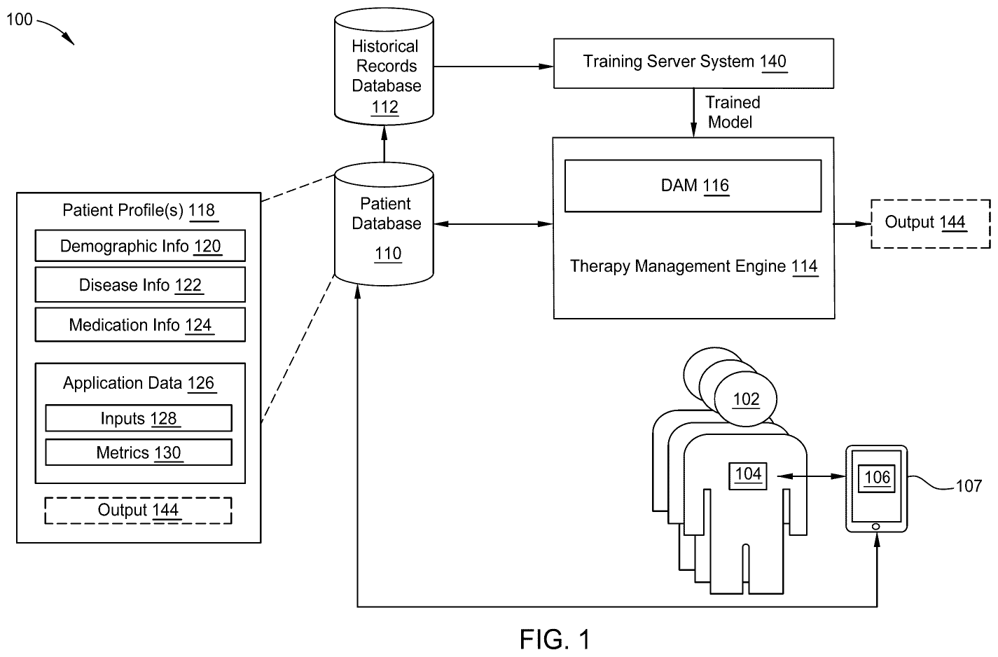

FIG. 1 illustrates an example therapy management system 100 for predicting a current or future disease state of patients 102 (individually referred to herein as a patient and collectively referred to herein as patients), using a continuous analyte monitoring system 104 configured to continuously measure analyte levels including one or both glucose and lactate levels, as well as other analytes if necessary. As described herein, a current or future disease state may include a level of risk of liver disease or diabetes, a progression or regression of liver disease or diabetes, a presence of dysglycemia or organ dysfunctions that may be linked to liver disease or diabetes. A patient, in certain embodiments, is a patient with liver disease, a healthy patient (e.g., a patient not diagnosed with liver disease and/or diabetes), or a diabetic patient.

In certain embodiments, therapy management system 100 includes continuous analyte monitoring system 104, a display device 107 that executes application 106, a therapy management engine 114, a patient database 110, a historical records database 112, a training server system 140, and a therapy management engine 114, each of which is described in more detail below.

The term “analyte” as used herein is a broad term used in its ordinary sense, including, without limitation, to refer to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. Analytes for measurement by the devices and methods may include, but may not be limited to, potassium, glucose, endogenous insulin, acarboxyprothrombin; acylcarnitine; endogenous insulin; adenine phosphoribosyl transferase; adenosine deaminase; albumin; albumin-creatinine ratio; alpha-fetoprotein; amino acid profiles (arginine (Krebs cycle), histidine/urocanic acid, homocysteine, phenylalanine/tyrosine, tryptophan); androstenedione; antipyrine; arabinitol enantiomers; arginase; benzoylecgonine (cocaine); biotinidase; biopterin; c-peptide; c-reactive protein; carnitine; carnosinase; CD4; ceruloplasmin; chenodeoxycholic acid; chloroquine; cholesterol; cholinesterase; conjugated 1-β hydroxy-cholic acid; cortisol; cortisone; creatine kinase; creatine kinase MM isoenzyme; creatinine; cyclosporin A; cystatin C; d-penicillamine; de-ethylchloroquine; dehydroepiandrosterone sulfate; DNA (acetylator polymorphism, alcohol dehydrogenase, alpha 1-antitrypsin, glucose-6-phosphate dehydrogenase, hemoglobin A, hemoglobin S, hemoglobin C, hemoglobin D, hemoglobin E, hemoglobin F, D-Punjab, hepatitis B virus, HCMV, HIV-1, HTLV-1, MCAD, RNA, PKU, Plasmodium vivax, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; pyruvate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; proteinuria; quinine; reverse tri-iodothyronine (rT3); selenium; serum pancreatic lipase; sisomicin; somatomedin C; specific antibodies recognizing any one or more of the following that may include (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, pro-C3, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin.

Salts, sugar, protein, fat, vitamins, and hormones (e.g., insulin) naturally occurring in blood or interstitial fluids can also constitute analytes in certain implementations. The analyte can be naturally present in the biological fluid, for example, a metabolic product, a hormone, an antigen, an antibody, and the like. Alternatively, the analyte can be introduced into the body or exogenous, for example, a contrast agent for imaging, a radioisotope, a chemical agent, a fluorocarbon-based synthetic blood, or a drug or pharmaceutical composition, including but not limited to insulin; glucagon, ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), and intermediaries in the Citric Acid Cycle.

While the analytes that are measured and analyzed by the devices and methods described herein include glucose, in some cases other analytes listed above may also be considered.

In certain embodiments, continuous analyte monitoring system 104 is configured to continuously measure one or more analytes and transmit the analyte measurements to an electric medical records (EMR) system (not shown in FIG. 1). An EMR system is a software platform which allows for the electronic entry, storage, and maintenance of digital medical data. An EMR system is generally used throughout hospitals and/or other caregiver facilities to document clinical information on patients over long periods. EMR systems organize and present data in ways that assist clinicians with, for example, interpreting health conditions and providing ongoing care, scheduling, billing, and follow up. Data contained in an EMR system may also be used to create reports for clinical care and/or disease management for a patient. In certain embodiments, the EMR is in communication with therapy management engine 114 (e.g., via a network) for performing the techniques described herein. In particular, as described herein, therapy management engine 114 may obtain data associated with a patient, use the obtained data as input into one or more trained model(s), and output a prediction. In some cases, the EMR may provide the data to therapy management engine 114 to be used as input into the one or more models. Further, in some cases, therapy management engine 114, after making a prediction, may provide the output prediction to the EMR.

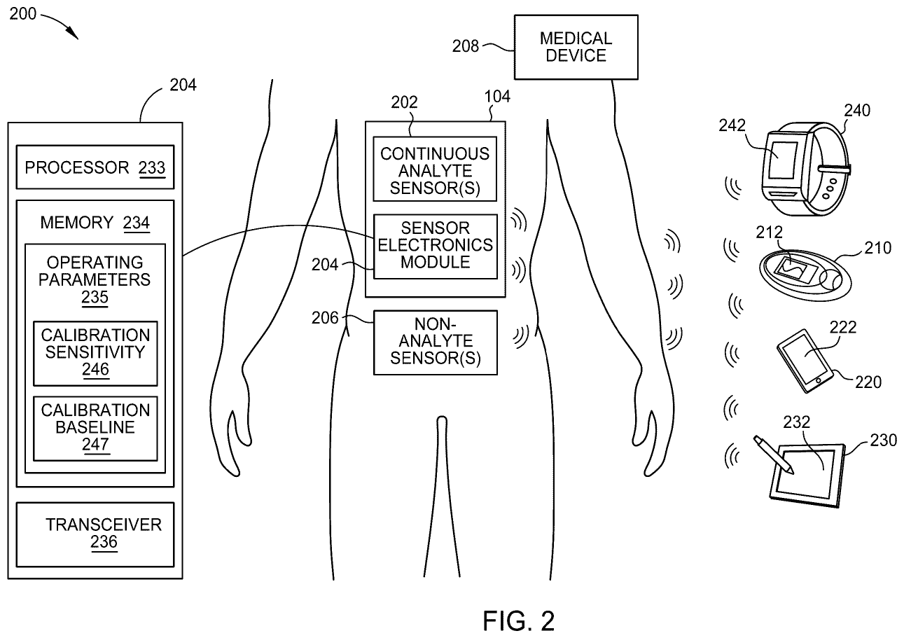

In certain embodiments, continuous analyte monitoring system 104 is configured to continuously measure one or more analytes and transmit the analyte measurements to display device 107 for use by application 106. In some embodiments, continuous analyte monitoring system 104 transmits the analyte measurements to display device 107 through a wireless connection (e.g., Bluetooth connection). In certain embodiments, display device 107 is a smart phone. However, in certain other embodiments, display device 107 may instead be any other type of computing device such as a laptop computer, a smart watch, a tablet, or any other computing device capable of executing application 106. In some embodiments, continuous analyte monitoring system 104 and/or analyte sensor application 106 transmit the analyte measurements to one or more other individuals having an interest in the health of the patient (e.g., a family member or physician for real-time treatment and care of the patient). Continuous analyte monitoring system 104 may be described in more detail with respect to FIG. 2.

Application 106 is a mobile health application (or similar software program) that is configured to receive and analyze analyte measurements from continuous analyte monitoring system 104. In particular, application 106 stores information about a patient, including the patient's analyte measurements, in a patient profile 118 associated with the patient for processing and analysis, as well as for use by therapy management engine 114 to provide therapy management recommendations or guidance to the patient.

Therapy management engine 114 refers to a set of software instructions with one or more software modules, including data analysis module (DAM) 116. In certain embodiments, therapy management engine 114 executes entirely on one or more computing devices in a private or a public cloud. In such embodiments, application 106 communicates with therapy management engine 114 over a network (e.g., Internet). In some other embodiments, therapy management engine 114 executes partially on one or more local devices, such as display device 107 and/or continuous analyte monitoring system 104, and partially on one or more computing devices in a private or a public cloud. In some other embodiments, therapy management engine 114 executes entirely on one or more local devices, such as display device 107 and/or continuous analyte monitoring system 104.

As discussed in more detail herein, therapy management engine 114 may provide therapy management recommendations to the patient via application 106 for treatment recommendations to improve the patient's disease management (e.g., liver or diabetes or both), decrease the risk of developing the disease, and/or prevent the patient from developing a disease. In certain embodiments, treatment recommendations include Therapy management engine 114 provides therapy management recommendations for treatment based on information included in patient profile 118.

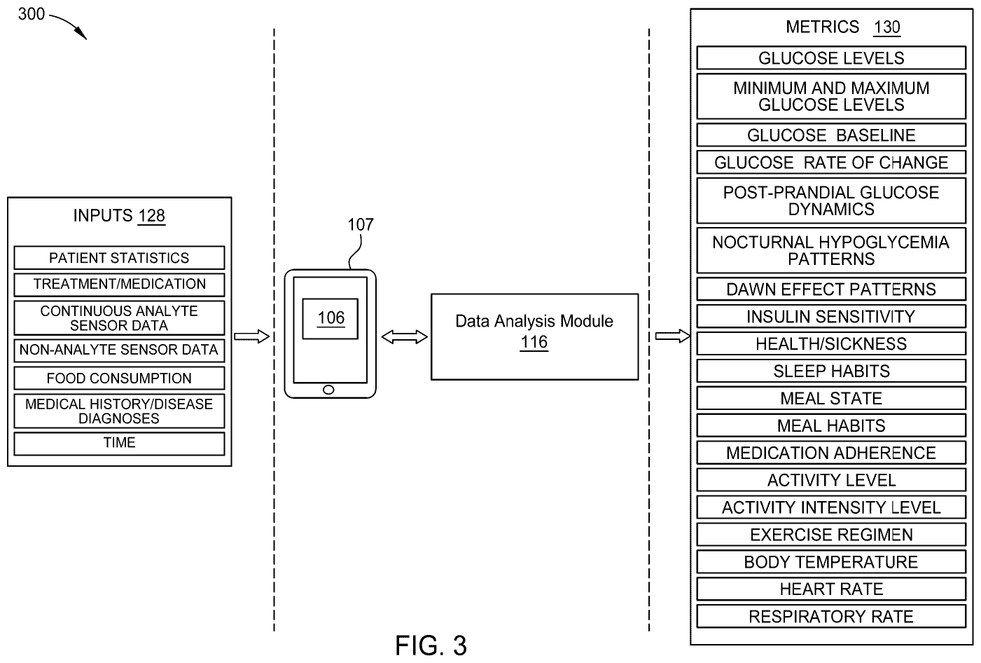

Patient profile 118 may include information collected about the patient from application 106. For example, application 106 provides a set of inputs 128, including the analyte measurements received from continuous analyte monitoring system 104, that are stored in patient profile 118. In certain embodiments, inputs 128 provided by application 106 include other data in addition to analyte measurements received from continuous analyte monitoring system 104. For example, application 106 may obtain additional inputs 128 through manual patient input, one or more other non-analyte sensors or devices, non-continuous analyte lab test results (e.g., liver biopsy, metabolic assay panels, Fibroscan results, etc.), other applications executing on display device 107, etc. Non-analyte sensors and devices include one or more of, but are not limited to, an insulin pump, an electrocardiogram (ECG) sensor, a heart rate monitor, a blood pressure sensor, a respiratory sensor, a thermometer, sensors or devices provided by display device 107 (e.g., accelerometer, camera, global positioning system (GPS), heart rate monitor, etc.) or other patient accessories (e.g., a smart watch, or a continuous positive airway (CPAP) machine), or any other sensors or devices that provide relevant information about the patient. Inputs 128 of patient profile 118 provided by application 106 are described in further detail below with respect to FIG. 3.

DAM 116 of therapy management engine 114 is configured to process the set of inputs 128 to determine one or more metrics 130. Metrics 130, discussed in more detail below with respect to FIG. 3, may, at least in some cases, be generally indicative of the disease state of a patient, such as one or more of the patient's general analyte trends, trends associated with the health of the patient, trends associated with activities of the patient such as meal times and/or exercise sessions of the patient, etc. In certain embodiments, metrics 130 are then used by therapy management engine 114 as input for determining a risk or presence of liver disease or diabetes, and/or determining a progression of liver disease or diabetes to provide to a patient. As shown, metrics 130 are also stored in patient profile 118.

Patient profile 118 also includes demographic info 120, disease progression info 122, and/or medication info 124. In certain embodiments, such information is provided through patient input or obtained from certain data stores (e.g., electronic medical records (EMRs), etc.). In certain embodiments, demographic info 120 includes one or more of the patient's age, body mass index (BMI), ethnicity, gender, etc. In certain embodiments, disease progression info 122 includes information about a disease of a patient, such as whether the patient has been previously diagnosed with liver disease, diabetes, and/or have had symptoms of such diseases, such as a history of hyperglycemia, hypoglycemia, etc. In certain embodiments, information about a patient's disease also includes the length of time since diagnosis, the level of disease control, level of compliance with disease management therapy, predicted liver function, other types of diagnosis (e.g., heart disease, hypertension, obesity), or measures of health (e.g., heart rate, exercise, sleep, etc.), and/or the like. In certain embodiments, patient profile 118 includes information about the patient's drug and/or alcohol consumption, which may affect a patient's lactate metabolism and/or liver function.

In certain embodiments, medication info 124 includes information about the amount, frequency, and type of a medication taken by a patient. In certain embodiments, the amount, frequency, and type of a medication taken by a patient is time-stamped and correlated with the patient's analyte levels, thereby, indicating the impact the amount, frequency, and type of the medication had on the patient's analyte levels.

In certain embodiments, medication information includes information about the consumption of one or more drugs known to cause disease progression. These medications include those that damage the liver (e.g., affect lactic clearance) and/or lead to liver toxicity. One or more drugs known to damage the liver and/or lead to liver toxicity may include antibiotics such as amoxicillin/clavulanate, clindamycin, erythromycin, nitrofurantoin, rifampin, sulfonamides, tetracyclines, trimethoprim/sulfamethoxazole, and drugs used to treat tuberculosis (isoniazid and pyrazinamide), anticonvulsants such as tarbamazepine, thenobarbital, phenytoin, and valproate, antidepressants such as bupropion, fluoxetine, mirtazapine, paroxetine, sertraline, trazodone, and tricyclic antidepressants such as amitriptyline, antifungal drugs such as ketoconazole and terbinafine, antihypertensive drugs (e.g., drugs used to treat high blood pressure or sometimes kidney or heart disorder) such as captopril, enalapril, irbesartan, lisinopril, losartan, and verapamil, antipsychotic drugs such as phenothiazines (e.g., such as chlorpromazine) and risperidone, heart drugs such as amiodarone and clopidogrel, hormone regulation drugs such as anabolic steroids, birth control pills (oral contraceptives), and estrogens, pain relievers such as acetaminophen and nonsteroidal anti-inflammatory drugs (NSAIDs), and other drugs such as acarbose (e.g., used to treat diabetes), allopurinol (e.g., used to treat gout), antiretroviral therapy (ART) drugs (e.g., used to treat human immunodeficiency virus (HIV) infection), baclofen (e.g., a muscle relaxant), cyproheptadine (e.g., an antihistamine), azathioprine (e.g., used to prevent rejection of an organ transplant), methotrexate (e.g., used to treat cancer), omeprazole (e.g., used to treat gastroesophageal reflux), PD-1/PD-L1 inhibitors (e.g., anticancer drugs), statins (e.g., used to treat high cholesterol levels), and many types of chemotherapy, including immune checkpoint inhibitors.

In certain embodiments, medication information includes information about consumption of one or more drugs known to improve the body's glycemic or metabolic conditions (e.g., improved in organ function). Such drugs include ademetionine, avatrombopag, dehydroemetine, entecavir, glecaprevir and pibrentasvir, glucagon-like peptide 1 agonists (GLP-1), lamivudine, metadoxine, methionine, sodium-glucose cotransporter-2 (SGLT2), sofosbuvir, velpatasvir, and voxilaprevir, telbivudine, tenofovir, trientine, ursodeoxycholic acid, and the like.

The information regarding medication consumption may be input by a user, determined using electronic patient records, or may be detected using technologies that allow for detection of presence of certain chemicals in the body.

In certain embodiments, patient profile 118 is dynamic because at least part of the information that is stored in patient profile 118 may be revised over time and/or new information may be added to patient profile 118 by therapy management engine 114 and/or application 106. Accordingly, information in patient profile 118 stored in patient database 110 provides an up-to-date repository of information related to a patient.

Patient database 110, in some embodiments, refers to a storage server that operates in a public or private cloud. Patient database 110 may be implemented as any type of datastore, such as relational databases, non-relational databases, key-value datastores, file systems including hierarchical file systems, and the like. In some exemplary implementations, patient database 110 is distributed. For example, patient database 110 may comprise a plurality of persistent storage devices, which are distributed. Furthermore, patient database 110 may be replicated so that the storage devices are geographically dispersed.

Patient database 110 includes patient profiles 118 associated with a plurality of patients who similarly interact with application 106 executing on the display devices 107 of the other patients. Patient profiles stored in patient database 110 are accessible to not only application 106, but therapy management engine 114, as well. Patient profiles in patient database 110 may be accessible to application 106 and therapy management engine 114 over one or more networks (not shown). As described above, therapy management engine 114, and more specifically DAM 116 of therapy management engine 114, can fetch inputs 128 from patient database 110 and compute a plurality of metrics 130 which can then be stored as application data 126 in patient profile 118.

In certain embodiments, patient profiles 118 stored in patient database 110 are also stored in historical records database 112. Patient profiles 118 stored in historical records database 112 may provide a repository of up-to-date information and historical information for each patient of application 106. Thus, historical records database 112 essentially provides all data related to each patient of application 106, where data is stored according to an associated timestamp. The timestamp associated with information stored in historical records database 112 may identify, for example, when information related to a patient has been obtained and/or updated.

Further, historical records database 112 may maintain time series data collected for patients over a period of time, including for patients who use continuous analyte monitoring system 104 and application 106. For example, analyte data for a patient who has used continuous analyte monitoring system 104 and application 106 for a period of five years to manage the patient's liver condition and/or diabetes may have time series analyte data associated with the patient maintained over the five-year period.

Further, in certain embodiments, historical records database 112 includes data for one or more patients who are not patients of continuous analyte monitoring system 104 and/or application 106. For example, historical records database 112 may include information (e.g., patient profile(s)) related to one or more patients analyzed by, for example, a healthcare physician (or other known method), and not previously diagnosed with liver disease and/or diabetes, as well as information (e.g., patient profile(s)) related to one or more patients who were analyzed by, for example, a healthcare physician (or other known method) and were previously diagnosed with (varying types and stages of) liver disease and/or diabetes. Data stored in historical records database 112 may be referred to herein as population data, which could include hundreds or thousands of data points for each one of thousands or millions of users in the user population. In other words, data stored in historical records database 112 and used in certain embodiments described herein could include gigabytes, terabytes, petabytes, exabytes, etc. of data.

Data related to each patient stored in historical records database 112 may provide time series data collected over the disease lifetime of the patient. For example, the data may include information about the patient prior to being diagnosed with liver disease and/or diabetes and information associated with the patient during the lifetime of the disease, including information related to each stage of the liver disease and/or diabetes as it progressed and/or regressed in the patient, as well as information related to other diseases, such as hyperglycemia, hypoglycemia, kidney conditions and diseases, hypertension, or similar diseases that are co-morbid in relation to liver disease and/or diabetes. Such information may indicate symptoms of the patient, physiological states of the patient, glucose levels of the patient, states/conditions of one or more organs of the patient, habits of the patient (e.g., activity levels, food consumption, etc.), medication prescribed, etc. throughout the lifetime of the disease.

Although depicted as separate databases for conceptual clarity, in some embodiments, patient database 110 and historical records database 112 may operate as a single database. That is, historical and current data related to patients of continuous analyte monitoring system 104 and application 106, as well as historical data related to patients that were not previously patients of continuous analyte monitoring system 104 and application 106, may be stored in a single database. The single database may be a storage server that operates in a public or private cloud.

As mentioned previously, therapy management system 100 is configured to predict the liver health of the patient, including diagnosing, staging, and assessing risks of liver disease, as well as predict the risk, presence, or severity of diabetes in a patient using continuous analyte monitoring system 104, including, at least, a continuous glucose monitor (CGM). In certain embodiments, therapy management engine 114 is configured to provide real-time and or non-real-time therapy management around liver health and diabetes to the patient and or others, including but not limited, to healthcare providers, family members of the patient, caregivers of the patient, researchers, artificial intelligence (AI) engines, and/or other individuals, systems, and/or groups supporting care or learning from the data.

For example, therapy management engine 114 may be used to collect information associated with a patient in patient profile 118 stored in patient database 110, to perform analytics thereon for predicting liver disease and diabetes diagnoses, progression, and risk and, in some cases, providing therapy management recommendations to the patient based on the prediction. Therapy management engine 114 may also be used to collect information associated with a patient in patient profile 118 to perform analytics thereon for determining the presence and/or severity of liver disease and/or diabetes for the patient and providing therapy management recommendations for treatment recommendations based, at least in part, on the determination. Patient profile 118 may be accessible to therapy management engine 114 over one or more networks (not shown) for performing such analytics.

In certain embodiments, therapy management engine 114 further collects information from the patient regarding recent habits in order to determine the accuracy of a risk assessment or diagnosis. For example, therapy management engine 114 may determine, from continuous analyte monitoring system 104, an abnormal pattern of analyte data consistent with worsening disease state (which may be due to various factors). In some examples, therapy management engine 114 may determine, from continuous analyte monitoring system 104, an abnormal pattern of analyte data consistent with reduced liver function. During time periods of abnormal analyte patterns, therapy management engine 114 may review information collected from the patient to determine whether the abnormal analyte pattern is a result of worsening disease state, worsening liver function, or other factors that may lead to change in disease state. Therapy management engine 114 can further determine if the abnormal pattern is a result of other factors such as recent alcohol consumption, a new medication, or an illness or infection. Based on information from the patient, therapy management engine 114 may accurately determine whether abnormal analyte data is a result of worsening liver function, disease state, or a result of other factors such as temporary illness, alcohol consumption, or new medications.

In certain embodiments, therapy management system 100 is designed to predict the risk or likelihood of one or more symptoms of liver disease and/or diabetes occurring in real-time (including near real-time), or within a specified period of time for a patient. In certain embodiments, to enable such prediction, therapy management engine 114 is configured to collect information associated with a patient in patient profile 118 stored in patient database 110, to perform analytics thereon to classify whether the patient is (1) a healthy patient (e.g., is not diagnosed with, or showing signs of, diabetes or liver disease), (2) a patient with diabetes, or (3) a patient with liver disease. Based on this classification, therapy management engine 114 may (1) monitor the patient for the development of liver disease and/or assess liver disease risk, (2) determine whether a patient with diabetes also has liver disease, and/or (3) monitor for liver disease progression and/or development of diabetes, respectively. In certain embodiments, therapy management engine 114 monitors the patient for development of liver disease, monitor the patient for progression of liver disease, assess liver disease risk, and/or diagnose liver disease without a patient classification. In certain embodiments, therapy management engine 114 monitors the patient for development of diabetes, monitor the patient for progression of diabetes, assess diabetes risk, and/or diagnose diabetes without a patient classification. Patient profile 118 may be accessible to therapy management engine 114 over one or more networks (not shown) for performing such analytics.

In certain embodiments, therapy management engine 114 utilizes one or more trained machine learning models capable of making predictions based on information that therapy management engine 114 has collected/received from patient profile 118. In the illustrated embodiment of FIG. 1, therapy management engine 114 may utilize trained machine learning model(s) provided by a training server system 140. Although depicted as a separate server for conceptual clarity, in certain embodiments, training server system 140 and therapy management engine 114 operates as a single server or system. That is, the model may be trained and used by a single server, or may be trained by one or more servers and deployed for use on one or more other servers or systems. In certain embodiments, the model is trained on one or many virtual machines (VMs) running, at least partially, on one or many physical services in relational and or non-relational database formats. In some examples, the machine learning model may be trained and or run on a local device such as the patient's device or a device of a caregiver, health care professional, or any other local device.

Training server system 140 is configured to train the machine learning model(s) using training data, which may include data (e.g., from patient profiles) associated one or more patients (e.g., patients or non-patients of continuous analyte monitoring system 104 and/or application 106) previously diagnosed with varying stages of liver disease, previously diagnosed with varying stages of diabetes, as well as patients not previously diagnosed with liver disease or diabetes (e.g., healthy patients, etc.). The training data may be stored in historical records database 112 and may be accessible to training server system 140 over one or more networks (not shown) for training the machine learning model(s).

The training data refers to a dataset that has been featurized and labeled. For example, the dataset may include a plurality of data records, each including information corresponding to a different patient profile stored in patient database 110, where each data record is featurized and labeled. In machine learning and pattern recognition, a feature is an individual measurable property or characteristic. Generally, the features that best characterize the patterns in the data are selected to create predictive machine learning models. Data labeling is the process of adding one or more meaningful and informative labels to provide context to the data for learning by the machine learning model.

As an illustrative example, each relevant characteristic of a patient, which is reflected in a corresponding data record, may be a feature used in training the machine learning model. Such features may include demographic information (e.g., age, gender, ethnicity, etc.), analyte information (e.g., glucose metrics, such as a glucose baseline, minimum and maximum daily glucose levels, glucose peak following meals, drinks, or food, glucose clearance rate following glucose peak, and/or glucose levels during and after exercise, etc.), non-analyte sensor information (e.g., heart rate, temperature, etc.), liver disease information (e.g., liver disease diagnosis and staging), diabetes information (e.g., diabetes diagnosis, insulin resistance), comorbidities (e.g., hyperglycemia, hypoglycemia, kidney conditions and diseases, hypertension, etc.), medication information, and/or any other information relevant to classifying patients and/or providing disease diagnosis and stage predictions, or recommendations for treatment to patients.

In addition, the data record is labeled with information the corresponding model is being trained to predict. In one example, if a model is being trained to classify a patient into a healthy patient, a patient with liver disease, or a diabetic patient, then the data records in the training dataset are labeled with such classification. In another example, if a model is being trained to output a liver disease diagnosis and/or diabetes diagnosis prediction, then the data records in the training dataset are labeled with one or more of such predictions. Note that, in one example, such a model may be a multi-input single-output (MISO) model, configured to predict only one disease diagnosis (e.g., liver disease), in which case additional MISO models may be trained for each predicting one of other disease-related predictions (e.g., liver disease stage, diabetes diagnosis prediction, etc.). In another example, such a model may be a multi-input multi-output (MIMO) model, configured to predict multiple disease-related predictions (e.g., liver disease diagnosis, liver disease stage, diabetes diagnosis prediction, etc.).

The model(s) are then trained by training server system 140 using the featurized and labeled training data. In particular, the features of each data record may be used as input into the machine learning model(s), and the generated output may be compared to label(s) associated with the corresponding data record. The model(s) may compute a loss based on the difference between the generated output and the provided label(s). This loss is then used to modify the internal parameters or weights of the model. By iteratively processing each data record corresponding to each historical patient, the model(s) may be iteratively refined to generate accurate predictions of a patient's classification, disease state, recommendations for treatment, etc.

As illustrated in FIG. 1, training server system 140 deploys these trained model(s) to therapy management engine 114 for use during runtime. For example, therapy management engine 114 may obtain patient profile 118 associated with a patient and stored in patient database 110, use information in patient profile 118 as input into the trained model(s), and output a prediction indicative of the patient's classification as a healthy patient, a patient with liver disease, or a diabetic patient, and/or feedback related to diabetes and/or liver disease state (e.g., shown as output 144 in FIG. 1). Output 144 generated by therapy management engine 114 may also indicate improvement in the patient's liver disease and/or diabetes over time. Output 144 may be provided to the patient (e.g., through application 106), to a caretaker of the patient (e.g., a parent, a relative, a guardian, a teacher, a physical therapist, a fitness trainer, a nurse, etc.), to a physician or healthcare provider of the patient, or any other individual that has an interest in the wellbeing of the patient for purposes of improving the health of the patient, such as, in some cases by effectuating recommended treatment. Output 144 generated by therapy management engine 114 is stored in patient database 110 and is utilized to train or re-train the trained model(s).

In certain embodiments, output 144 generated by therapy management engine 114 is stored in patient profile 118. Output 144 may be indicative of a patient classification (e.g., healthy patient, patient with liver disease, or a diabetic patient), current or future disease state, recommendations for treatment, etc. Output 144 stored in patient profile 118 may be continuously updated by therapy management engine 114. Accordingly, for example, disease states and recommendations, originally stored as outputs 144 in patient profile 118 in patient database 110 and then passed to historical records database 112, may provide an indication of the progression or improvement of the disease state of a patient over time, as well as provide an indication as to the effectiveness of different treatments, medications, and lifestyle changes recommended to the patient to improve disease state.

In certain embodiments, a patient's own historical data is used by training server system 140 to train a personalized model for the patient that provides therapy management and insight around the patient's medical history/current disease state, average glucose levels, etc. For example, in certain embodiments, a model trained based on population data is used to provide disease state feedback to the patient. However, after collecting personalized information (e.g., analyte sensor information, non-analyte sensor information, disease state, etc.) associated with the patient, the personalized information may be used for further individualization or personalization of the model. For example, information obtained over time from the patient may be used to more accurately determine disease state, provide personalized recommendations for treatment, and monitor progression and/or regression of disease state over time.

Further, a patient's historical data can be used as a baseline to indicate progression or regression in the patient's disease state based on various criteria. This criteria is selected from a list of available variables including the patient's glucose baseline, the patient's glucose response following a meal or food, or other information that indicate a glycemic response or liver function indication. As an illustrative example, a patient's data from 2 weeks ago may be used as a baseline that can be compared with the patient's current data to identify whether the patient's disease state has changed (e.g., an improvement or worsening). In an additional or alternative embodiment, the model is further able to predict or project out the patient's disease state or its future improvement/deterioration based on the patient's recent pattern of data (e.g., analyte data, meal trends, exercise trends, etc.).

In certain embodiments, an AI/ML model is trained to provide treatment recommendations, and other types of therapy management recommendations to help the patient improve their disease state based on the patient's historical data, including how different types of activity impacted the patient's glucose levels historically. In certain embodiments, an AI/ML model is trained to predict the underlying cause of certain improvements or deteriorations in the patient's disease state. For example, application 106 may display a patient interface with a graph that shows the patient's glucose levels (e.g., disease state) with trend lines and indicate, e.g., retrospectively, how the body's ability to clear glucose suffered at certain points in time. Other depictions or trend indications, variables or metrics may also be provided for display to indicate the change.

In certain other embodiments, rules-based models may be used. For example, a rules-based model may be used to map a patient's inputs and/or historical data to certain patient classifications, current or future disease state, recommendations for treatment, etc., using a rules library. In certain embodiments, a rules-based model maps certain inputs to patient classifications, disease state predictions, and/or recommendations for patients with similar inputs in the past. Some example rules are discussed herein in relation to blocks 404 and 412.

FIG. 2 is a diagram 200 conceptually illustrating an example continuous analyte monitoring system 104 including example continuous analyte sensor(s) with sensor electronics, in accordance with certain aspects of the present disclosure. For example, continuous analyte monitoring system 104 may be configured to continuously monitor one or more analytes of a patient, in accordance with certain aspects of the present disclosure.

Continuous analyte monitoring system 104 in the illustrated embodiment includes sensor electronics module 204 and one or more continuous analyte sensor(s) 202 (individually referred to herein as continuous analyte sensor 202 and collectively referred to herein as continuous analyte sensors 202) associated with sensor electronics module 204. Sensor electronics module 204 may be in wireless communication (e.g., directly or indirectly) with one or more of display devices 210, 220, 230, and 240. In certain embodiments, sensor electronics module 204 is also in wireless communication (e.g., directly or indirectly) with one or more medical devices, such as medical devices 208 (individually referred to herein as medical device 208 and collectively referred to herein as medical devices 208), and/or one or more other non-analyte sensors 206 (individually referred to herein as non-analyte sensor 206 and collectively referred to herein as non-analyte sensor 206).

In certain embodiments, a continuous analyte sensor 202 comprises one or more sensors for detecting and/or measuring analyte(s). The continuous analyte sensor 202 may be a multi-analyte sensor configured to continuously measure two or more analytes or one or more single analyte sensors each configured to continuously measure a single analyte as a non-invasive device, a subcutaneous device, a transcutaneous device, a transdermal device, and/or an intravascular device. In certain embodiments, multiple single continuous analyte sensors in communication with the same sensor electronics module 204 and/or the same display device can be implemented in lieu of a single analyte sensor.

In certain embodiments, the continuous analyte sensor 202 is configured to continuously measure analyte levels of a patient using one or more techniques, such as enzymatic techniques, chemical techniques, physical techniques, electrochemical techniques, spectrophotometric techniques, polarimetric techniques, calorimetric techniques, iontophoretic techniques, radiometric techniques, immunochemical techniques, and the like. The term “continuous,” as used herein, can mean fully continuous, semi-continuous, periodic, etc. In certain aspects, the continuous analyte sensor 202 provides a data stream indicative of the concentration of one or more analytes in the patient. The data stream may include raw data signals, which are then converted into a calibrated and/or filtered data stream used to provide estimated analyte value(s) to the patient.

In certain embodiments, one or more multi-analyte sensors are used in combination with one or more single analyte sensors. Information from each of the multi-analyte sensor(s) and single analyte sensor(s) may be combined to provide therapy management using methods described herein. In further embodiments, other non-contact and or periodic or semi-continuous, but temporally limited, measurements for physiological information are integrated into the system such as by including weight scale information or non-contact heart rate monitoring from a sensor pad under the patient while in a chair or bed, through an infra-red camera detecting temperature and/or blood flow patterns of the patient, and/or through a visual camera with machine vision for height, weight, or other parameter estimation without physical contact.

In certain embodiments, the continuous analyte sensor(s) 202 comprises a percutaneous wire that has a proximal portion coupled to the sensor electronics module 204 and a distal portion with several electrodes, such as a measurement electrode and a reference electrode. The measurement (or working) electrode may be coated, covered, treated, embedded, etc., with one or more chemical molecules that react with a particular analyte, and the reference electrode may provide a reference electrical voltage. The measurement electrode may generate the analog electrical signal, which is conveyed along a conductor that extends from the measurement electrode to the proximal portion of the percutaneous wire that is coupled to the sensor electronics module 204. After the continuous analyte monitoring system 104 has been applied to epidermis of the patient, continuous analyte sensor(s) 202 penetrates the epidermis, and the distal portion extends into the dermis and/or subcutaneous tissue under epidermis. Other configurations of continuous analyte sensor(s) 202 may also be used, such as a multi-analyte sensor that includes multiple measurement electrodes, each generating an analog electrical signal that represents the concentration levels of a particular analyte.

Generally, a single-analyte sensor generates an analog electrical signal that is proportional to the concentration level of a particular analyte. Similarly, each multi-analyte sensor generates multiple analog electrical signals, and each analog electrical signal is proportional to the concentration level of a particular analyte. As an illustrative example, continuous analyte sensor 202 may include a single-analyte sensor configured to measure glucose concentration levels, and another single-analyte sensor configured to measure another analyte concentration level of the patient. As another illustrative example, continuous analyte sensor(s) 202 may include a single-analyte sensor configured to measure glucose concentration levels, and one or more multi-analyte sensors configured to measure lactate concentration levels, creatinine concentration levels, etc. As yet another illustrative example, continuous analyte sensor(s) 202 may include a multi-analyte sensor configured to measure glucose concentration levels, lactate concentration levels, creatinine concentration levels, etc.

Accordingly, continuous analyte sensor(s) 202 is configured to generate at least one analog electrical signal that is proportional to the concentration level of a particular analyte, and sensor electronics module 204 is configured to convert the analog electrical signal into an analyte sensor count values, calibrate the analyte sensor count values based on the sensitivity profile of the continuous analyte sensor(s) 202 to generate measured analyte concentration levels, and transmit the measured analyte concentration level data, including the measured analyte concentration levels, to a display device, such as display devices 210, 220, 230, and/or 240, via a wireless connection. For example, sensor electronics module 204 may be configured to sample the analog electrical signal at a particular sampling period (or rate), such as every 1 second (1 Hz), 5 seconds, 10 seconds, 30 seconds, 1 minute, 3 minutes, 5 minutes, etc., and to transmit the measured analyte concentration data to the display device at a particular transmission period (or rate), which may be the same as (or longer than) the sampling period, such as every 1 minute (0.016 Hz), 5 minutes, 10 minutes, 30 minutes, at the conclusion of the wear period, etc. Depending on the sampling and transmission periods, the measured analyte concentration data transmitted to the display device include at least one measured analyte concentration level having an associated time tag, sequence number, etc.

In certain embodiments, continuous analyte sensor(s) 202 incorporates a thermocouple to provide an analog temperature signal to the sensor electronics module 204. The temperature signal can be used to correct the analog electrical signal or the measured analyte data for temperature. The thermocouple is implemented in various alternative but equally compatible methods. It can be included in the sensing region, incorporated into the sensor electronics module 204, or, contact the epidermis of the patient through openings in the adhesive pad.

The sensor electronics module 204 includes, inter alia, processor 233, storage element or memory 234, wireless transmitter/receiver (transceiver) 236, one or more antennas coupled to wireless transceiver 236, analog electrical signal processing circuitry, analog to-digital (A/D) signal processing circuitry, digital signal processing circuitry, a power source for continuous analyte sensor(s) 202 (such as a potentiostat), etc.

Processor 233 may be a general-purpose or application-specific microprocessor, an application-specific integrated circuit (ASIC), a field programmable gate array (FPGA), etc., that executes instructions to perform control, computation, input/output, etc. functions for the sensor electronics module 204. Processor 233 may include a single integrated circuit, such as a micro processing device, or multiple integrated circuit devices and/or circuit boards working in cooperation to accomplish the appropriate functionality. In certain embodiments, processor 233, memory 234, wireless transceiver 236, the A/D signal processing circuitry, and the digital signal processing circuitry are combined into a system-on-chip (SoC).

Generally, processor 233 may be configured to sample the analog electrical signal using the A/D signal processing circuitry at regular intervals (such as the sampling period) to generate analyte sensor count values based on the analog electrical signals produced by the continuous analyte sensor(s) 202, calibrate the analyte sensor count values based on the sensitivity profile of the continuous analyte sensor(s) 202 to generate measured analyte concentration levels, and generate measured analyte data from the measured analyte concentration levels, generate sensor data packages that include, inter alia, the measured analyte concentration level data. Processor 233 may store the measured analyte concentration level data in memory 234, and generate the sensor data packages at regular intervals (such as the transmission period) for transmission by wireless transceiver 236 to a display device, such as display devices 210, 220, 230, and/or 240. Processor 233 may also add additional data to the sensor data packages, such as supplemental sensor information that includes a sensor identifier, a sensor status, temperatures that correspond to the measured analyte data, etc. The sensor data packages are then wirelessly transmitted over a wireless connection to the display device. In certain embodiments, the wireless connection is a Bluetooth or Bluetooth Low Energy (BLE) connection. In such embodiments, the sensor data packages are transmitted in the form of Bluetooth or BLE data packets to the display device

Memory 234 includes volatile and nonvolatile medium. Memory 234 can include combinations of random access memory (RAM), dynamic RAM (DRAM), static RAM (SRAM), read only memory (ROM), flash memory, cache memory, and/or any other type of non-transitory computer-readable medium. Memory 234 is stores one or more analyte sensor system applications, modules, instruction sets, etc. for execution by processor 233, such as instructions to generate measured analyte data from the analyte sensor count values, etc.

Additionally or alternatively, Memory 234 stores certain sensor operating parameters 235, such as a calibration slope (or calibration sensitivity), a calibration baseline, etc. In particular, in certain implementations, information such as the calibration sensitivity, calibration baseline, and other information related to the sensitivity profile for the continuous analyte monitoring systems 104 are programmed into the sensor electronics module 204 during the manufacturing process, and then used to convert the analyte sensor electrical signals into measured analyte concentration levels. For example, as discussed above, the calibration slope is used to predict an initial in vivo sensitivity (M₀) and a final in vivo sensitivity (M_{f}), which are stored in memory 234 and used to convert the analyte sensor electrical signals into measured analyte concentration levels. In certain embodiments, calibration sensitivity (M_{CC}) 246 and/or calibration baseline 247 may be stored in memory 234.

In certain embodiments, sensor electronics module 204 can be physically connected to continuous analyte sensor(s) 202 and can be integral with (non-releasably attached to) or releasably attachable to continuous analyte sensor(s) 202. Sensor electronics module 204 includes hardware, firmware, and/or software that enable measurement of levels of analyte(s) via continuous analyte sensor(s) 202. For example, sensor electronics module 204 can include a potentiostat, a power source for providing power to the sensor, other components useful for signal processing and data storage, and a telemetry module for transmitting data from the sensor electronics module to, e.g., one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor.

Display devices 210, 220, 230, and/or 240 are configured for displaying displayable sensor data, including analyte data, which may be transmitted by sensor electronics module 204. Display devices 210, 220, 230, or 240, can be implemented as aa touchscreen display 212, 222, 232, and/or 242 for displaying sensor data to a patient and/or for receiving inputs from the patient. For example, a graphical user interface (GUI) may be presented to the patient for such purposes. In certain embodiments, one or more of the display devices 210, 220, 230, and 240 include other types of user interfaces such as a voice user interface instead of, or in addition to, a touchscreen display for communicating sensor data to the patient of the display device and/or for receiving patient inputs. Display devices 210, 220, 230, and 240 are examples of display device 107 illustrated in FIG. 1 used to display sensor data to a patient of the system of FIG. 1 and/or to receive input from the patient.

In certain embodiments, one, some, or all of the display devices are configured to display or otherwise communicate (e.g., verbalize) the sensor data as it is communicated from the sensor electronics module (e.g., in a customized data package that is transmitted to display devices based on their respective preferences), without any additional prospective processing required for calibration and real-time display of the sensor data.

The plurality of display devices may include a custom display device specially designed for displaying certain types of displayable sensor data associated with analyte data received from sensor electronics module. In certain embodiments, the plurality of display devices are configured for providing alerts/alarms based on the displayable sensor data. Display device 210 is an example of such a custom device. In certain embodiments, one of the plurality of display devices is a smartphone, such as display device 220 which represents a mobile phone, using a commercially available operating system (OS), and configured to display a graphical representation of the continuous sensor data (e.g., including current and historic data). Other display devices can include other hand-held devices, such as display device 230 which represents a tablet, display device 240 which represents a smart watch or fitness tracker, medical device 208 (e.g., an insulin delivery device), and/or a desktop or laptop computer (not shown).

Because different display devices provide different user interfaces, content of the data packages (e.g., amount, format, and/or type of data to be displayed, alarms, and the like) can be customized (e.g., programmed differently by the manufacture and/or by an end patient) for each particular display device. Accordingly, in certain embodiments, a plurality of different display devices can be in direct wireless communication with a sensor electronics module (e.g., such as an on-skin sensor electronics module 204 that is physically connected to continuous analyte sensor(s) 202) during a sensor session to enable a plurality of different types and/or levels of display and/or functionality associated with the displayable sensor data.

As mentioned, sensor electronics module 204 may be in communication with a medical device 208. Medical device 208 is a passive device in some example embodiments of the disclosure. Medical device 208 may be an insulin pump for administering insulin to a patient. For a variety of reasons, it may be desirable for such an insulin pump to receive and track glucose values transmitted from continuous analyte monitoring systems 104, where continuous analyte sensor 202 is configured to measure at least glucose. In another example, medical device 208 may be a CPAP machine which may function as an indirect calorimeter. If the patient uses a CPAP machine on a daily basis, utilizing a CPAP machine may be beneficial to gather indirect calorimetry readings in order to monitor for changes in liver metabolic function (e.g., as a function of overall metabolic function measured by indirect calorimetry).

Further, in some implementations of continuous analyte monitoring system 104, sensor electronics module 204 is in communication with other non-analyte sensors 206. Non-analyte sensors 206 may include, but are not limited to, an altimeter sensor, an accelerometer sensor, a global positioning system (GPS) sensor, a temperature sensor, a respiration rate sensor, etc. Non-analyte sensors 206 may also include monitors such as heart rate monitors, blood pressure monitors, pulse oximeters, caloric intake monitors, indirect calorimetry devices, photoplethysmography devices, and medicament delivery devices. One or more of these non-analyte sensors 206 may provide data to therapy management engine 114 described further below. In some aspects, a patient may manually provide some of the data for processing by training server system 140 and/or therapy management engine 114 of FIG. 1.

In certain embodiments, non-analyte sensors 206 further include sensors for measuring skin temperature, core temperature, sweat rate, and/or sweat composition.

In certain embodiments, the non-analyte sensors 206 can be combined in any other configuration, such as, for example, combined with one or more continuous analyte sensors 202. As an illustrative example, a non-analyte sensor, e.g., a temperature sensor, may be combined with a continuous glucose sensor 202 to form a glucose/temperature sensor used to transmit sensor data to the sensor electronics module 204 using common communication circuitry.

In certain embodiments, a wireless access point (WAP) is used to couple one or more of continuous analyte monitoring system 104, the plurality of display devices, medical device(s) 208, and/or non-analyte sensor(s) 206 to one another. In such embodiments, WAP 138 can provide Wi-Fi and/or cellular connectivity among these devices.

In certain embodiments, one or more of a variety of short-range communication protocols may be used for wireless communication among devices depicted in diagram 200 of FIG. 2. For example, in some embodiments, the one or more of continuous analyte monitoring system 104, the plurality of display devices, medical device(s) 208, and/or non-analyte sensor(s) 206 communicate with one another using Near Field Communication (NFC) and/or Bluetooth/BLE protocols.

FIG. 3 illustrates example inputs and example metrics that are calculated based on the inputs for use by the therapy management system of FIG. 1, according to some embodiments disclosed herein. In particular, FIG. 3 provides a more detailed illustration of example inputs and example metrics introduced in FIG. 1.

FIG. 3 illustrates example inputs 128 on the left, application 106 and DAM 116 in the middle, and metrics 130 on the right. In certain embodiments, each one of metrics 130 correspond to one or more values, e.g., discrete numerical values, ranges, or qualitative values (high/medium/low, stable/unstable, etc.). Application 106 obtains inputs 128 through one or more channels (e.g., manual patient input, sensors, other applications executing on display device 107, an EMR system, etc.). As mentioned previously, in certain embodiments, inputs 128 are processed by DAM 116 to output a plurality of metrics, such as metrics 130. Inputs 128 and metrics 130 can be used by training server system 140 and therapy management engine 114 to both train and deploy one or more machine learning models for classifying patients, predicting the disease state of a patient, and other functionalities described herein.

In certain embodiments, starting with inputs 128, patient statistics, such as one or more of age, height, weight, BMI, body composition (e.g., % body fat), stature, build, or other information are also provided as an input. In certain embodiments, patient statistics are provided through a patient interface, by interfacing with an electronic source such as an electronic medical record, and/or from measurement devices. In certain embodiments, the measurement devices include one or more of a wireless, e.g., Bluetooth-enabled, weight scale and/or camera, which may, for example, communicate with the display device 107 to provide patient data.

In certain embodiments, treatment/medication information is also provided as an input. Medication information may include information about the type, dosage, and/or timing of when one or more medications are to be taken by the patient. Treatment information may include information regarding different lifestyle habits recommended by the patient's physician. For example, the patient's physician may recommend a patient follow specific diet recommendations, exercise at a specific time during the day for a specific duration, or cut calories by 500 to 1,000 calories daily to improve glucose levels and therefore improve disease state. In certain embodiments, treatment/medication information is provided through manual patient input.

In certain embodiments, analyte sensor data is also provided as input, for example, through continuous analyte monitoring system 104. In certain embodiments, analyte sensor data includes glucose data measured by at least a glucose sensor (or multi-analyte sensor) in continuous analyte monitoring system 104.

In certain embodiments, input is also received from one or more non-analyte sensors, such as non-analyte sensors 206 described with respect to FIG. 2. Input from such non-analyte sensors 206 may include information related to a heart rate, a respiration rate, oxygen saturation, blood pressure, or a body temperature (e.g. to detect illness, physical activity, etc.) of a patient. In certain embodiments, electromagnetic sensors also detect low-power radio frequency (RF) fields emitted from objects or tools touching or near the object, which may provide information about patient activity or location.

In certain embodiments, input received from non-analyte sensors includes input relating to a patient's insulin delivery. In particular, input related to the patient's insulin delivery may be received, via a wireless connection on a smart pen, via patient input, and/or from an insulin pump. Insulin delivery information may include one or more of insulin volume, time of delivery, etc. Other parameters, such as exogenous insulin action time or duration of exogenous insulin action, may also be received as inputs.

In certain embodiments, starting with inputs 128, food consumption information includes information about one or more of meals, snacks, and/or beverages, such as one or more of the size, content (carbohydrate, fat, protein, etc.), sequence of consumption, and time of consumption. In certain embodiments, food consumption is provided by a patient through manual entry, by providing a photograph through an application that is configured to recognize food types and quantities, and/or by scanning a bar code or menu. In various examples, meal size may be manually entered as one or more of calories, quantity (“three cookies”), menu items (“Royale with Cheese”), and/or food exchanges (1 fruit, 1 dairy). In some examples, meal information may be received via a convenient user interface provided by application 106.

In certain embodiments, food consumption information (the type of food (e.g., liquid or solid, snack or meal, etc.) and/or the composition of the food (e.g., carbohydrate, fat, protein, etc.)) is determined automatically based on information provided by one or more sensors. Some example sensors may include body sound sensors (e.g., abdominal sounds may be used to detect the types of meal, e.g., liquid/solid food, snack/meal, etc.), radio-frequency sensors, cameras, hyperspectral cameras, and/or analyte (e.g., insulin, glucose, lactate, etc.) sensors to determine the type and/or composition of the food.

In certain embodiments, medical history and/or disease diagnoses (e.g., liver disease, diabetes, kidney disease, hypertension, etc.) is provided as an input. For example, the patient may have an existing diagnosis of liver disease and/or diabetes and this diagnosis may be provided through manual patient input. In certain embodiments, disease diagnoses are also provided by interfacing with an electronic source such as an electronic medical record.

In certain embodiments, time are also provided as an input, such as time of day or time from a real-time clock. For example, in certain embodiments, input analyte data is timestamped to indicate a date and time when the analyte measurement was taken for the patient.

Patient input of any of the above-mentioned inputs 128 may be provided through continuous analyte sensor system 104, non-analyte sensors 206, and/or a user interface, such a user interface of display device 107 of FIG. 1. As described above, in certain embodiments, DAM 116 determines or computes the patient's metrics 130 based on inputs 128. An example list of metrics 130 is shown in FIG. 3.

In certain embodiments, glucose metrics are determined from sensor data (e.g., blood glucose measurements obtained from a continuous glucose sensor of continuous analyte monitoring system 104). For example, glucose metrics refer to time-stamped glucose measurements or values that are continuously generated and stored over time. In some examples, glucose metrics may also be determined, for example, based upon historical data in particular situations, e.g., given a combination of food consumption, insulin, and/or exercise.

In certain embodiments, a minimum and maximum glucose level are determined from sensor data. For example, a daily minimum and maximum glucose values for each day over a specified amount of time (e.g., a week or a month) may be determined. In certain embodiments, the minimum and maximum glucose levels are determined based on an average minimum and maximum over a specified amount of time (e.g., a week or a month). In certain embodiments, DAM 116 continuously or periodically calculates a normal glucose range and time-stamp and store the corresponding information in the patient's profile 118.

In other embodiments, a normal minimum and maximum glucose level may be determined from population data (e.g., from data records or historical patients with liver disease and/or diabetes). In such embodiments, each patient may have personalized, customized, acceptable glucose minimum and/or maximum glucose values, which may be determined based on time periods when the patient is in a fasting state or during a meal, for example.

In certain embodiments, a glucose baseline is determined from sensor data (e.g., blood glucose measurements obtained from a continuous glucose sensor of continuous analyte monitoring system 104). A glucose baseline represents a patient's normal glucose levels during periods where fluctuations in glucose production is typically not expected. A patient's baseline glucose level is generally expected to remain constant over time, unless challenged through an action such as consuming food or exercise by the patient, for example. Additionally, a patient's baseline glucose level may also change based on the patient's health, specifically an improvement or decline in liver health and/or diabetes. Further, each patient may have a different glucose baseline. In certain embodiments, a patient's glucose baseline is determined by calculating an average of glucose levels over a specified amount of time where fluctuations are not expected.

For example, the baseline glucose level for a patient may be determined over a period of time when the patient is sleeping, sitting in a chair, or other periods of time where the patient is sedentary and not consuming food or medication which would reduce or increase glucose levels. In certain embodiments, DAM 116 continuously, semi-continuously, or periodically calculates a glucose baseline and time-stamp and store the corresponding information in the patient's profile 118. In certain embodiments, DAM 116 calculates the glucose baseline using glucose levels measured over a period of time where the patient is sedentary, the patient is not consuming glucose-heavy foods, and where no external conditions exist that would affect the glucose baseline. In certain other embodiments, DAM 116 may use glucose levels measured over a period of time where the patient is, at least for a subset of the period of time, engaging in exercise and/or consuming glucose and/or an external condition exists that would affect the glucose baseline level. In this case, in some examples, DAM 116 may first identify which measured glucose values are to be used for calculating the baseline glucose level by identifying glucose values that may have been affected by an external event, such the consumption of food, exercise, medication, or other perturbation that would disrupt the capture of a glucose baseline measurement. DAM 116 may then exclude such measurements when calculating the glucose baseline level of the patient. In some other examples, DAM 116 may calculate the glucose baseline level by first determining a percentage of the number of glucose values measured during a specific time period that represent the lowest glucose values measured. DAM 116 may then take an average of this percentage to determine the glucose baseline level.

In certain embodiments, a glucose rate of change is determined from glucose data (e.g., blood glucose measurements obtained from a continuous glucose sensor of continuous analyte monitoring system 104). A glucose rate of changes refers to a rate that indicates how one or more time-stamped glucose measurements or values change in relation to one or more other time-stamped glucose measurements or values. Glucose rates of change may be determined over one or more seconds, minutes, hours, days, etc. Further, glucose rate of change may be positive, negative, or an absolute value.

In certain embodiments, a post-prandial glucose dynamic is determined from glucose data (e.g., blood glucose measurements obtained from a continuous glucose sensor of continuous analyte monitoring system 104). A post-prandial glucose dynamic may refer to glucose trends following the consumption of food or a meal. For example, the post-prandial glucose dynamic may be the peak of the patient's post-prandial glucose spike (e.g., post-prandial glucose spike maximum), the duration of hyperglycemia (above baseline) over a set time period following a meal, and/or rate of change of glucose as the patient's glucose level returns to baseline. Post-prandial glucose dynamics may be determined on a daily basis over a set time period following a meal, and may be averaged over a second time period (e.g., a week or a month) to monitor for changes in post-prandial glucose dynamics over time.

In certain embodiments, a nocturnal hypoglycemia pattern is determined from glucose data (e.g., blood glucose measurements obtained from a continuous glucose sensor of continuous analyte monitoring system 104). A nocturnal hypoglycemia pattern may refer to a nighttime glucose level below the patient's daytime glucose baseline level. For example, nocturnal hypoglycemia may be determined where the patient's nighttime glucose level is a delta from the patient's daytime baseline glucose levels, or a population's day time baseline glucose levels (e.g., 20 mg/dL below the patients day time baseline, for example).

In certain embodiments, a dawn effect pattern is determined from glucose data (e.g., blood glucose measurements obtained from a continuous glucose sensor of continuous analyte monitoring system 104). A dawn effect pattern may refer to an increase in a patient's glucose level in the morning, as the patient wakes from sleep. For example, a dawn effect may be determined when the patient's glucose level spikes to a level of more than 120 mg/dL without cause (e.g., not in response to consumption of a meal or an exercise session, for example). In another example, a dawn effect may be determined when the patient's glucose level increases more than 30 mg/dL from the time of waking without cause (e.g., if a patient's glucose is 70 mg/dL at the time of waking and increases to 100 mg/dL without cause). Further, a dawn effect pattern may be based on the time of day the glucose level spike occurs. For example, DAM 116 may determine the time of day of the glucose spike to determine whether the glucose spike without cause may be attributed to the dawn effect.

In certain embodiments, insulin sensitivity is determined using historical data, real-time data, or a combination thereof, and may, for example, be based upon one or more inputs 128, such as one or more of food consumption information, continuous analyte sensor data, non-analyte sensor data (e.g., insulin delivery information from an insulin device), etc. Insulin sensitivity refers to how responsive a patient's cells are to insulin. Improving insulin sensitivity for a patient may help to reduce insulin resistance in the patient.

In certain embodiments, health and sickness metrics are determined, for example, based on one or more of patient input (e.g., pregnancy information or known sickness information), from physiologic sensors (e.g., temperature), activity sensors, or a combination thereof. In certain embodiments, based on the values of the health and sickness metrics, for example, a patient's state is defined as being one or more of healthy, ill, rested, or exhausted.

In certain embodiments, the meal state metric indicates the state the patient is in with respect to food consumption. For example, the meal state may indicate whether the patient is in one of a fasting state, pre-meal state, eating state, post-meal response state, or stable state. In certain embodiments, the meal state also indicates nourishment on board, e.g., meals, snacks, or beverages consumed, and may be determined, for example from food consumption information, time of meal information, and/or digestive rate information, which may be correlated to food type, quantity, and/or sequence (e.g., which food/beverage was eaten first).

In certain embodiments, meal habits metrics are based on the content and the timing of a patient's meals. For example, if a meal habit metric is on a scale of 0 to 1, the better/healthier the meal consumed by the patient, the higher the meal habit metric of the patient will be to 1, in an example. Better/healthier meals may be defined as those that do not drive glucose levels of a patient out of a normal glucose range for the patient (e.g., 70-180 mg/dL or the patient's desired range). Also, the more the patient's food consumption adheres to a certain time schedule, the closer their meal habit metric will be to 1, in the example. In certain embodiments, the meal habit metrics reflects the contents of a patient's meals where, e.g., three numbers may indicate the percentages of carbohydrates, proteins and fats.

In certain embodiments, medication habit metrics are based on the patient's prescribed medications and a determination of whether the prescribed medications may have an effect on the patient's analyte levels. For example, by analyzing a patient's medication habits, DAM 116 may determine whether the patient's medications may impact the patient's analyte measurements at a particular time. Based on the patient's medication habits, DAM 116 may determine whether the patient's analyte levels are a result of medication consumption or worsening liver function, for example. Medication habit metrics may be time-stamped so that they can be correlated with the patient's analyte levels at the same time.

In certain embodiments, medication adherence is measured by one or more metrics that are indicative of how committed the patient is towards their medication regimen. In certain embodiments, medication adherence metrics are calculated based on one or more of the timing of when the patient takes medication (e.g., whether the patient is on time or on schedule), the type of medication (e.g., is the patient taking the right type of medication), and the dosage of the medication (e.g., is the patient taking the right dosage).

In certain embodiments, the activity level metric indicates the patient's level of activity. In certain embodiments, the activity level metric is determined, for example based on input from an activity sensor or other physiologic sensors, such as non-analyte sensors 206. In certain embodiments, the activity level metric may be calculated by DAM 116 based on one or more of inputs 128, such as one or more of exercise information, non-analyte sensor data (e.g., accelerometer data), time, patient input, etc. In certain embodiments, the activity level is expressed as a step rate of the patient. Activity level metrics may be time-stamped so that they can be correlated with the patient's glucose metrics at the same time.

In certain embodiments, activity intensity level metrics indicate the intensity level with which the patient is performing the activity. For example, activity intensity level metrics may include information indicating that the patient is engaging in low intensity physical activity, low-to-moderate intensity physical activity, moderate intensity physical activity, moderate-to-high intensity physical activity, and/or high intensity physical activity, which may all impact the patient's glucose metrics. In certain embodiments, activity intensity level metrics are calculated by DAM 116 based on one or more of inputs 128, such as one or more of physical activity information, non-analyte sensor data, time, patient statistics, etc. For example, in certain embodiments, activity intensity level metrics are determined based on physical activity information, such as input from an activity sensor on a fitness tracker or other physiologic sensors. In certain embodiments, activity intensity level metrics are determined based on input from other non-analyte sensors, such as an accelerometer, exercise equipment sensor (e.g., a power meter), GPS device, heart rate monitor, EKG device, EMG device, respiration monitor, temperature monitor, blood pressure monitor, pulse oximeter, etc. In certain embodiments, activity intensity level metrics are determined based on skin temperature, core temperature, sweat rate, and/or sweat composition. In certain embodiments, activity intensity level metrics are determined based on patient statistics, such as information stored in patient profile 118 or provided through manual patient input. In further embodiments, activity level metrics may be based on continuous analyte sensor data measured by continuous analyte sensor(s) 202, such as glucose metrics.

In certain embodiments, exercise regimen metrics indicate one or more of the type of activities the patient engages in, the corresponding intensity of such activities, frequency the patient engages in such activities, etc. In certain embodiments, exercise regimen metrics are calculated based on one or more of analyte and/or non-analyte sensor data input (e.g., non-analyte sensor data input from an accelerometer, a heart rate monitor, a blood pressure monitor, a respiration rate sensor, etc.), calendar input, patient input, etc.

In certain embodiments, body temperature metrics are calculated by DAM 116 based on inputs 128, and more specifically, non-analyte sensor data from a temperature sensor. In certain embodiments, heart rate metrics are calculated by DAM 116 based on inputs 128, and more specifically, non-analyte sensor data from a heart rate sensor. In certain embodiments, respiratory rate metrics are calculated by DAM 116 based on inputs 128, and more specifically, non-analyte sensor data from a respiratory rate sensor.

### Example Methods for Predicting a Patient's Disease State Using Continuously Monitored Analyte Data

FIG. 4 illustrates a flow diagram of an example method 400 for classifying a patient as a healthy patient, a patient with liver disease, or a diabetic patient, predicting a current or future diabetes or liver disease state of a patient, and providing recommendations to the patient based on the predicted disease state using at least glucose measurements. Method 400 may be performed by therapy management system 100 to collect data, including for example, analyte data generated by a continuous analyte monitoring system 104, patient information, and non-analyte sensor data mentioned above, to (1) classify the patient as a healthy patient, a diabetic patient, or a patient with liver disease; (2) predict a current or future disease state of the patient; and (3) provide recommendations to the patient for treatment to improve the patient's liver disease and/or diabetes disease state. In other words, therapy management engine 114 presented herein may offer information to diagnose and stage liver disease and/or diabetes and provide a recommendation to a patient to improve their disease state. Method 400 is described below with reference to FIGS. 1 and 2 and their components.

At block 402, method 400 begins by therapy management engine 114 receiving analyte data from continuous analyte monitoring system 104, and continuous analyte sensor(s) 202 illustrated in FIG. 2. For example, continuous analyte monitoring system 104 may comprise a continuous glucose sensor 202 to measure the patient's analyte levels, such as glucose levels. Further, therapy management engine 114 may receive data from patient inputs. The patient inputs may be received in a variety of ways. For example, the inputs may be received or retrieved from the patient profile 118, which includes demographic info 120, disease info 122, and medication info 124, inputs 128, metrics 130, etc. Inputs may also be received as patient input through the patient interface of a display device 107.

At block 404, therapy management engine 114 may classify the patient as a healthy patient, a patient with liver disease, or a diabetic patient based on the analyte data from continuous analyte monitoring system and received inputs. A patient may be classified at block 404 using a variety of models, such as a rules-based model, a machine learning model, or the like.

In embodiments where a rules-based model is used, the patient's inputs and analyte data may be mapped to a certain classification using, for example, a rules library. For example, the rules-based model may take inputs received at block 402 and classify the patient into a healthy patient (and therapy management engine 114 may proceed to block 406), a patient with liver disease (and therapy management engine 114 may proceed to block 408), or a diabetic patient (and therapy management engine 114 may proceed to block 410).

An example rule may include classifying a patient as a healthy patient if the patient self-identified as a healthy patient (e.g., the patient does not report a liver disease or diabetes diagnosis). Another example rule may classify the patient as a healthy patient if the analyte data indicates that the patient has average glucose fluctuations, average delta from baseline (e.g., the average change from baseline glucose levels when the patient is experiencing a post-prandial glucose spike, or when the patient is experiencing nocturnal hypoglycemia, for example), and/or average glucose baseline when compared to historical population data of healthy patients. In other words, for example, if the patient's average glucose fluctuations are similar to the average glucose fluctuations of other healthy patients, then that might be an indication in favor of classifying the patient as a healthy patient. Any combination of rules, including the exemplary rules described above, may be used for classifying a patient as a healthy patient.

In certain other embodiments, a patient may be classified as a patient with liver disease if the patient self-identified as a patient with liver disease, or reports a known diagnosis of liver disease, for example. Another example rule may classify the patient as a patient with liver disease if the analyte data indicates that the patient has average glucose fluctuations, average delta from baseline (e.g., the average change from baseline glucose levels when the patient is experiencing a post-prandial glucose spike, or when the patient is experiencing nocturnal hypoglycemia, for example), and/or average glucose baseline similar to a historical population data of patients with liver disease. In other words, for example, if the patient's average glucose fluctuations are similar to the average glucose fluctuations of other patients with liver disease, then that might be an indication in favor of classifying the patient as a patient with liver disease. Any combination of rules, including the exemplary rules described above, may be used for classifying a patient as a patient with liver disease.

In certain embodiments, a patient is classified as a diabetic patient if the patient self-identified as a diabetic patient, or reports a known diagnosis of diabetes, for example. Another example rule may include classifying the patient as a diabetic patient if the analyte data indicate that the patient has average glucose fluctuations, average delta from baseline (e.g., the average change from baseline glucose levels when the patient is experiencing a post-prandial glucose spike, or when the patient is experiencing nocturnal hypoglycemia, for example), and/or average glucose baseline similar to a historical population data of diabetic patients. In other words, for example, if the patient's average glucose fluctuations are similar to the average glucose fluctuations of other diabetic patients, then that might be an indication in favor of classifying the patient as a patient with diabetes. Any combination of rules, including the exemplary rules described above, may be used for classifying a patient as a patient with diabetes.

In certain embodiments, the rules are more granular, such that a combination of rules and a variety of inputs are used in determining a classification. An example of such a rule is to classify the patient as a patient with liver disease if the inputs and analyte data show that the patient has (1) a medical history noting a risk of developing liver disease, (2) an increase in baseline glucose level of 10 mg/dL over 2 weeks, and (3) a post-prandial glucose spike more than 75 mg/dL above the patient's baseline glucose level. In another example, therapy management engine 114 may classify the patient as a diabetic patient if inputs and analyte data show that the patient has an increase in baseline glucose level over time (e.g., more than 100 mg/dL) in combination with normal night time glucose values (e.g., not more than 30 mg/dL less that day time baseline glucose levels).

In certain embodiments, instead of a rules-based model, an AI/ML (interchangeably referred to as an “ML model” for simplicity) model is used to predict the patient's classification. For example, some or all of the inputs received at block 402 may be used as input to the model that is trained to classify a corresponding patient. In such cases, the model is trained using a training dataset, including historical population-based data of many patients, who have already been classified as a healthy patient, a patient with liver disease, or a diabetic patient. In such an example, the training dataset is labeled with such classifications. In certain embodiments, the output of the model is accompanied by a confidence score. In certain embodiments, if the confidence score is lower than a threshold, the therapy management engine 114 does not provide the predicted classification to the patient. As an example, a classification may be predicted with a low confidence score for a particular patient because the inputs to the ML model for the patient did not include glucose measurements. In such an example, a patient without a classification due to unknown glucose levels or otherwise, may be instructed to wear a continuous analyte monitor based on various comorbidities (e.g., hypertension, obesity, kidney disease, diabetes, etc.), age of the patient, a family history of liver disease or diabetes, a high HbA1C (e.g., high HbA1C, but not in the range of a diabetic patient), or an abnormal liver metabolic panel, for example. Once the glucose measurements are available for the patient, the ML model may use them as input for providing a classification with a higher confidence score.

Upon determining whether the patient is classified as a healthy patient, a patient with diabetes, or a liver disease patient, therapy management engine 114 uses various models (e.g., rules-based models or ML models) to (1) assess a healthy patient's risk of developing liver disease, (2) monitor a patient diagnosed with liver disease for liver disease progression and/or development of diabetes, and (3) determine whether a patient with diabetes also has liver disease. By following the appropriate method based on a patient's classification, therapy management engine 114 may accurately determine liver disease risk, liver disease diagnosis, and/or diabetes diagnosis of a patient. Additionally, based on the classification and the disease state, therapy management engine 114 may further provide recommendations to the patient regarding treatment to treat or prevent liver disease, to prevent the progression of liver disease, and/or to treat or prevent diabetes.

For example, if the patient is classified as a healthy patient, or if the patient is unclassified, then therapy management engine 114 proceeds to block 406 to monitor glucose metrics to diagnose liver disease, which the patient may develop in the future, or determine a risk factor for developing liver disease in the future. If the patient is classified as a patient with liver disease, then therapy management engine 114 proceeds to block 408 to monitor glucose metrics for the progression of liver disease and/or the presence or development of diabetes. If the patient is classified as a diabetic patient, then therapy management engine 114 proceeds to block 410 to monitor glucose metrics to detect the presence or monitor for the development of liver disease. In certain embodiments, therapy management engine 114 monitors the patient for development of liver disease, monitor the patient for progression of liver disease, assess liver disease risk, diagnose liver disease, and/or diagnose diabetes without a patient classification.

Determining a Healthy Patients Liver Disease State and/or Providing Therapy Management Recommendations to Improve Disease State

If a patient is classified as a healthy patient or the patient is unclassified at block 404, method 400 continues to block 406. At block 406, therapy management engine 114 may monitor the patient's glucose metrics using continuous analyte sensor 202. Continuous analyte data may, over time, allow therapy management engine 114 to provide a liver disease diagnosis or determine a risk factor for developing liver disease for a healthy patient (e.g., by monitoring liver metabolic function). In certain embodiments, therapy management engine 114 utilizes metrics (e.g., metrics 130) to determine a patient's liver disease state or risk of developing liver disease.

At block 412, therapy management engine 114 may provide a liver disease diagnosis or liver disease risk factor to the healthy patient based on monitored glucose metrics and other inputs. Therapy management engine 114 may utilize a rules-based model or an ML model to determine a patient's disease state (e.g., similar to the models patient herein for classification of a patient) and/or, as discussed in reference to block 418, provide therapy management recommendations to the patient to improve disease state. In an example of a rules-based model, various rules may be defined around a set of parameters. The patient's glucose metrics and metrics related to inputs (e.g., metrics 130) over time may be mapped to a certain disease state using, for example, a rules library based on certain parameters. For example, a rule may dictate that if the patient has a baseline glucose <100 mg/dL, the patient does not experience post-prandial glucose spikes (e.g., glucose level remains below 140 mg/dL), and the patient experiences stable glucose levels throughout the night, the therapy management engine 114 may continue to classify the patient as a healthy patient (e.g., the patient does not have liver disease and/or diabetes). In other words, the liver disease diagnosis for such a patient may indicate no liver disease, and the liver disease risk factor may be very low or zero.

In certain other embodiments, instead of a rules-based model, a ML model may be used to determine the patient's disease state and/or provide therapy management recommendations to the patient to improve disease state. The ML model may be trained using training data, which may include historical data associated with one or more patients diagnosed with liver disease in various stages, one or more healthy patient s, one or more patient s with diabetes, and/or one or more patient s with both diabetes and liver disease. Using the trained ML model, analyte data and input received by therapy management engine 114 may be provided to the ML model and the model may output a prediction of the patient's diagnosis of liver disease, a liver disease stage, or a risk factor for developing liver disease.

As discussed herein, therapy management engine 114 (e.g., using a rules-based or ML model) may be configured to analyze a patient's analyte data and inputs both retrospectively (e.g., provide feedback to a patient following a period of time based on patient averages and/or population data), and/or in real time (e.g., as the patient's glucose is measured, after therapy management system 100 has collected some historical data for the patient to establish expected glucose measurements). For example, therapy management engine 114 may analyze data from a time frame (e.g., 2 weeks, a month, a year, or more than a year) to determine average trends over time and provide disease diagnosis and/or treatment recommendations. In another example, therapy management engine 114 may collect data on the patient over time to establish a baseline for the patient. If the patient demonstrates higher baseline glucose level from expected baseline glucose, higher or lower post-prandial glucose spike maximum, etc., therapy management engine 114 may provide real time feedback to the patient that a specific event (e.g., exercise, meal, etc.) caused an excursion from expected glucose levels. In both cases, therapy management engine 114 is able to provide a disease state prediction as well as recommendations for future meals, medications, or exercise sessions to avoid glucose spikes, maintain glucose levels over night, etc.

Whether therapy management engine 114 utilizes a rules-based model or an ML model, therapy management engine 114 determines whether the patient has liver disease or is at risk of developing liver disease based on metrics, including, but not limited to, glucose metrics. For example, based on the patient's historical or current glucose data or population data, therapy management engine 114 may analyze various glucose metrics, including post-prandial glucose dynamics (e.g., the peak of the patient's post-prandial glucose spike, duration of hyperglycemia above baseline over a set time period post meal, rate of change of glucose as the patient's glucose returns to baseline), elevated baseline glucose, and nocturnal hypoglycemia, as described herein to determine if the patient has liver disease or is at risk of developing liver disease.

In certain embodiments, a healthy patient's post-prandial glucose dynamics demonstrates a small increase in glucose levels, followed by a decline in glucose levels back to baseline glucose levels within 2 hours following a meal. Therapy management engine 114 recognizes patterns in the patient's post-prandial glucose dynamics over time that demonstrate a worsening of liver health and/or development of liver disease. For example, therapy management engine 114 may determine the patient has liver disease or is at risk of developing liver disease based on whether, at some time point, the patient's average glucose level following a meal is 120 mg/dL, and at some later time point the patient's average glucose level following a meal is 150 mg/dL.

In another example, if a patient's return to baseline glucose level occurs more than 2 hours following a meal, therapy management engine 114 may determine that the patient has liver disease or is at risk of developing liver disease. However, return to baseline glucose levels following a meal may be dependent on the type of meal the patient consumed, the frequency and size of meals the patient consumes throughout the day, the patient's metabolic condition, and any medication the patient may consume. To improve a patient's time to return to baseline following a meal, therapy management engine 114 may determine the patient's meal habits, medication consumption, and/or the patient's medical history based on patient inputs and determine the patient's average time to return to baseline following a post-prandial glucose spike. Based on the data collected from patient inputs, therapy management engine 114 may recommend changes to decrease the time for the patient to return to baseline glucose following a post-prandial glucose spike.

In order to determine the time it takes for the patient's glucose levels to return to baseline, therapy management engine 114 may suggest the patient consume a specific food composed of simple sugars (e.g., a glass of orange juice), and monitor the patient's glucose levels until the patient's glucose levels to return to baseline. Following consumption of the suggested food, therapy management engine 114 may instruct the patient not to exercise and not to consume any further food until the patient's glucose levels return to baseline level.

Over time, therapy management engine 114 may suggest the patient repeat this process (e.g., consumption of orange juice and therapy management engine 114 monitors the patient's glucose levels for return to baseline glucose levels) and therapy management engine 114 may analyze the amount of time it takes for the patient's glucose to return to baseline levels with over time. The analysis may include comparing the most recent one or more measurements of the amount of time it took for the patient's glucose to return to baseline levels with previous measurements recorded for the patient. Therapy management engine 114 may determine that the patient has liver disease or is at risk of developing liver disease based on the amount of time required for the patient's glucose levels to return to baseline increasing over time (e.g., at some time point the amount of time required for the patient's glucose levels to return to baseline is 2 hours, and at some later time point the time required in 3 hours).

Additionally, post-prandial glucose dynamics may include an area under the curve determination, which accounts for the magnitude of the glucose spike, the rate of change of glucose levels as glucose levels return to baseline, and the time to return to glucose baseline following a post-prandial glucose spike. A post-prandial glucose area under the curve may be different for patients depending on the presence and severity of liver disease and/or diabetes. For example, a healthy patient may have a less severe post-prandial glucose level spike following a meal and a faster glucose return to baseline following a meal (e.g., larger negative rate of change of glucose levels following a glucose spike). These factors contribute to a relatively small area under the curve when compared with a patient who has liver disease or diabetes. Therapy management engine 114 may then determine that the patient has liver disease or is at risk of developing liver disease based on a higher and/or increasing area under the curve over time.

In certain embodiments, nocturnal hypoglycemia demonstrates a patient's liver disease and/or risk of developing liver disease. For example, therapy management engine 114 may determine the patient is healthy (e.g., not developing or diagnosed with liver disease) based on glucose measurements that demonstrate a night time glucose level that has little to no delta from day time baseline glucose levels. Alternatively, therapy management engine 114 may determine the healthy patient's liver function is declining (e.g., the patient is at risk of developing liver disease) or the patient has developed liver disease based on the delta between the patient's night time glucose levels when compared to the patient's day time glucose levels increases over time, or if the patient's delta, when compared to population data of patients developing liver disease and/or diagnosed with liver disease, suggests the patient is developing or diagnosed with liver disease. In certain embodiments, a patient's nocturnal glucose level is indicative of a patient's fasting glucose levels. The patient's fasting glucose level is determined based on about 6 hours or more without food or other consumption of calories.

Over time, therapy management engine 114 may use the frequency, duration, and/or amplitude of the patient's nocturnal hypoglycemia to determine a risk of the patient developing liver disease. For example, if the patient regularly experiences mild nocturnal hypoglycemia (e.g., small delta from daytime baseline glucose levels) during the night, the patient may be considered at mild risk for developing liver disease. Alternatively, if the patient regularly experiences severe nocturnal hypoglycemia (e.g., large delta from daytime baseline glucose levels) during the night, the patient may be considered to be at high risk for developing liver disease. In certain embodiments, if the patient has a decreasing nocturnal glucose level, such as from 100 mg/dL to 70 mg/dL over time, the patient is considered to be at risk of developing liver disease or developing liver disease.

In certain embodiments, therapy management engine 114 determines a patient has liver disease or is at risk of developing liver disease based on fasting glucose in combination with one or more other metrics. For example, if a patient experiences a decreasing fasting glucose level and an increasing post-prandial glucose level, the patient may be determined to be at an increased risk of developing liver disease. In another example, a patient may be determined to have diabetes and not liver disease if the patient experiences high post-prandial glucose spikes but does not experience low fasting glucose levels. In another example, a low fasting glucose level alone, such as a fasting glucose level of 60 mg/dL, for a patient with a normal baseline glucose level of 100 mg/dL, may indicate a high risk of developing liver disease.

In certain embodiments, therapy management engine 114 recognizes an abnormal pattern in a patient's nighttime glucose levels while monitoring for nocturnal hypoglycemia. Therapy management engine 114 may determine the abnormal pattern is due to compression of the continuous analyte monitor (e.g., continuous glucose monitor) while the patient is sleeping. This compression may cause glucose values to appear lower than actual levels, which may lead to a determination that the patient is experiencing nocturnal hypoglycemia. However, if therapy management engine 114 determines the pattern, and therefore the lower glucose level, is due to compression, therapy management engine 114 may discard glucose measurements collected during the period of compression to avoid determining that the patient is experiencing nocturnal hypoglycemia.

In certain embodiments, the presence and/or severity of a dawn effect demonstrates a patient's liver disease and/or risk of developing liver disease. For example, therapy management engine 114 may determine the patient is healthy (e.g., not developing or diagnosed with liver disease) based on a lack of dawn effect, or a small glucose spike when compared to patients with a liver disease or diabetes diagnosis. Alternatively, therapy management engine 114 may determine the patient has liver disease or is at risk of developing liver disease based on the presence of a dawn effect comparable in magnitude and timing to historical patients having liver disease and/or developing liver disease, or if a patient's dawn effect magnitude and/or timing increases over time.

Additionally, when determining whether a healthy patient has liver disease or is at risk of developing liver disease, therapy management engine 114 may determine the patient's baseline glucose level and/or the change in a patient's baseline glucose level over time. For example, therapy management engine 114 may determine a patient is healthy based on glucose levels that demonstrate that the patient has a glucose baseline similar to a healthy patient population, or the patient's baseline glucose level is less than 100 mg/dL. Alternatively, therapy management engine 114 may monitor a patient's baseline glucose level over time to determine whether the patient's baseline glucose level is increasing, decreasing, or remaining stable over time. Therapy management engine 114 may then determine that the patient is developing liver disease or is at risk of developing liver disease based on the patient's baseline glucose levels increasing over time, among other factors described herein.

In certain embodiments, a daily minimum and maximum glucose level over time provides therapy management engine 114 with additional input in determining whether a healthy patient has liver disease or is at risk of developing liver disease. For example, a healthy patient may have a fluctuation between minimum and maximum daily glucose levels of around 90 mg/dL. However, therapy management engine 114 may determine that, over time, a patient's fluctuation between minimum and maximum daily glucose levels is increasing and/or exceeds 110 mg/dL. Based on the increasing fluctuation and/or fluctuation over 110 mg/dL, therapy management engine 114 may determine that the patient is developing liver disease or is at risk of developing liver disease.

In certain embodiments, daily minimum and maximum glucose levels are analyzed in light of the patient's lifestyle and/or activity level. For example, therapy management engine 114 may identify that the patient is completing an exercise session based on analyte sensor data and/or non-analyte sensor data, which may cause higher glucose levels following the exercise session than the patient would experience at rest. If therapy management engine 114 determines that the patient is exercising at some time point during the day and the patient's glucose level fluctuation is higher than average, therapy management engine 114 may not notify the patient of the increase in fluctuation and/or may not consider the glucose level fluctuation in a calculation for liver disease diagnosis over time. Additionally, when the patient is determined to be exercising, therapy management engine 114 may increase the threshold at which the glucose level fluctuation is determined to be indicative of liver disease or risk of liver disease.

In certain embodiments, therapy management engine 114 determines a maximum glucose level, without determining a minimum glucose level. For example, a healthy patient may be expected to have an average maximum glucose level around 140 mg/dL. However, therapy management engine 114 may determine that the patient has liver disease or is developing liver disease based on the patient's average maximum glucose level reaching more than 200 mg/dL and/or increasing over time.

Alternatively, therapy management engine 114 may determine a minimum glucose level, without determining a maximum glucose level. For example, a healthy patient may be expected to have an average minimum glucose level between 65-75 mg/dL. However, therapy management engine 114 may then determine that the patient has liver disease or is developing liver disease based on the patient's average minimum glucose level reaching 100 mg/dL or more and/or increasing over time.

In certain embodiments, therapy management engine 114 is configured to analyze a patient's analyte data and inputs and output a liver disease prediction and/or a liver disease risk score. In other words, therapy management engine 114 may analyze one or more of the above glucose metrics, in combination with patient inputs (e.g., inputs 128), to determine a liver disease prediction and/or a liver disease risk score for a healthy patient.

While the glucose metrics mentioned in reference to block 412 may assist therapy management engine 114 in determining the patient has liver disease and/or may be at risk for developing liver disease, the glucose metrics may also assist therapy management engine 114 to determine that the patient does not have liver disease and/or is not at risk of developing liver disease. For example, therapy management engine 114 may determine that the patient is healthy (e.g., does not have liver disease and/or is not at risk of developing liver disease) based on a patient not experiencing post-prandial glucose spikes, a patient experiencing mild post-prandial glucose spikes, and/or a patient's glucose level returns to baseline within two hours following a post-prandial glucose spike.

At block 418, therapy management engine 114 may provide recommendations for treatment to improve the patient's liver disease stage and/or risk of developing liver disease based on the patient's glucose metrics and the feedback provided to the patient at block 412. In response to various glucose metrics demonstrating a healthy patient is developing liver disease and/or is at risk of developing liver disease, therapy management engine 114 may recommend a treatment to address one or more glucose trends in order to prevent the development of liver disease or prevent the progression of liver disease.

For example, therapy management engine 114 may determine a patient suffers from mild nocturnal hypoglycemia at block 406 and determine that the patient is at risk of developing liver disease at block 412. At block 420, therapy management engine 114 may determine that medication and/or certain lifestyle changes may help the patient in maintaining glucose levels over night and reduce the incidence of nocturnal hypoglycemia. In order to prevent nocturnal hypoglycemia, therapy management engine 114 may suggest the patient consume a meal at a certain time in the evening, or consume a meal with specific macronutrients (e.g., consume a meal low in carbohydrates, for example). Additionally, therapy management engine 114 may also predict an ideal glucose level for the patient when the patient goes to sleep to ensure the patient will not experience nocturnal hypoglycemia overnight. If the patient's glucose level is not at or near the ideal glucose level for the patient when the patient goes to sleep, therapy management engine 114 may predict the patient will experience nocturnal hypoglycemia, and/or recommend the patient consume a small amount of food prior to going to sleep to avoid nocturnal hypoglycemia.

Further, in order to avoid nocturnal hypoglycemia, therapy management engine 114 may provide information to the patient on daytime glucose levels and patterns. If, based on the day time glucose patterns and/or non-analyte sensor data, therapy management engine 114 determines the patient has completed an exercise session that may lower the patient's glucose levels for a time period (e.g., Zone 2 aerobic exercise which causes increased energy expenditure for up to 48 hours) and/or the patient completed an exercise session prior to going to sleep, therapy management engine 114 may recommend the patient consume more carbohydrates before going to sleep or eat more carbohydrates throughout the day in an attempt to maintain glucose levels overnight.

In certain other embodiments, if the patient does not have diabetes, therapy management engine 114 may recommend a patient administer a fast-acting insulin bolus to lower glucose spikes throughout the day, including post-prandial glucose spikes.

In certain other embodiments, therapy management engine 114 may monitor a patient's glucose levels during the day and provide exercise-related feedback to address high maximum glucose levels and/or prevent high post-prandial glucose dynamics. For example, therapy management engine 114 may suggest the patient complete light exercise (e.g., a walk) following a large meal or prior to sleeping, if the patient's glucose level is too high prior to sleeping.

In still other embodiments, specifically if a healthy patient is beginning to develop liver disease and/or a patient is at risk of developing liver disease, therapy management engine 114 may instruct the patient to alter meal times (e.g., eat a light meal before bed), avoid exercising in the evening, avoid alcohol consumption, administer an insulin bolus, take certain medications, for example, in order to treat liver disease and to prevent the progression of liver disease.

Staging Liver Disease and/or Determining Development of Diabetes for a Patient with Liver Disease

If a patient is classified as a patient with liver disease at block 404, or if a healthy patient is determined to have liver disease at block 412, the method 400 continues to block 408. At block 408, therapy management engine 114 may monitor the patient's glucose levels using continuous analyte sensor 202. Continuous analyte data may, over time, monitor the patient for progression of liver disease and/or development of diabetes through the use of various metrics 130 to determine the patient's liver disease stage and/or presence of diabetes.

At block 414, therapy management engine 114 may provide feedback to the patient on the progression of the patient's liver disease and/or feedback on whether the patient is developing diabetes. Alternatively at block 414, therapy management engine 114 may provide feedback that the patient's liver disease is improving (e.g., regressing). The feedback provided to the patient may be based on glucose metrics (e.g., metrics 130) and other inputs. Similar to the feedback provided to a healthy patient at block 412, therapy management engine 114 may utilize a rules-based model or an ML model. Whether a rules-based model or an ML model is utilized, therapy management engine 114 may determine a liver disease stage and/or presence of diabetes based on the patient's historical or current glucose data and/or population based data relating to various glucose metrics, including, but not limited to, post-prandial glucose dynamics (e.g., the peak of the patient's post-prandial glucose spike, duration of hyperglycemia above baseline over a set time period post meal, rate of change of glucose as the patient's glucose returns to baseline), elevated baseline glucose level, nocturnal hypoglycemia, and dawn effect, as described herein.

Variations in a patient's glucose trace over time may be indicative of liver disease regression or progression. A patient's glucose trace may refer to a pattern of the patient's glucose levels over the course of a day, which may then be averaged over time compared to previous averages to identify variations in the patient's glucose levels. As discussed in more detail below, a patient's glucose trace may be related to a patient's post-prandial glucose dynamics, baseline glucose level, nocturnal hypoglycemia, dawn effect, etc. Glucose metrics discussed above in reference to a healthy patient at block 412 may also be relevant to determining whether a patient's liver disease is progressing.

Generally, a patient's post-prandial glucose dynamics may demonstrate an increase in glucose levels (to a post-prandial glucose spike maximum), followed by a decline in glucose levels back to baseline glucose levels within a certain time period following a meal. Therapy management engine 114 may determine an average for various post-prandial glucose dynamics of the patient during a first time period of monitoring the patient's glucose levels and compare with an average for various post-prandial glucose dynamics of the patient during a second time period following the first time period. For example, during the first time period (e.g., the first two weeks the patient's glucose levels are monitored) the patient may experience an average post-prandial glucose spike maximum of 150 mg/dL following a meal, and during a subsequent second time period, a patient may experience an average post-prandial glucose spike is 175 mg/dL. Therapy management engine 114 may determine the patient's average post-prandial glucose spike has increased over time (e.g., from 150 mg/dL to 175 mg/dL), which may be indicative of liver disease progression. Therapy management engine 114 may provide feedback to the patient that the patient's liver disease is progressing.

In certain embodiments, therapy management engine 114 determines a patient's baseline glucose level and/or the change in a patient's baseline glucose level over time, as discussed in reference to block 412 above. However, as liver disease progresses, a patient's baseline glucose level may continue to increase. In response to this increase, therapy management engine 114 may provide feedback to the patient indicating that the patient's liver disease is progressing.

Further, as discussed at block 412 in reference to healthy patients, patients with liver disease may have a greater post-prandial glucose area under the curve when compared with a healthy patient. Additionally, as a patient's liver disease becomes more severe, the post-prandial glucose area under the curve may increase. Therapy management engine 114 may monitor the patient's post-prandial glucose area under the curve and when the area under the curve is increasing over time, therapy management engine 114 may provide feedback to the patient that the patient's liver disease is progressing.

In certain embodiments, the amount of time it takes for the patient's glucose level to return to baseline indicates liver disease progression. For example, if a patient's average time to return to baseline glucose level following a meal is 2.5 hours, therapy management engine 114 may determine the patient has mild liver disease. At a future time, the patient may have an average time to return to baseline glucose level following a meal of 3 hours. Based on the increased time to return to baseline glucose level, therapy management engine 114 may determine the patient's liver disease is progressing.

In determining whether the patient's liver disease is progressing, therapy management engine 114 may also determine the type or content of the food the patient is consuming based on inputs 128, including food consumption information. The type or content of food that the patient consumes may affect the post-prandial glucose spike maximum, post-prandial glucose return to baseline, and/or the post-prandial glucose area under the curve of the patient. For example, if therapy management engine 114 determines the patient is consuming the same or similar foods over time and post-prandial glucose spike maximum continues to increase over time, therapy management engine 114 is able to accurately predict variability in the patient's post-prandial glucose spike is due to progression of liver disease, as opposed to variations in type or content of food. In another example, if therapy management engine 114 determines the patient is consuming a balanced meal including protein, carbohydrates, and fats, therapy management engine 114 may expect a slower return to baseline of glucose levels when compared with a patient consuming a glucose drink, for example.

In certain embodiments, therapy management engine 114 identifies variations in glucose metrics by controlled monitoring of glucose over set periods of time during the day, or when there are contextual similarities (e.g., when inputs 128 demonstrate that the patient is completing a specific type of exercise or consuming a specific type of food). For example, therapy management engine 114 may recommend a meal with a specific content (e.g., a meal that is mostly carbohydrates or a meal that is mostly glucose, for example) and therapy management engine 114 may monitor the patient's glucose response (e.g., post-prandial glucose spike). Over time, therapy management engine 114 may suggest the same food at a similar time each day to measure the patient's glucose response for comparison. If the patient's glucose response following the specific type of food increases over time (e.g., post-prandial glucose spike increases over time or the patient's time to return to baseline glucose levels following the meal increases), therapy management engine 114 may determine the patient's liver disease is progressing.

Alternatively, instead of being instructed to consume a specific food or specific content of food, therapy management engine 114 may instruct the patient to consume a glucose drink and therapy management engine 114 may monitor the patient's glucose response to the glucose drink. Therapy management engine 114 may instruct the patient to consume a glucose drink at a specific time of day once a week, for example, in order to monitor the patient's glucose response to the glucose drink, and therefore determine the patient's liver disease progression. Similar to the glucose monitoring following different types of food, therapy management engine 114 may determine the patient's liver disease is progressing if the patient's glucose response to the glucose drink increases over time. For example, if a patient's post-prandial glucose spike maximum is 175 mg/dL in response to a glucose drink on a first day, and at some time later (e.g., two weeks later), the patient's post-prandial glucose spike maximum is more than 200 mg/dL, therapy management engine 114 may provide feedback to the patient that the patient's liver disease is progressing.

Further, a patient's glucose response to a glucose drink may be used as a reference to determine how foods with different content (e.g., protein, fat, carbohydrate) alter a patient's glucose metrics, including post-prandial glucose spike maximum and time to return to glucose baseline, for example. The ratio of the change in glucose metrics and glucose traces between a glucose drink and foods with differing content may provide insight to therapy management engine 114 to determine the liver disease stage of the patient.

In certain embodiments, monitoring the patient's glucose response to exercise following a meal assists in determining whether the patient's liver disease is progressing and/or whether a specific exercise type is effective for preventing glucose spikes. For example, in response to receiving input that the patient consumed a meal comprising mostly carbohydrates, therapy management engine 114 may recommend the patient complete a light exercise session (e.g., a 15 minute walk) to prevent potential high glucose spikes and continue to monitor the patient's glucose response for the remainder of the day and throughout the night. If the patient's glucose levels remain within range throughout the day and the patient does not experience nocturnal hypoglycemia, therapy management engine 114 may recommend the same exercise in the future following carbohydrate heavy meals.

At block 414, therapy management engine 114 may simultaneously monitor a patient with liver disease for the development of diabetes using at least glucose levels from continuous analyte sensor 202. Therapy management engine 114 may determine a patient has diabetes through monitoring various glucose metrics, including glucose baseline level, post-prandial glucose area under the curve, dawn effect, and glucose minimum and maximum levels (e.g., glucose variability).

In certain embodiments, therapy management engine 114 determines a patient with liver disease has diabetes based on the patient's baseline glucose level. A diabetic patient, at rest, is less efficient at clearing glucose than a healthy patient or a patient with liver disease due to a diabetic patient's insulin resistance. Therefore, an elevation in baseline glucose level, either in between meal times or when the patient is at rest, may demonstrate that a patient has, or is developing, diabetes. For example, therapy management engine 114 may begin monitoring glucose levels of a patient with liver disease and therapy management engine 114 may determine the patient has an average baseline glucose level of 100 mg/dL. Then, at a later time period, therapy management engine 114 may determine a patient has an average baseline glucose level of 125 mg/dL. An increase in baseline glucose level may demonstrate that the patient is developing insulin resistance, and therefore, diabetes.

Further, therapy management engine 114 may determine a patient with liver disease is developing diabetes based on an increased area under the curve of post-prandial glucose spikes. Given a diabetic patient may suffer from insulin resistance, the ability for a patient with diabetes to metabolize glucose in the body following a meal may be slower than a healthy patient or a patient with liver disease. Therefore, following a meal and corresponding post-prandial glucose spike, the amount of time required for a diabetic patient's glucose to return to baseline glucose levels may be longer and the area under the curve of the post-prandial glucose spike may be more than a healthy patient or a patient with liver disease. For example, therapy management engine 114 may determine a patient's average post-prandial glucose spike area under the curve increases over time (e.g., over two months, for example). Then, based on the increase in post-prandial glucose spike area under the curve, therapy management engine 114 may provide feedback to the patient that the patient is developing diabetes, or has diabetes.

Further, therapy management engine 114 may determine a patient with liver disease has, or is developing a dawn effect over time. Diabetic patients are known to suffer from a dawn effect, where, due to circadian rhythm cycles, glucose values elevate slightly in the morning as the body wakes from sleep. Therefore, as the patient begins to develop diabetes, the patient may begin to experience a dawn effect, and/or the dawn effect may become more pronounced.

Based on the determination of disease state at block 414, at block 420, therapy management engine 114 may provide recommendations for treatment to improve the patient's liver disease stage and/or prevent further development of diabetes. If therapy management engine 114 determines a patient's liver disease is progressing and/or the patient is developing diabetes at block 414, therapy management engine 114 may instruct the patient to alter meal times (e.g., eat a light meal before bed), complete an exercise session after large meals, avoid exercising in the evening, avoid alcohol consumption, take certain medications, etc. to prevent further progression of liver disease and/or prevent further development of diabetes.

For example, therapy management engine 114 may determine statin medications (e.g., medications to manage LDL cholesterol levels) prescribed to the patient via patient inputs and monitor corresponding glucose data to identify improvement or deterioration of liver disease. Monitoring glucose data while a patient is on statin medications may be beneficial as certain statins may increase glucose levels, depending on the individual patient and the type of statin. Therapy management engine 114 may determine when the medication is improving liver disease symptoms and not negatively effecting glucose levels. If the medication is improving liver disease symptoms and glucose levels, therapy management engine 114 may suggest the patient continue taking the medication. Alternatively, if the patient's liver disease is progressing and/or glucose levels are worsening (e.g., baseline glucose levels are increasing, the patient is experiencing nocturnal hypoglycemia, and/or the post-prandial glucose spike maximum is increasing), therapy management engine 114 may suggest the patient switch medications or add medications based on the patient's disease determination (e.g., whether the patient's liver disease is progressing and/or the patient is developing diabetes). For example, therapy management engine 114 may recommend a second liver-specific medication and/or therapy if the patient's liver disease is progressing and/or a glucose-specific medication and/or therapy if the patient is developing diabetes.

Staging Liver Disease and/or Determining Liver Disease Development for a Diabetic Patient

If a patient is classified as a diabetic patient at block 404, the method continues to block 410. At block 410, therapy management engine 114 may monitor or continue monitoring the diabetic patient's glucose levels using continuous analyte sensor 202. Continuous analyte data (e.g., continuous glucose data) may, over time, provide a determination that a diabetic patient also has liver disease, and/or provide monitoring for a diabetic patient with known liver disease to determine worsening or improvement of liver disease.

At block 416, therapy management engine 114 may provide feedback to the diabetic patient on the presence of liver disease and/or the development of liver disease based on glucose metrics of the patient. As discussed above at block 412 and block 414 in reference to healthy patients and patients with liver disease, various glucose metrics may be monitored to determine when a diabetic patient may have liver disease and when a patient's liver disease is progressing, as well as when a patient may have diabetes. While the above referenced metrics are applicable to liver disease diagnosis and staging in a diabetic patient, some glucose metrics and trends in glucose traces may be unique to a patient with liver disease who also has, or later develops, diabetes.

For example, post-prandial glucose spikes may be present in a diabetic patient without liver disease, however, the post-prandial glucose spike magnitude may be less than that of a patient with liver disease alone, or a patient with diabetes and liver disease. For example, therapy management engine 114 may be trained with data that classifies a patient as a patient with liver disease or a patient with liver disease and diabetes if the patient has an average post-prandial glucose spike magnitude of 200 mg/dL. Alternatively, therapy management engine 114 may be trained to classify a patient as a diabetic patient without liver disease if the patient has an average post-prandial glucose spike magnitude of 160 mg/dL. Therefore, as therapy management engine 114 monitors a patient with diabetes, and if a patient's average post-prandial glucose spike magnitude increases over time (e.g., from 160 mg/dL to 200 mg/dL), therapy management engine 114 may provide feedback to the patient that the patient has liver disease or is developing liver disease.

Additionally, therapy management engine 114 may identify that a diabetic patient is experiencing nocturnal hypoglycemia based on glucose metrics of a diabetic patient. Therapy management engine 114 may determine a patient has nocturnal hypoglycemia if a patient's glucose levels begin to decrease at night relative to the patient's historical nighttime glucose levels and/or relative to the patient's daytime baseline glucose levels (e.g., more than 30 mg/dL below daytime baseline glucose levels, or 10 mg/dL below historical nighttime glucose levels, for example). Based on the identification of nocturnal hypoglycemia, therapy management engine 114 may determine the patient has liver disease or is developing liver disease. For example, if a patient has a daytime baseline glucose level of 120 mg/dL and a nighttime glucose level of 125 mg/dL, but at a later time point has a daytime baseline glucose level of 120 mg/dL and a nighttime glucose level of 90 mg/dL, therapy management engine 114 may provide feedback to the patient that the patient has liver disease or is developing liver disease.

Therapy management engine 114 may also monitor glucose variability (e.g., glucose minimum and glucose maximum levels) to determine when a patient with diabetes may be developing liver disease. Glucose variability may be measured while the patient is at rest, or while the patient is consuming a specific type of food or drink. For example, a patient with liver disease may demonstrate increased glucose variability to specific foods, including alcohol, when compared with a diabetic patient without liver disease. Patients with liver disease may not be able to maintain normal glucose levels in response to consuming alcohol. The inability for patient's with liver disease to metabolize alcohol is based on the inability for the liver to suppress glucose production as the body begins to metabolize alcohol instead glucose. Therefore, if a diabetic patient develops increased glucose variability over time, especially in response to consuming alcohol, then therapy management engine 114 may provide feedback to the diabetic patient that the patient has, or is developing liver disease.

At block 422, based on the determination of development of liver disease at block 416 and the patient's glucose metrics, therapy management engine 114 may provide recommendations for treatment to improve the patient's liver disease stage and/or risk of developing liver disease. If therapy management engine 114 determines the patient has liver disease or is developing liver disease at block 416, therapy management engine 114 may instruct the patient to alter meal times, avoid alcohol consumption, avoid exercising in the evening, avoid certain medications, etc. to prevent further progression of liver disease or further development of liver disease.

In certain embodiments, therapy management engine 114 instructs the patient to take certain common diabetic medications when the diabetic patient has or is developing liver disease. For example, therapy management engine 114 may suggest the diabetic patient with liver disease take a GLP-1, Thiazolidinedione (TZD), or Metformin, which still may be effective for a diabetic patient with liver disease. Certain other common diabetic medications may not be effective for a patient with diabetes and liver disease.

Additionally, therapy management engine 114 may recommend some exercise (e.g., Zone 2 aerobic exercise) to reduce glucose levels, particularly after a meal or high sugar food, as described herein. However, if the diabetic patient is determined to have liver disease or is at risk of developing liver disease, therapy management engine 114 may instruct the patient to not exercise in the evening or nighttime (e.g., not to exercise after 3 P.M., for example) to avoid nocturnal hypoglycemia as the body continues to metabolize glucose after an exercise session.

Further, therapy management engine 114 may monitor comorbidities and medications of the patient, as provided via inputs 128, as certain comorbidities may affect glucose metrics. For example, if a diabetic patient has hypertension and is on hypertensive medications, the hypertensive medications may affect baseline glucose level, glucose rates of change, post-prandial glucose spike maximum, etc. By monitoring comorbidities and medications the patient may be taking, therapy management engine 114 may correct for the affect the medications have on glucose levels. Therefore, therapy management engine 114 may more accurately determine the glucose level increases related to liver health in order to diagnose or determine a risk of developing liver disease.

Additionally, medication history, including changes in medications, may be utilized by therapy management engine 114 to determine when glucose metrics and/or glucose trace changes are related to medication. For example, if the patient provides an input suggesting that the patient is starting a new medication, therapy management engine 114 may determine increased glucose levels and glucose spikes over the next time period to be indicative of the patient's body reacting to the change in medication. Therapy management engine 114 may discard glucose data related to a known medication change and resume monitoring for development of liver disease after some time period (e.g., 2 weeks) to allow the patient's body time to adjust to the new medication.

In certain embodiments, a patient with diabetes and liver disease wants to lose weight. To accomplish this goal, therapy management engine 114 may suggest specific exercise and meal recommendations in order for the patient to lose weight while maintaining glucose levels in range, specifically at night. For example, therapy management engine 114 may recommend the patient exercise early in the day, and then suggest the patient eat macronutrient balanced meals throughout the day with complex carbohydrates at dinner to rebuild glycogen stores prior to nighttime and avoid nocturnal hypoglycemia. However, therapy management engine 114 may recommend the patient avoids eating a meal or foods that are high in fat. If the patient chooses to exercise later in the day (e.g., in the afternoon), therapy management engine 114 may suggest a meal that is high in carbohydrates before bed to avoid nocturnal hypoglycemia. However, therapy management engine 114 may recommend the patient exercise early in the day in the future, as a high carbohydrate meal in the afternoon or evening may hinder weight loss over time.

In certain embodiments, a patient is following a ketogenic diet to lose weight, treat liver disease, and/or prevent progression of liver disease. The ketogenic diet may only be effective if the patient appropriately enters ketosis, which requires a specific ratio of fat to protein or carbohydrate consumption. Continuously monitoring glucose levels, especially when correlated with ketone levels, may assist a patient in determining whether they are in ketosis, therefore improving the effectiveness of a ketogenic diet for liver disease treatment and/or weight loss.

In certain embodiments, diabetic patients are at risk for developing kidney disease while therapy management engine 114 is monitoring the patient for development of liver disease. In order for therapy management engine 114 to distinguish between liver disease and kidney disease, therapy management engine 114 may monitor for decreasing baseline glucose levels, increasing glucose level fluctuations, increased glucose level variability, a decrease in overnight minimum glucose levels (e.g., nocturnal hypoglycemia), an increase in a post-prandial glucose spike maximum, a shorter period of time required for a patient's glucose level to return to baseline level, and an increase in a rate of change of decline of glucose levels following a meal which may be indicative of worsening kidney function and/or development of kidney disease. In addition to glucose focused metrics, other analyte levels and analyte metrics may be utilized for determining when a patient is developing kidney disease, including but not limited to, lactate, creatinine, cystatin C, proteinuria, albumin creatinine ratio, etc.

For example, baseline glucose levels may demonstrate a patient has kidney disease, as opposed to or in addition to liver disease, if the patient's baseline glucose decreases and/or if the patient's baseline glucose is lower than a historical patient population with liver disease. A patient's baseline glucose may change based on the patient's health, specifically in response to a decline in kidney and/or liver health. In another example, therapy management engine 114 may determine a patient has kidney disease or is experiencing worsening kidney function if the patient's glucose level fluctuations increase and the patient's glucose levels demonstrate higher rates of change. In another example, a decrease in overnight minimum glucose levels (e.g., when the patient experiences low glucose over time while sleeping (e.g., after several hours of being asleep)) may be indicative of kidney disease. Generally, both patients suffering from liver disease and patients suffering from kidney disease may experience normal to high glucose levels upon falling asleep, however, patients with kidney disease may experience lower glucose levels after a few hours of sleep.

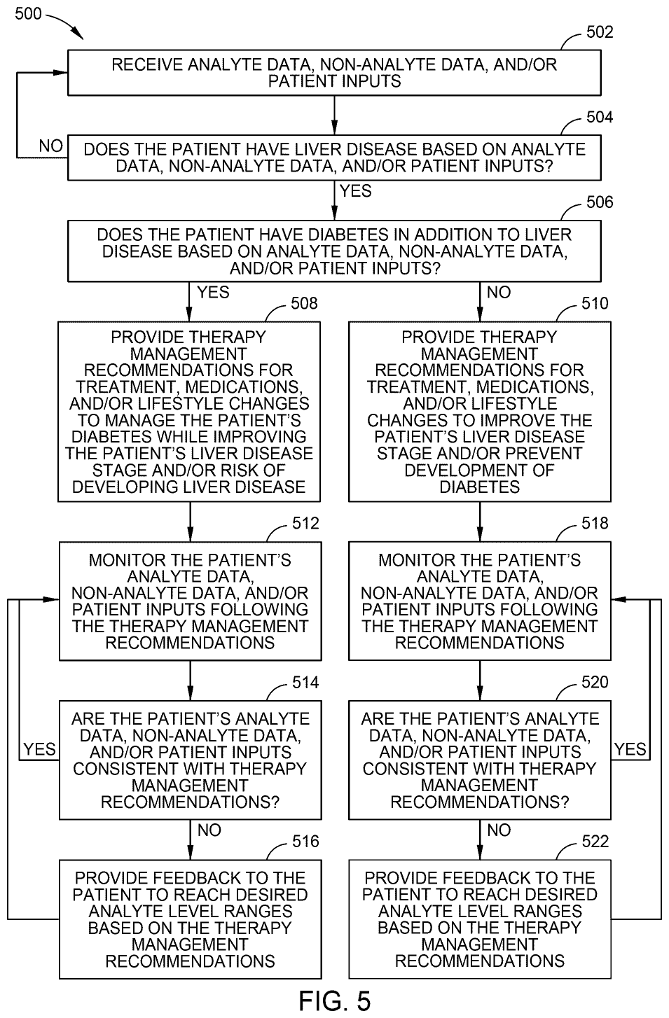

FIG. 5 describes an example method 500 for monitoring progression of liver disease and/or diabetes based on one or more therapy management recommendations as described in reference to FIG. 4. The therapy management recommendations provided by therapy management engine 114 may be modified to prevent progression of liver disease and/or diabetes or to continue to improve a patient's analyte and/or non-analyte data over time. At block 502, method 500 begins by therapy management engine 114 receiving analyte data from continuous analyte monitoring system 104, and continuous analyte sensor(s) 202 illustrated in FIG. 2. For example, therapy management engine 114 may receive glucose data. Further, therapy management engine 114 may receive data from patient inputs. The patient inputs may be received in a variety of ways. For example, the inputs may be received or retrieved from the patient profile 118, which includes demographic info 120, disease info 122, and medication info 124, inputs 128, metrics 130, etc. Inputs may also be received as patient input through the patient interface of a display device 107. Even further, therapy management engine 114 may receive non-analyte data from one or more non-analyte sensors 206.

At block 504, therapy management engine 114 determines whether the patient has liver disease based on the analyte data, non-analyte data, and/or patient inputs received at block 502. Therapy management engine 114 may utilize a rules-based model or a ML model as described in reference to FIG. 4 to determine whether the patient has liver disease. If the patient does not have liver disease, therapy management engine 114 returns to block 402 to continue to receive the patient's analyte data, non-analyte data, and patient inputs over time. Alternatively, if the patient is determined to have liver disease, therapy management engine 114 proceeds to block 506.

At block 506, therapy management engine 114 determines whether the patient has diabetes in addition to liver disease based on analyte data, non-analyte data, and/or patient inputs. Therapy management engine 114 may utilize a rules-based model or an ML model as described in reference to FIG. 4 to determine whether the patient has diabetes. If the patient has diabetes, therapy management engine 114 may proceed to block 508. At block 508, therapy management engine 114 may provide therapy management recommendations for treatment to manage the patient's diabetes while improving the patient's liver disease stage and/or risk of developing liver disease. The therapy management recommendations provided to the patient at block 508 may be similar to those described in reference to block 422 of FIG. 4.

Following block 508, therapy management engine 114 proceeds to block 512. At block 512, therapy management engine 114 continues to monitor the patient's analyte data, non-analyte data, and/or patient inputs following the therapy management recommendations. At block 514, therapy management engine 114 determines whether the patient's analyte data, non-analyte data, and/or patient inputs are consistent with historical patient data and/or expected patient data based on patients following similar therapy management recommendations. Therapy management engine 114 may determine whether the patient's analyte data, non-analyte data, and/or patient inputs are consistent with therapy management recommendations to confirm whether the patient is compliant with the recommendations and/or to determine whether the recommendations are appropriate for the specific patient.

If the patient's analyte data, non-analyte data, and/or patient inputs are consistent with the patient's therapy management recommendations, therapy management engine 114 may proceed to block 512 to continue to monitor the patient's analyte data, non-analyte data, and/or patient inputs to confirm the patient is following the therapy management recommendations. If the patient's analyte data, non-analyte data, and/or patient inputs are not consistent with the patient's therapy management recommendations, therapy management engine 114 proceeds to block 516. At block 516, therapy management engine 114 may provide feedback to the patient to assist the patient in reaching the desired analyte data ranges based on the therapy management recommendations. In certain embodiments, the feedback to the patient includes different and/or modified therapy management recommendations.

Following block 516, therapy management engine 114 may return to block 512 to continue to monitor the patient's analyte data, non-analyte data, and/or patient inputs following the different and/or updated therapy management recommendations.

Alternatively, from block 506, therapy management engine 114 may determine the patient does not have diabetes in addition to liver disease and proceed to block 510. At block 510, therapy management engine 114 may provide therapy management recommendations for treatment to improve the patient's liver disease stage and/or prevent the development of diabetes. The therapy management recommendations provided to the patient at block 510 may be similar to those described in reference to block 420 of FIG. 4.

Following block 510, therapy management engine 114 proceeds to block 518. At block 518, therapy management engine 114 continues to monitor the patient's analyte data, non-analyte data, and/or patient inputs following the therapy management recommendations. At block 520, therapy management engine 114 determines whether the patient's analyte data, non-analyte data, and/or patient inputs are consistent with historical patient data and/or expected patient data following similar therapy management recommendations. Therapy management engine 114 may determine whether the patient's analyte data, non-analyte data, and/or patient inputs are consistent with therapy management recommendations to confirm whether the patient is compliant with the recommendations and/or to determine whether the recommendations are appropriate for the specific patient.

If the patient's analyte data, non-analyte data, and/or patient inputs are consistent with the patient's therapy management recommendations, therapy management engine 114 may return to block 518 to continue to monitor the patient's analyte data, non-analyte data, and/or patient inputs to confirm the patient is following the therapy management recommendations. If the patient's analyte data, non-analyte data, and/or patient inputs are not consistent with the patient's therapy management recommendations, therapy management engine 114 may proceed to block 522. At block 522, therapy management engine 114 provides feedback to the patient to assist the patient in reaching the desired analyte data ranges based on the therapy management recommendations. In certain embodiments, the feedback to the patient includes different and/or modified therapy management recommendations.

Following block 522, therapy management engine 114 may return to block 518 to continue to monitor the patient's analyte data, non-analyte data, and/or patient inputs following the different and/or updated therapy management recommendations.

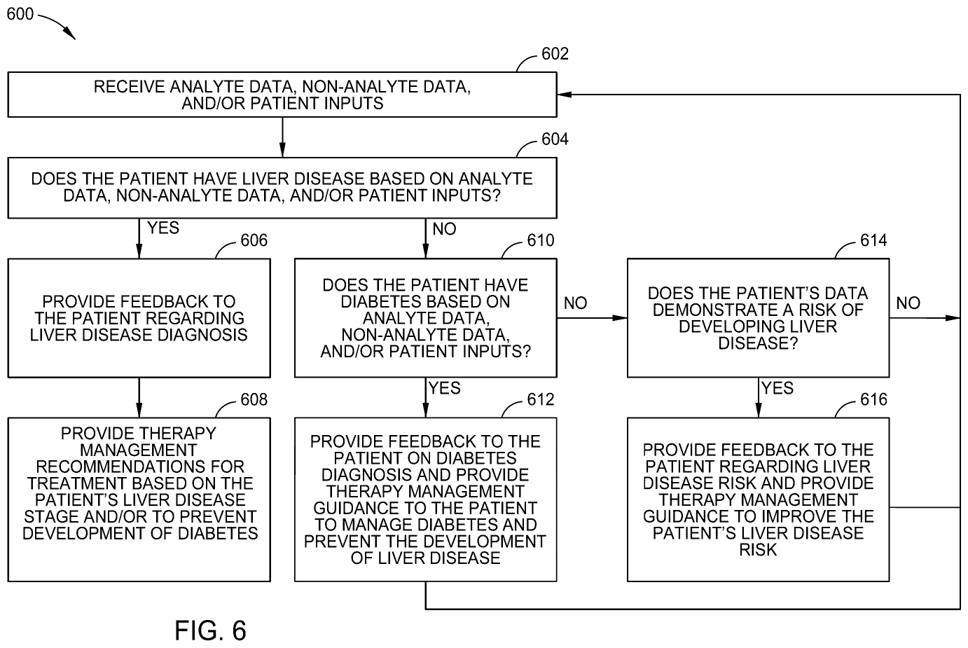

FIG. 6 describes an example method 600 for determining a patient's liver disease risk and providing therapy management recommendations to reduce the patient's liver disease risk and/or prevent liver disease development. At block 602, method 600 begins by therapy management engine 114 receiving analyte data from continuous analyte monitoring system 104, and continuous analyte sensor(s) 202 illustrated in FIG. 2. For example, therapy management engine 114 may receive glucose data. Further, therapy management engine 114 may receive data from patient inputs. The patient inputs may be received in a variety of ways. For example, the inputs may be received or retrieved from the patient profile 118, which includes demographic info 120, disease info 122, and medication info 124, inputs 128, metrics 130, etc. Inputs may also be received as patient input through the patient interface of a display device 107. Even further, therapy management engine 114 may receive non-analyte data from one or more non-analyte sensors 206.

Following block 602, therapy management proceeds to block 604. At block 604, therapy management engine 114 determines whether the patient has liver disease based on analyte data, non-analyte data, and/or patient inputs. Therapy management engine 114 may utilize a rules-based model or an ML model as described in reference to FIG. 4 to determine whether the patient has liver disease. If the patient has liver disease, therapy management engine 114 may proceed to block 606. At block 606, therapy management engine 114 may provide feedback to the patient regarding the patient's liver disease diagnosis. At block 608, therapy management engine 114 may further provide therapy management recommendations to the patient for treatment based on the patient's liver disease stage and/or to prevent the development of diabetes. The therapy management recommendations may be consistent with the therapy management recommendations as described in reference to blocks 420 and 422 of FIG. 4.

Returning to block 602, if therapy management engine 114 determines the patient does not have liver disease based on analyte data, non-analyte data, and/or patient inputs, therapy management engine 114 may proceed to block 610. At block 610, therapy management engine 114 may determine whether the patient has diabetes based on the patient's analyte data, non-analyte data, and/or patient inputs. Therapy management engine 114 may utilize a rules-based model or an ML model as described in reference to FIG. 4 to determine whether the patient has diabetes. If therapy management engine 114 determines the patient has diabetes, therapy management engine 114 may proceed to block 612. At block 612, therapy management engine 114 provides feedback to the patient on the patient's diabetes diagnosis and provides therapy management recommendations to the patient to manage diabetes and prevent the development of liver disease. The therapy management recommendations may be similar to those described in reference to block 422 of FIG. 4. Following block 612, therapy management engine 114 may return to block 602 to continue monitoring the patient's analyte data, non-analyte data, and/or patient inputs to monitor the patient's diabetes and/or development of liver disease.

Returning to block 610, if therapy management engine determines that the patient does not have diabetes, therapy management engine 114 proceeds to block 614. At block 614, therapy management engine 114 may determine whether the patient's analyte data, non-analyte data, and/or patient inputs demonstrate an increased risk of developing liver disease. If the patient's data demonstrates an increased risk of developing liver disease, therapy management engine 114 may proceed to block 616. At block 616, therapy management engine 114 may provide feedback to the patient regarding the patient's increased liver disease risk and provide therapy management recommendations to the patient to improve or eliminate the patient's liver disease risk. Following block 616, therapy management engine 114 may return to block 602 to continue monitoring the patient's analyte data, non-analyte data, and/or patient inputs over time. Alternatively, if the patient's data does not demonstrate an increased risk of developing liver disease, therapy management engine 114 may return to block 602 to continue monitoring the patient's analyte data, non-analyte data, and/or patient inputs over time.

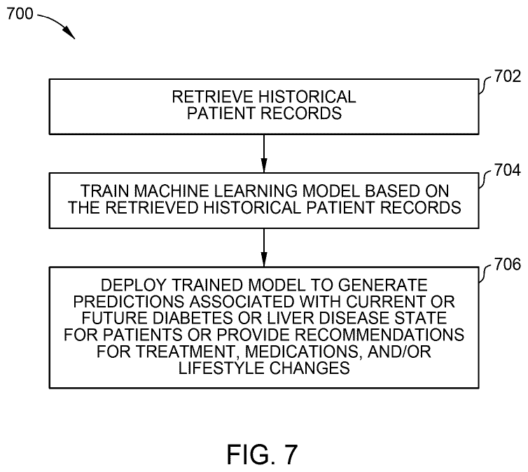

In certain embodiments, machine learning models deployed by therapy management engine 114 include one or more models trained by training server system 140, as illustrated in FIG. 1. FIG. 7 describes in further detail techniques for training the machine learning model(s) deployed by therapy management engine 114 for classifying a patient, predicting a current or future diabetes or liver disease state, and/or providing therapy management recommendations for treatment.

FIG. 7 is a flow diagram depicting a method 700 for training machine learning models to classify a patient, predict a patient's current or future diabetes or liver disease state, and/or provide therapy management recommendations to a patient based on disease state. In certain embodiments, the method 700 is used to train models for predicting a current or future diabetes or liver disease state, as illustrated in FIG. 1.

Method 700 begins, at block 702, by training server system, such as training server system 140 illustrated in FIG. 1, retrieving data from historical records database, such as historical records database 112 illustrated in FIG. 1. As mentioned herein, historical records database 112 may provide a repository of up-to-date information and historical information for patients of a continuous analyte monitoring system and connected mobile health application, such as patients of continuous analyte monitoring system 104 and application 106 illustrated in FIG. 1, as well as data for one or more patients who are not, or were not previously, patients of continuous analyte monitoring system 104 and/or application 106. In certain embodiments, historical records database 112 includes one or more data sets of historical patients who are healthy patients, patients with liver disease, and diabetic patients.

Retrieval of data from historical records database 112 by training server system 140, at block 702, may include the retrieval of all, or any subset of, information maintained by historical records database 112. For example, where historical records database 112 stores information for 100,000 patients (e.g., non-patients and patients of continuous analyte monitoring system 104 and application 106), data retrieved by training server system 140 to train one or more machine learning models may include information for all 100,000 patients or only a subset of the data for those patients, e.g., data associated with only 50,000 patients or only data from the last ten years.

As an illustrative example, integrating with on premises or cloud based medical record databases through Fast Healthcare Interoperability Resources (FHIR), web application programming interfaces (APIs), Health Level 7 (HL7), and or other computer interface language may enable aggregation of healthcare historical records for baseline assessment in addition to the aggregation of de-identifiable patient data from a cloud based repository. Similarly, when integrating into the medical record databases, the integration may be accomplished by directly interfacing with the electronic medical record system or through one or more intermediary systems (e.g., an interface engine, etc.).

As an illustrative example, at block 702, training server system 140 may retrieve information for 100,000 patients with various classifications (e.g., healthy patient, patient with liver disease, and/or diabetic patient) stored in historical records database 112 to train a model to predict a current or future diabetes or liver disease state of a patient and provide therapy management recommendations to the patient. Each of the 100,000 patients may have a corresponding data record (e.g., based on their corresponding patient profile)), stored in historical records database 112. Each patient profile 118 may include information, such as information discussed with respect to FIG. 3.

The training server system 140 then uses information in each of the records to train an artificial intelligence or ML model (for simplicity referred to as “ML model” herein). Examples of types of information included in a patient's profile were provided above. The information in each of these records may be featurized (e.g., manually or by training server system 140), resulting in features that can be used as input features for training the ML model. For example, a patient record may include or be used to generate features related to the patient's demographic information (e.g., an age of a patient, a gender of the patient, etc.), analyte information, such as glucose metrics (e.g., post-prandial glucose spike, post-prandial glucose area under the curve, nocturnal hypoglycemia, glucose baseline level, other glucose metrics described herein), non-analyte information, and/or any other data points in the patient record (e.g., inputs 128, metrics 130, etc.). Features used to train the machine learning model(s) may vary in different embodiments.

In certain embodiments, each historical patient record retrieved from historical records database 112 is further associated with a label indicating a patient classification, (e.g., a healthy patient, a patient with liver disease, and/or a patient with diabetes), current diabetes or liver disease state, etc. What the record is labeled with would depend on what the model is being trained to predict.

At block 704, method 700 continues by training server system 140 training one or more machine learning models based on the features and labels associated with the historical patient records. In some embodiments, the training server does so by providing the features as input into a model. This model may be a new model initialized with random weights and parameters, or may be partially or fully pre-trained (e.g., based on prior training rounds). Based on the input features, the model-in-training generates some output. In certain embodiments, the output includes classification of the patient, a current or future diabetes or liver disease state, and/or therapy management recommendations for treatment to improve the patient's diabetes or liver disease state, or similar outputs. Note that the output could be in the form of a classification, a therapy management recommendation, and/or other types of output.

In certain embodiments, training server system 140 compares this generated output with the actual label associated with the corresponding historical patient record to compute a loss based on the difference between the actual result and the generated result. This loss is then used to refine one or more internal weights and parameters of the model (e.g., via backpropagation) such that the model learns to predict a current or future diabetes or liver disease state, and/or provide therapy management recommendations for treatment to improve the patient's diabetes or liver disease state more accurately.

One of a variety of machine learning algorithms may be used for training the model(s) described above. For example, one of a supervised learning algorithm, a neural network algorithm, a deep neural network algorithm, a deep learning algorithm, etc. may be used.

At block 706, training server system 140 deploys the trained model(s) to make predictions associated with current or future diabetes or liver disease state during runtime. In some embodiments, this includes transmitting some indication of the trained model(s) (e.g., a weights vector) that can be used to instantiate the model(s) on another device. For example, training server system 140 may transmit the weights of the trained model(s) to therapy management engine 114, which could execute on display device 107, etc. The model(s) can then be used to determine, in real-time, a current or future diabetes or liver disease state of a patient using application 106, and/or make other types of therapy management recommendations discussed above. In certain embodiments, the training server system 140 continues to train the model(s) in an “online” manner by using input features and labels associated with new patient records.

Further, similar methods for training illustrated in FIG. 7 using historical patient records may also be used to train models using patient-specific records to create more personalized models for making predictions associated with patient classification, and/or current or future diabetes or liver disease state. For example, a model trained using historical patient records that is deployed for a particular patient, may be further re-trained after deployment. For example, the model may be re-trained after the model is deployed for a specific patient to create a more personalized model for the patient. The more personalized model may be able to more accurately make predictions on disease state of the patient based on the patient's own data (as opposed to only historical patient record data), including the patient's own inputs 128 and metrics 130.

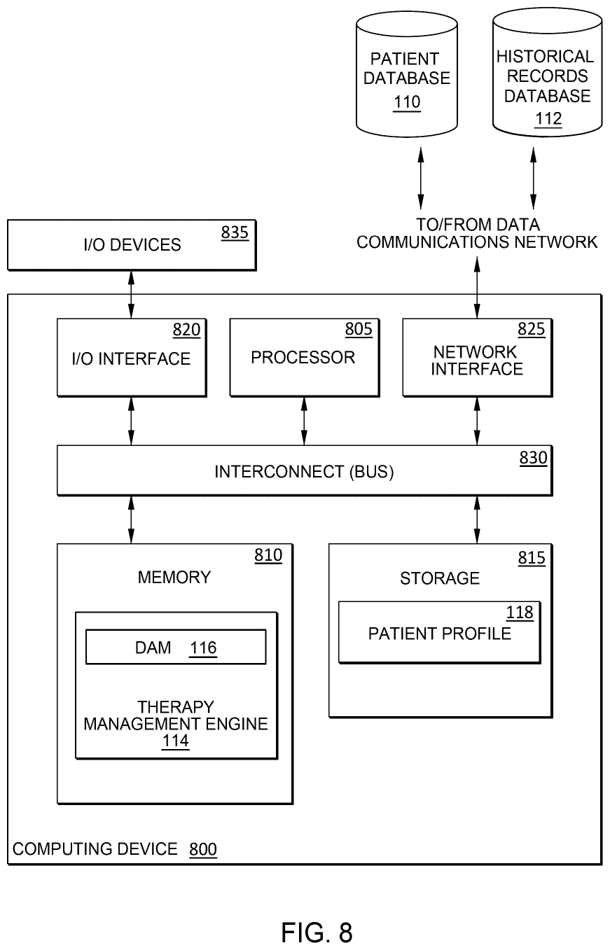

FIG. 8 is a block diagram depicting a computing device 800 configured to execute a therapy management engine (e.g., therapy management engine 114), according to certain embodiments disclosed herein. Although depicted as a single physical device, in embodiments, computing device 800 may be implemented using virtual device(s), and/or across a number of devices, such as in a cloud environment. As illustrated, computing device 800 includes a processor 805, memory 810, storage 815, a network interface 825, and one or more I/O interfaces 820. In the illustrated embodiment, processor 805 retrieves and executes programming instructions stored in memory 810, as well as stores and retrieves application data residing in storage 815. Processor 805 is generally representative of a single CPU and/or GPU, multiple CPUs and/or GPUs, a single CPU and/or GPU having multiple processing cores, and the like. Memory 810 is generally included to be representative of a random-access memory. Storage 815 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, input and output (I/O) devices 835 (such as keyboards, monitors, etc.) can be connected via the I/O interface(s) 820. Further, via network interface 825, computing device 800 can be communicatively coupled with one or more other devices and components, such as patient database 110. In certain embodiments, computing device 800 is communicatively coupled with other devices via a network, which may include the Internet, local network(s), and the like. The network may include wired connections, wireless connections, or a combination of wired and wireless connections. As illustrated, processor 805, memory 810, storage 815, network interface(s) 825, and I/O interface(s) 820 are communicatively coupled by one or more interconnects 830. In certain embodiments, computing device 800 is representative of display device 107 associated with the patient. In certain embodiments, as discussed above, the display device 107 can include the patient's laptop, computer, smartphone, and the like. In another embodiment, computing device 800 is a server executing in a cloud environment.

In the illustrated embodiment, storage 815 includes patient profile 118. Memory 810 includes therapy management engine 114, which itself includes DAM 116.

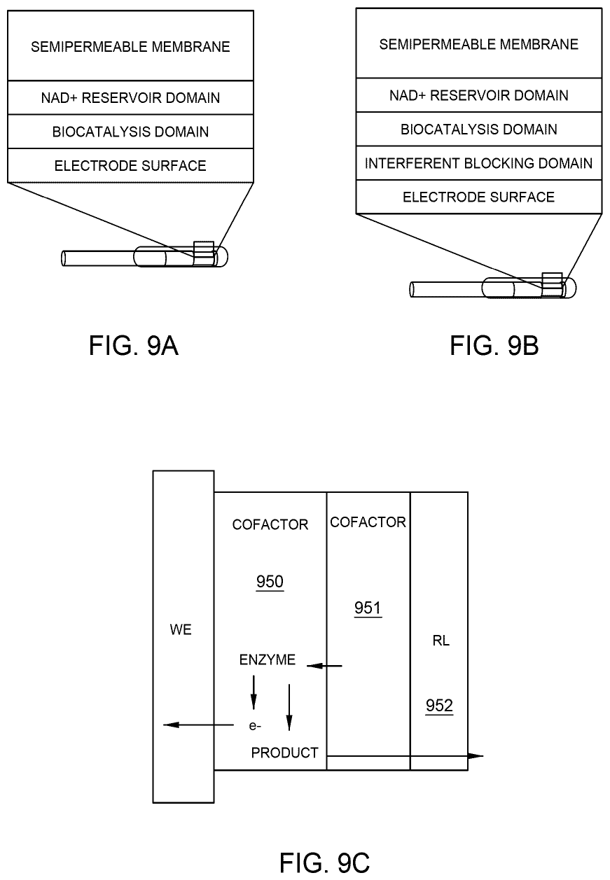

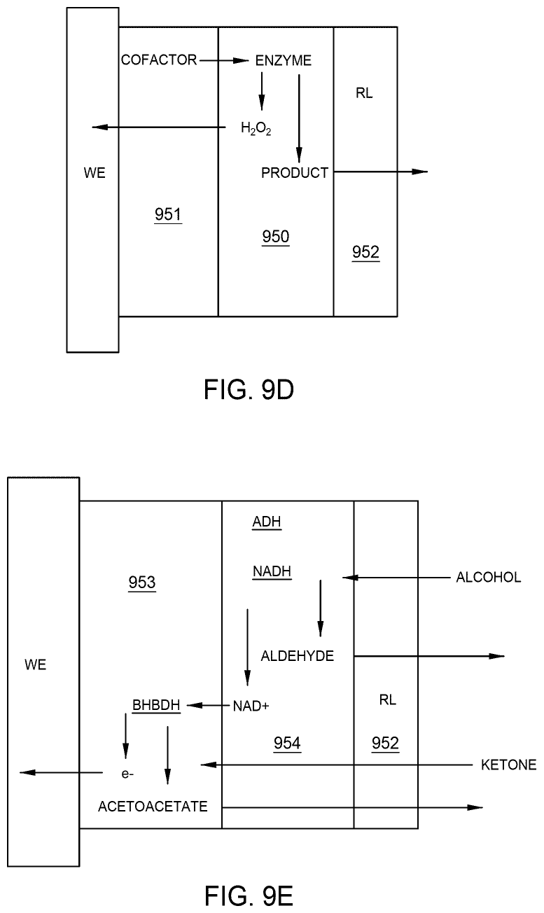

As described above, continuous analyte monitoring system 104, described in relation to FIG. 1, may be a multi-analyte sensor system including a multi-analyte sensor. FIG. 9A-13 describe example multi-analyte sensors used to measure multiple analytes.

The phrases “analyte-measuring device,” “analyte-monitoring device,” “analyte-sensing device,” and/or “multi-analyte sensor device” as used herein are broad phrases, and are to be given their ordinary and customary meaning to a person of ordinary skill in the art (and are not to be limited to a special or customized meaning), and refer without limitation to an apparatus and/or system responsible for the detection of, or transduction of a signal associated with, a particular analyte or combination of analytes. For example, these phrases may refer without limitation to an instrument responsible for detection of a particular analyte or combination of analytes. In one example, the instrument includes a sensor coupled to circuitry disposed within a housing, and configure to process signals associated with analyte concentrations into information. In one example, such apparatuses and/or systems are capable of providing specific quantitative, semi-quantitative, qualitative, and/or semi qualitative analytical information using a biological recognition element combined with a transducing (detecting) element.

The terms “biosensor” and/or “sensor” as used herein are broad terms and are to be given their ordinary and customary meaning to a person of ordinary skill in the art (and are not to be limited to a special or customized meaning), and refer without limitation to a part of an analyte measuring device, analyte-monitoring device, analyte sensing device, and/or multi-analyte sensor device responsible for the detection of, or transduction of a signal associated with, a particular analyte or combination of analytes. In one example, the biosensor or sensor generally comprises a body, a working electrode, a reference electrode, and/or a counter electrode coupled to body and forming surfaces configured to provide signals during electrochemically reactions. One or more membranes can be affixed to the body and cover electrochemically reactive surfaces. In one example, such biosensors and/or sensors are capable of providing specific quantitative, semi-quantitative, qualitative, semi qualitative analytical signals using a biological recognition element combined with a transducing (detecting) element.

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In one example, the sensing portion, sensing membrane, and/or sensing mechanism generally comprise an electrode configured to provide signals during electrochemically reactions with one or more membranes covering electrochemically reactive surface. In one example, such sensing portions, sensing membranes, and/or sensing mechanisms can provide specific quantitative, semi-quantitative, qualitative, semi qualitative analytical signals using a biological recognition element combined with a transducing (detecting) element.

The phrases “biointerface membrane” and “biointerface layer” as used interchangeably herein are broad phrases, and are to be given their ordinary and customary meaning to a person of ordinary skill in the art (and are not to be limited to a special or customized meaning), and refer without limitation to a permeable membrane (which can include multiple domains) or layer that functions as a bioprotective interface between patient tissue and an implantable device. The terms “biointerface” and “bioprotective” are used interchangeably herein.

The term “cofactor” as used herein is a broad term, and is to be given its ordinary and customary meaning to a person of ordinary skill in the art (and is not to be limited to a special or customized meaning), and refers without limitation to one or more substances whose presence contributes to or is required for analyte-related activity of an enzyme. Analyte-related activity can include, but is not limited to, any one of or a combination of binding, electron transfer, and chemical transformation. Cofactors are inclusive of coenzymes, non-protein chemical compounds, metal ions and/or metal organic complexes. Coenzymes are inclusive of prosthetic groups and co-substrates.

The term “continuous” as used herein is a broad term, and is to be given its ordinary and customary meaning to a person of ordinary skill in the art (and is not to be limited to a special or customized meaning), and refers without limitation to an uninterrupted or unbroken portion, domain, coating, or layer.

The phrases “continuous analyte sensing” and “continuous multi-analyte sensing” as used herein are broad phrases, and are to be given their ordinary and customary meaning to a person of ordinary skill in the art (and is not to be limited to a special or customized meaning), and refers without limitation to the period in which monitoring of analyte concentration is continuously, continually, and/or intermittently (but regularly) performed, for example, from about every second or less to about one week or more. In further examples, monitoring of analyte concentration is performed from about every 2, 3, 5, 7, 10, 15, 20, 25, 30, 35, 40, 45, 50, 55, or 60 seconds to about every 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50 or 9.75 minutes. In further examples, monitoring of analyte concentration is performed from about 10, 20, 30, 40 or 50 minutes to about every 1, 2, 3, 4, 5, 6, 7 or 8 hours. In further examples, monitoring of analyte concentration is performed from about every 8 hours to about every 12, 16, 20, or 24 hours. In further examples, monitoring of analyte concentration is performed from about every day to about every 1.5, 2, 3, 4, 5, 6, or 7 days. In further examples, monitoring of analyte concentration is performed from about every week to about every 1.5, 2, 3 or more weeks.

The term “coaxial” as used herein is to be construed broadly to include sensor architectures having elements aligned along a shared axis around a core that can be configured to have a circular, elliptical, triangular, polygonal, or other cross-section such elements can include electrodes, insulating layers, or other elements that can be positioned circumferentially around the core layer, such as a core electrode or core polymer wire.

The term “coupled” as used herein is a broad term, and is to be given its ordinary and customary meaning to a person of ordinary skill in the art (and is not to be limited to a special or customized meaning), and refers without limitation to two or more system elements or components that are configured to be at least one of electrically, mechanically, thermally, operably, chemically or otherwise attached. For example, an element is “coupled” if the element is covalently, communicatively, electrostatically, thermally connected, mechanically connected, magnetically connected, or ionically associated with, or physically entrapped, adsorbed to or absorbed by another element. Similarly, the phrases “operably connected”, “operably linked”, and “operably coupled” as used herein may refer to one or more components linked to another component(s) in a manner that facilitates transmission of at least one signal between the components. In some examples, components are part of the same structure and/or integral with one another as in covalently, electrostatically, mechanically, thermally, magnetically, ionically associated with, or physically entrapped, or absorbed (i.e. “directly coupled” as in no intervening element(s)). In other examples, components are connected via remote means. For example, one or more electrodes can be used to detect an analyte in a sample and convert that information into a signal; the signal can then be transmitted to an electronic circuit. In this example, the electrode is “operably linked” to the electronic circuit. The phrase “removably coupled” as used herein may refer to two or more system elements or components that are configured to be or have been electrically, mechanically, thermally, operably, chemically, or otherwise attached and detached without damaging any of the coupled elements or components. The phrase “permanently coupled” as used herein may refer to two or more system elements or components that are configured to be or have been electrically, mechanically, thermally, operably, chemically, or otherwise attached but cannot be uncoupled without damaging at least one of the coupled elements or components, covalently, electrostatically, ionically associated with, or physically entrapped, or absorbed.

The term “discontinuous” as used herein is a broad term, and is to be given its ordinary and customary meaning to a person of ordinary skill in the art (and is not to be limited to a special or customized meaning), and refers without limitation to disconnected, interrupted, or separated portions, layers, coatings, or domains.

The term “distal” as used herein is a broad term, and is to be given its ordinary and customary meaning to a person of ordinary skill in the art (and is not to be limited to a special or customized meaning), and refers without limitation to a region spaced relatively far from a point of reference, such as an origin or a point of attachment.

The term “domain” as used herein is a broad term, and is to be given its ordinary and customary meaning to a person of ordinary skill in the art (and is not to be limited to a special or customized meaning), and refers without limitation to a region of a membrane system that can be a layer, a uniform or non-uniform gradient (for example, an anisotropic region of a membrane), or a portion of a membrane that is capable of sensing one, two, or more analytes. The domains discussed herein can be formed as a single layer, as two or more layers, as pairs of bi-layers, or as combinations thereof.

The term “electrochemically reactive surface” as used herein is a broad term, and is to be given its ordinary and customary meaning to a person of ordinary skill in the art (and is not to be limited to a special or customized meaning), and refers without limitation to the surface of an electrode where an electrochemical reaction takes place. In one example this reaction is faradaic and results in charge transfer between the surface and its environment. In one example, hydrogen peroxide produced by an enzyme-catalyzed reaction of an analyte being oxidized on the surface results in a measurable electronic current. For example, in the detection of glucose, glucose oxidase produces hydrogen peroxide (H₂O₂) as a byproduct. The H₂O₂ reacts with the surface of the working electrode to produce two protons (2H⁺), two electrons (2e⁻) and one molecule of oxygen (O₂), which produces the electronic current being detected. In a counter electrode, a reducible species, for example, O₂ is reduced at the electrode surface so as to balance the current generated by the working electrode.

The term “electrolysis” as used herein is a broad term, and is to be given its ordinary and customary meaning to a person of ordinary skill in the art (and is not to be limited to a special or customized meeting), and refers without limitation to electrooxidation or electroreduction (collectively, “redox”) of a compound, either directly or indirectly, by one or more enzymes, cofactors, or mediators.

The terms “indwelling,” “in dwelling,” “implanted,” or “implantable” as used herein are broad terms, and are to be given their ordinary and customary meaning to a person of ordinary skill in the art (and are not to be limited to a special or customized meaning), and refer without limitation to objects including sensors that are inserted, or configured to be inserted, subcutaneously (i.e. in the layer of fat between the skin and the muscle), intracutaneously (i.e. penetrating the stratum corneum and positioning within the epidermal or dermal strata of the skin), or transcutaneously (i.e. penetrating, entering, or passing through intact skin), which may result in a sensor that has an in vivo portion and an ex vivo portion. The term “indwelling” also encompasses an object which is configured to be inserted subcutaneously, intracutaneously, or transcutaneously, whether or not it has been inserted as such.

The terms “interferants” and “interfering species” as used herein are broad terms, and are to be given their ordinary and customary meaning to a person of ordinary skill in the art (and are not to be limited to a special or customized meaning), and refer without limitation to effects and/or species that interfere with the measurement of an analyte of interest in a sensor to produce a signal that does not accurately represent the analyte measurement. In one example of an electrochemical sensor, interfering species are compounds which produce a signal that is not analyte-specific due to a reaction on an electrochemically active surface.

The term “in vivo” as used herein is a broad term, and is to be given its ordinary and customary meaning to a person of ordinary skill in the art (and is not to be limited to a special or customized meaning), and without limitation is inclusive of the portion of a device (for example, a sensor) adapted for insertion into and/or existence within a living body of a patient.

The term “ex vivo” as used herein is a broad term, and is to be given its ordinary and customary meaning to a person of ordinary skill in the art (and is not to be limited to a special or customized meaning), and without limitation is inclusive of a portion of a device (for example, a sensor) adapted to remain and/or exist outside of a living body of a patient.

The term and phrase “mediator” and “redox mediator” as used herein are broad terms and phrases, and are to be given their ordinary and customary meaning to a person of ordinary skill in the art (and is not to be limited to a special or customized meaning), and refers without limitation to any chemical compound or collection of compounds capable of electron transfer, either directly, or indirectly, between an analyte, analyte precursor, analyte surrogate, analyte-reduced or analyte-oxidized enzyme, or cofactor, and an electrode surface held at a potential. In one example the mediator accepts electrons from, or transfer electrons to, one or more enzymes or cofactors, and/or exchanges electrons with the sensor system electrodes. In one example, mediators are transition-metal coordinated organic molecules which are capable of reversible oxidation and reduction reactions. In other examples, mediators may be organic molecules or metals which are capable of reversible oxidation and reduction reactions.

The term “membrane” as used herein is a broad term, and is to be given its ordinary and customary meaning to a person of ordinary skill in the art (and is not to be limited to a special or customized meaning), and refers without limitation to a structure configured to perform functions including, but not limited to, protection of the exposed electrode surface from the biological environment, diffusion resistance (limitation) of the analyte, service as a matrix for a catalyst (e.g., one or more enzymes) for enabling an enzymatic reaction, limitation or blocking of interfering species, provision of hydrophilicity at the electrochemically reactive surfaces of the sensor interface, service as an interface between patient tissue and the implantable device, modulation of patient tissue response via drug (or other substance) release, and combinations thereof. When used herein, the terms “membrane” and “matrix” are meant to be interchangeable.

The phrase “membrane system” as used herein is a broad phrase, and is to be given its ordinary and customary meaning to a person of ordinary skill in the art (and is not to be limited to a special or customized meaning), and refers without limitation to a permeable or semi-permeable membrane that can be comprised of two or more domains, layers, or layers within a domain, and is typically constructed of materials of a few microns thickness or more, which is permeable to oxygen and is optionally permeable to, e.g., glucose or another analyte. In one example, the membrane system comprises an enzyme, which enables an analyte reaction to occur whereby a concentration of the analyte can be measured.

The term “planar” as used herein is to be interpreted broadly to describe sensor architecture having a substrate including at least a first surface and an opposing second surface, and for example, comprising a plurality of elements arranged on one or more surfaces or edges of the substrate. The plurality of elements can include conductive or insulating layers or elements configured to operate as a circuit. The plurality of elements may or may not be electrically or otherwise coupled. In one example, planar includes one or more edges separating the opposed surfaces.

The term “proximal” as used herein is a broad term, and is to be given its ordinary and customary meaning to a person of ordinary skill in the art (and is not to be limited to a special or customized meaning), and refers without limitation to the spatial relationship between various elements in comparison to a particular point of reference. For example, some examples of a device include a membrane system having a biointerface layer and an enzyme domain or layer. If the sensor is deemed to be the point of reference and the enzyme domain is positioned nearer to the sensor than the biointerface layer, then the enzyme domain is more proximal to the sensor than the biointerface layer.

The phrases “sensing portion,” “sensing membrane,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In one example, the sensing portion, sensing membrane, and/or sensing mechanism generally comprise an electrode configured to provide signals during electrochemically reactions with one or more membranes covering electrochemically reactive surface. In one example, such sensing portions, sensing membranes, and/or sensing mechanisms are capable of providing specific quantitative, semi-quantitative, qualitative, semi qualitative analytical signals using a biological recognition element combined with a transducing and/or detecting element.

During general operation of the analyte measuring device, biosensor, sensor, sensing region, sensing portion, or sensing mechanism, a biological sample, for example, blood or interstitial fluid, or a component thereof contacts, either directly, or after passage through one or more membranes, an enzyme, for example, glucose oxidase, DNA, RNA, or a protein or aptamer, for example, one or more periplasmic binding protein (PBP) or mutant or fusion protein thereof having one or more analyte binding regions, each region capable of specifically or reversibly binding to and/or reacting with at least one analyte. The interaction of the biological sample or component thereof with the analyte measuring device, biosensor, sensor, sensing region, sensing portion, or sensing mechanism results in transduction of a signal that permits a qualitative, semi-qualitative, quantitative, or semi-qualitative determination of the analyte level, for example, glucose, ketone, lactate, potassium, etc., in the biological sample.

In one example, the sensing region or sensing portion can comprise at least a portion of a conductive substrate or at least a portion of a conductive surface, for example, a wire (coaxial) or conductive trace or a substantially planar substrate including substantially planar trace(s), and a membrane. In one example, the sensing region or sensing portion can comprise a non-conductive body, a working electrode, a reference electrode, and a counter electrode (optional), forming an electrochemically reactive surface at one location on the body and an electronic connection at another location on the body, and a sensing membrane affixed to the body and covering the electrochemically reactive surface. In some examples, the sensing membrane further comprises an enzyme domain, for example, an enzyme domain, and an electrolyte phase, for example, a free-flowing liquid phase comprising an electrolyte-containing fluid described further below. The terms are broad enough to include the entire device, or only the sensing portion thereof (or something in between).

In another example, the sensing region can comprise one or more periplasmic binding protein (PBP) including mutant or fusion protein thereof, or aptamers having one or more analyte binding regions, each region capable of specifically and reversibly binding to at least one analyte. Alterations of the aptamer or mutations of the PBP can contribute to or alter one or more of the binding constants, long-term stability of the protein, including thermal stability, to bind the protein to a special encapsulation matrix, membrane or polymer, or to attach a detectable reporter group or “label” to indicate a change in the binding region or transduce a signal corresponding to the one or more analytes present in the biological fluid. Specific examples of changes in the binding region include, but are not limited to, hydrophobic/hydrophilic environmental changes, three-dimensional conformational changes, changes in the orientation of amino/nucleic acid side chains in the binding region of proteins, and redox states of the binding region. Such changes to the binding region provide for transduction of a detectable signal corresponding to the one or more analytes present in the biological fluid.

In one example, the sensing region determines the selectivity among one or more analytes, so that only the analyte which has to be measured leads to (transduces) a detectable signal. The selection may be based on any chemical or physical recognition of the analyte by the sensing region, where the chemical composition of the analyte is unchanged, or in which the sensing region causes or catalyzes a reaction of the analyte that changes the chemical composition of the analyte.

The term “sensitivity” as used herein is a broad term, and is to be given its ordinary and customary meaning to a person of ordinary skill in the art (and is not to be limited to a special or customized meaning), and refers without limitation to an amount of signal (e.g., in the form of electrical current and/or voltage) produced by a predetermined amount (unit) of the measured analyte. For example, in one example, a sensor has a sensitivity (or slope) of from about 1 to about 100 picoAmps of current for every 1 mg/dL of analyte.

The phrases “signal medium” or “transmission medium” shall be taken to include any form of modulated data signal, carrier wave, and so forth. The phrase “modulated data signal” means a signal that has one or more of its characteristics set or changed in such a matter as to encode information in the signal.

The terms “transducing” or “transduction” and their grammatical equivalents as are used herein are broad terms, and are to be given their ordinary and customary meaning to a person of ordinary skill in the art (and is not to be limited to a special or customized meaning), and refer without limitation to optical, electrical, electrochemical, acoustical/mechanical, or colorimetrical technologies and methods. Electrochemical properties include current and/or voltage, inductance, capacitance, impedance, transconductance, and potential. Optical properties include absorbance, fluorescence/phosphorescence, fluorescence/phosphorescence decay rate, wavelength shift, dual wave phase modulation, bio/chemiluminescence, reflectance, light scattering, and refractive index. For example, the sensing region transduces the recognition of analytes into a semi-quantitative or quantitative signal.

As used herein, the phrase “transducing element” as used herein is a broad phrase, and are to be given their ordinary and customary meaning to a person of ordinary skill in the art (and is not to be limited to a special or customized meaning), and refers without limitation to analyte recognition moieties capable of facilitating, directly or indirectly, with detectable signal transduction corresponding to the presence and/or concentration of the recognized analyte. In one example, a transducing element is one or more enzymes, one or more aptamers, one or more ionophores, one or more capture antibodies, one or more proteins, one or more biological cells, one or more oligonucleotides, and/or one or more DNA or RNA moieties. Transcutaneous continuous multi-analyte sensors can be used in vivo over various lengths of time. The continuous multi-analyte sensor systems discussed herein can be transcutaneous devices, in that a portion of the device may be inserted through the patient's skin and into the underlying soft tissue while a portion of the device remains on the surface of the patient's skin. In one aspect, in order to overcome the problems associated with noise or other sensor function in the short-term, one example employs materials that promote formation of a fluid pocket around the sensor, for example architectures such as a porous biointerface membrane or matrices that create a space between the sensor and the surrounding tissue. In some examples, a sensor is provided with a spacer adapted to provide a fluid pocket between the sensor and the patient's tissue. It is believed that this spacer, for example a biointerface material, matrix, structure, and the like as described in more detail elsewhere herein, provides for oxygen and/or glucose transport to the sensor.

### Membrane Systems

Membrane systems disclosed herein are suitable for use with implantable devices in contact with a biological fluid. For example, the membrane systems can be utilized with implantable devices, such as devices for monitoring and determining analyte levels in a biological fluid, for example, devices for monitoring glucose levels for individuals having diabetes. In some examples, the analyte-measuring device is a continuous device. The analyte-measuring device can employ any suitable sensing element to provide the raw signal, including but not limited to those involving enzymatic, chemical, physical, electrochemical, spectrophotometric, amperometric, potentiometric, polarimetric, calorimetric, radiometric, immunochemical, or like elements.

Suitable membrane systems for the aforementioned multi-analyte systems and devices can include, for example, membrane systems disclosed in U.S. Pat. Nos. 6,015,572, 5,964,745, and 6,083,523, which are incorporated herein by reference in their entireties for their teachings of membrane systems.

In general, the membrane system includes a plurality of domains, for example, an electrode domain, an interference domain, an enzyme domain, a resistance domain, and a biointerface domain. The membrane system can be deposited on the exposed electroactive surfaces using known thin film techniques (for example, vapor deposition, spraying, electrodepositing, dipping, brush coating, film coating, drop-let coating, and the like). Additional steps may be applied following the membrane material deposition, for example, drying, annealing, and curing (for example, UV curing, thermal curing, moisture curing, radiation curing, and the like) to enhance certain properties such as mechanical properties, signal stability, and selectivity. In a typical process, upon deposition of the resistance domain membrane, a biointerface/drug releasing layer having a “dry film” thickness of from about 0.05 micron (m), or less, to about 1, 2, 3, 4, 5, 6, 7, 8, 9, 10, 11, 12, 13, 14, 15, or 16 m is formed. “Dry film” thickness refers to the thickness of a cured film cast from a coating formulation by standard coating techniques.

In certain examples, the biointerface/drug releasing layer is formed of a biointerface polymer, wherein the biointerface polymer comprises one or more membrane domains comprising polyurethane and/or polyurea segments and one or more zwitterionic repeating units. In some examples, the biointerface/drug releasing layer coatings are formed of a polyurethane urea having carboxyl betaine groups incorporated in the polymer and non-ionic hydrophilic polyethylene oxide segments, wherein the polyurethane urea polymer is dissolved in organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the bioprotective polymers are formed of a polyurethane urea having carboxylic acid groups and carboxyl betaine groups incorporated in the polymer and non-ionic hydrophilic polyethylene oxide segments, wherein the polyurethane urea polymer is dissolved in an organic or non-organic solvent system in a coating formulation, and is crosslinked with an a carbodiimide (e.g., 1-ethyl-3-(3-dimethylaminopropyl)carbodiimide (EDC)) and cured at a moderate temperature of about 50° C.

In other examples, the biointerface/drug releasing layer coatings are formed of a polyurethane urea having sulfobetaine groups incorporated in the polymer and non-ionic hydrophilic polyethylene oxide segments, wherein the polyurethane urea polymer is dissolved in an organic or non-organic solvent system according to a pre-determined coating formulation, and is crosslinked with an isocyanate crosslinker and cured at a moderate temperature of about 50° C. The solvent system can be a single solvent or a mixture of solvents to aid the dissolution or dispersion of the polymer. The solvents can be the ones selected as the polymerization media or added after polymerization is completed. The solvents are selected from the ones having lower boiling points to facilitate drying and to be lower in toxicity for implant applications. Examples of these solvents include aliphatic ketone, ester, ether, alcohol, hydrocarbons, and the like. Depending on the final thickness of the biointerface/drug releasing layer and solution viscosity (as related to the percent of polymer solid), the coating can be applied in a single step or multiple repeated steps of the chosen process such as dipping to build the desired thickness. Yet in other examples, the biointerface polymers are formed of a polyurethane urea having unsaturated hydrocarbon groups and sulfobetaine groups incorporated in the polymer and non-ionic hydrophilic polyethylene oxide segments, wherein the polyurethane urea polymer is dissolved in an organic or non-organic solvent system in a coating formulation, and is crosslinked in the presence of initiators with heat or irradiation including UV, LED light, electron beam, and the like, and cured at a moderate temperature of about 50° C. Examples of unsaturated hydrocarbon includes allyl groups, vinyl groups, acrylate, methacrylate, alkenes, alkynes, and the like.

In some examples, tethers are used. A tether is a polymer or chemical moiety which does not participate in the (electro)chemical reactions involved in sensing, but forms chemical bonds with the (electro)chemically active components of the membrane. In some examples these bonds are covalent. In one example, a tether may be formed in solution prior to one or more interlayers of a membrane being formed, where the tether bonds two (electro)chemically active components directly to one another or alternately, the tether(s) bond (electro)chemically active component(s) to polymeric backbone structures. In another example, (electro)chemically active components are comixed along with crosslinker(s) with tunable lengths (and optionally polymers) and the tethering reaction occurs as in situ crosslinking. Tethering may be employed to maintain a predetermined number of degrees of freedom of NAD(P)H for effective enzyme catalysis, where “effective” enzyme catalysis causes the analyte sensor to continuously monitor one or more analytes for a period of from about 5 days to about 15 days or more.

### Membrane Fabrication

Polymers can be processed by solution-based techniques such as spraying, dipping, casting, electrospinning, vapor deposition, spin coating, coating, and the like. Water-based polymer emulsions can be fabricated to form membranes by methods similar to those used for solvent-based materials. In both cases the evaporation of a volatile liquid (e.g., organic solvent or water) leaves behind a film of the polymer. Cross-linking of the deposited film or layer can be performed through the use of multi-functional reactive ingredients by a number of methods. The liquid system can cure by heat, moisture, high-energy radiation, ultraviolet light, or by completing the reaction, which produces the final polymer in a mold or on a substrate to be coated.

In some examples, the wetting property of the membrane (and by extension the extent of sensor drift exhibited by the sensor) can be adjusted and/or controlled by creating covalent cross-links between surface-active group-containing polymers, functional-group containing polymers, polymers with zwitterionic groups (or precursors or derivatives thereof), and combinations thereof. Cross-linking can have a substantial effect on film structure, which in turn can affect the film's surface wetting properties. Crosslinking can also affect the film's tensile strength, mechanical strength, water absorption rate and other properties.

Cross-linked polymers can have different cross-linking densities. In certain examples, cross-linkers are used to promote cross-linking between layers. In other examples, in replacement of (or in addition to) the cross-linking techniques described above, heat is used to form cross-linking. For example, in some examples, imide and amide bonds can be formed between two polymers as a result of high temperature. In some examples, photo cross-linking is performed to form covalent bonds between the polycationic layers(s) and polyanionic layer(s). One major advantage to photo-cross-linking is that it offers the possibility of patterning. In certain examples, patterning using photo-cross linking is performed to modify the film structure and thus to adjust the wetting property of the membranes and membrane systems, as discussed herein.

Polymers with domains or segments that are functionalized to permit cross-linking can be made by methods at least as discussed herein. For example, polyurethaneurea polymers with aromatic or aliphatic segments having electrophilic functional groups (e.g., carbonyl, aldehyde, anhydride, ester, amide, isocyano, epoxy, allyl, or halo groups) can be crosslinked with a crosslinking agent that has multiple nucleophilic groups (e.g., hydroxyl, amine, urea, urethane, or thiol groups). In further examples, polyurethaneurea polymers having aromatic or aliphatic segments having nucleophilic functional groups can be crosslinked with a crosslinking agent that has multiple electrophilic groups. Still further, polyurethaneurea polymers having hydrophilic segments having nucleophilic or electrophilic functional groups can be crosslinked with a crosslinking agent that has multiple electrophilic or nucleophilic groups. Unsaturated functional groups on the polyurethane urea can also be used for crosslinking by reacting with multivalent free radical agents. Non-limiting examples of suitable cross-linking agents include isocyanate, carbodiimide, glutaraldehyde, aziridine, silane, or other aldehydes, epoxy, acrylates, free-radical based agents, ethylene glycol diglycidyl ether (EGDE), poly(ethylene glycol) diglycidyl ether (PEGDE), or dicumyl peroxide (DCP). In one example, from about 0.1% to about 15% w/w of cross-linking agent is added relative to the total dry weights of cross-linking agent and polymers added when blending the ingredients. In another example, about 1% to about 10% w/w of cross-linking agent is added relative to the total dry weights of cross-linking agent and polymers added when blending the ingredients. In yet another example, about 5% to about 15% w/w of cross-linking agent is added relative to the total dry weights of cross-linking agent and polymers added when blending the ingredients. During the curing process, substantially all of the cross-linking agent is believed to react, leaving substantially no detectable unreacted cross-linking agent in the final film.

Polymers disclosed herein can be formulated into mixtures that can be drawn into a film or applied to a surface using methods such as spraying, self-assembling monolayers (SAMs), painting, dip coating, vapor depositing, molding, 3-D printing, lithographic techniques (e.g., photolithograph), micro- and nano-pipetting printing techniques, silk-screen printing, etc.). The mixture can then be cured under high temperature (e.g., from about 30° C. to about 150° C.). Other suitable curing methods can include ultraviolet, e-beam, or gamma radiation, for example.

In some circumstances, using continuous multianalyte monitoring systems including sensor(s) configured with bioprotective and/or drug releasing membranes, it is believed that that foreign body response is the dominant event surrounding extended implantation of an implanted device and can be managed or manipulated to support rather than hinder or block analyte transport. In another aspect, in order to extend the lifetime of the sensor, one example employs materials that promote vascularized tissue ingrowth, for example within a porous biointerface membrane. For example, tissue in-growth into a porous biointerface material surrounding a sensor may promote sensor function over extended periods of time (e.g., weeks, months, or years). It has been observed that in-growth and formation of a tissue bed can take up to 3 weeks. Tissue ingrowth and tissue bed formation is believed to be part of the foreign body response. As will be discussed herein, the foreign body response can be manipulated by the use of porous bioprotective materials that surround the sensor and promote ingrowth of tissue and microvasculature over time.

Accordingly, a sensor as discussed in examples herein may include a biointerface layer. The biointerface layer, like the drug releasing layer, may include, but is not limited to, for example, porous biointerface materials including a solid portion and interconnected cavities, all of which are described in more detail elsewhere herein. The biointerface layer can be employed to improve sensor function in the long term (e.g., after tissue ingrowth).

Accordingly, a sensor as discussed in examples herein may include a drug releasing membrane at least partially functioning as or in combination with a biointerface membrane. The drug releasing membrane may include, for example, materials including a hard-soft segment polymer with hydrophilic and optionally hydrophobic domains, all of which are described in more detail elsewhere herein, can be employed to improve sensor function in the long term (e.g., after tissue ingrowth). In one example, the materials including a hard-soft segment polymer with hydrophilic and optionally hydrophobic domains are configured to release a combination of a derivative form of dexamethasone or dexamethasone acetate with dexamethasone such that one or more different rates of release of the anti-inflammatory is achieved and the useful life of the sensor is extended. Other suitable drug releasing membranes of the present disclosure can be selected from silicone polymers, polytetrafluoroethylene, expanded polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polyvinyl alcohol (PVA), poly vinyl acetate, ethylene vinyl acetate (EVA), polybutylene terephthalate (PBT), polymethylmethacrylate (PMMA), polyether ether ketone (PEEK), polyamides, polyurethanes and copolymers and blends thereof, polyurethane urea polymers and copolymers and blends thereof, cellulosic polymers and copolymers and blends thereof, poly(ethylene oxide) and copolymers and blends thereof, poly(propylene oxide) and copolymers and blends thereof, polysulfones and block copolymers thereof including, for example, di-block, tri-block, alternating, random and graft copolymers cellulose, hydrogel polymers, poly(2-hydroxyethyl methacrylate, pHEMA) and copolymers and blends thereof, hydroxyethyl methacrylate, (HEMA) and copolymers and blends thereof, polyacrylonitrile-polyvinyl chloride (PAN-PVC) and copolymers and blends thereof, acrylic copolymers and copolymers and blends thereof, nylon and copolymers and blends thereof, polyvinyl difluoride, polyanhydrides, poly(l-lysine), poly(L-lactic acid), hydroxyethylmetharcrylate and copolymers and blends thereof, and hydroxyapeptite and copolymers and blends thereof.

### Exemplary Multi-Analyte Sensor Membrane Configurations

Continuous multi-analyte sensors with various membrane configurations suitable for facilitating signal transduction corresponding to analyte concentrations, either simultaneously, intermittently, and/or sequentially are provided. In one example, such sensors can be configured using a signal transducer, comprising one or more transducing elements (“TL”). Such continuous multi-analyte sensor can employ various transducing means, for example, amperometry, voltametric, potentiometry, and impedimetric methods, among other techniques.

In one example, the transducing element comprises one or more membranes that can comprise one or more layers and or domains, each of the one or more layers or domains can independently comprise one or more signal transducers, e.g., enzymes, RNA, DNA, aptamers, binding proteins, etc. As used herein, transducing elements includes enzymes, ionophores, RNA, DNA, aptamers, binding proteins and are used interchangeably.

In one example, the transducing element is present in one or more membranes, layers, or domains formed over a sensing region. In one example, such sensors can be configured using one or more enzyme domains, e.g., membrane domains including enzyme domains, also referred to as EZ layers (“EZLs”), each enzyme domain may comprise one or more enzymes. Reference hereinafter to an “enzyme layer” is intended to include all or part of an enzyme domain, either of which can be all or part of a membrane system as discussed herein, for example, as a single layer, as two or more layers, as pairs of bi-layers, or as combinations thereof.

In one example, the continuous multi-analyte sensor uses one or more of the following analyte-substrate/enzyme pairs: for example, sarcosine oxidase in combination with creatinine amidohydrolase, creatine amidohydrolase being employed for the sensing of creatinine. Other examples of analytes/oxidase enzyme combinations that can be used in the sensing region include, for example, alcohol/alcohol oxidase, cholesterol/cholesterol oxidase, glactose:galactose/galactose oxidase, choline/choline oxidase, glutamate/glutamate oxidase, glycerol/glycerol-3phosphate oxidase (or glycerol oxidase), bilirubin/bilirubin oxidase, ascorbic/ascorbic acid oxidase, uric acid/uric acid oxidase, pyruvate/pyruvate oxidase, hypoxanthine:xanthine/xanthine oxidase, glucose/glucose oxidase, lactate/lactate oxidase, L-amino acid oxidase, and glycine/sarcosine oxidase. Other analyte-substrate/enzyme pairs can be used, including such analyte-substrate/enzyme pairs that comprise genetically altered enzymes, immobilized enzymes, mediator-wired enzymes, dimerized and/or fusion enzymes.

### NAD Based Multi-Analyte Sensor Platform

Nicotinamide adenine dinucleotide (NAD(P)+/NAD(P)H) is a coenzyme, e.g., a dinucleotide that consists of two nucleotides joined through their phosphate groups. One nucleotide contains an adenine nucleobase and the other nicotinamide. NAD exists in two forms, e.g., an oxidized form (NAD(P)+) and reduced form (NAD(P)H) (H=hydrogen). The reaction of NAD+ and NADH is reversible, thus, the coenzyme can continuously cycle between the NAD(P)+/and NAD(P)H forms essentially without being consumed.

In one example, one or more enzyme domains of the sensing region of the presently disclosed continuous multi-analyte sensor device comprise an amount of NAD+ or NADH for providing transduction of a detectable signal corresponding to the presence or concentration of one or more analytes. In one example, one or more enzyme domains of the sensing region of the presently disclosed continuous multi-analyte sensor device comprise an excess amount of NAD+ or NADH for providing extended transduction of a detectable signal corresponding to the presence or concentration of one or more analytes.

In one example, NAD, NADH, NAD+, NAD(P)+, ATP, flavin adenine dinucleotide (FAD), magnesium (Mg++), pyrroloquinoline quinone (PQQ), and functionalized derivatives thereof can be used in combination with one or more enzymes in the continuous multi-analyte sensor device. In one example, NAD, NADH, NAD+, NAD(P)+, ATP, flavin adenine dinucleotide (FAD), magnesium (Mg++), pyrroloquinoline quinone (PQQ), and functionalized derivatives are incorporated in the sensing region. In one example, NAD, NADH, NAD+, NAD(P)+, ATP, flavin adenine dinucleotide (FAD), magnesium (Mg++), pyrroloquinoline quinone (PQQ), and functionalized derivatives are dispersed or distributed in one or more membranes or domains of the sensing region.

In one aspect of the present disclosure, continuous sensing of one or more or two or more analytes using NAD+ dependent enzymes is provided in one or more membranes or domains of the sensing region. In one example, the membrane or domain provides retention and stable recycling of NAD+ as well as mechanisms for transducing NADH oxidation or NAD+ reduction into measurable current with amperometry. In one example, described below, continuous, sensing of multi-analytes, either reversibly bound or at least one of which are oxidized or reduced by NAD+ dependent enzymes, for example, ketones (beta-hydroxybutyrate dehydrogenase), glycerol (glycerol dehydrogenase), cortisol (11β-hydroxysteroid dehydrogenase), glucose (glucose dehydrogenase), alcohol (alcohol dehydrogenase), aldehydes (aldehyde dehydrogenase), and lactate (lactate dehydrogenase) is provided. In other examples, described below, membranes are provided that enable the continuous, on-body sensing of multiple analytes which utilize FAD-dependent dehydrogenases, such as fatty acids (Acyl-CoA dehydrogenase).

Exemplary configurations of one or more membranes or portions thereof are an arrangement for providing retention and recycling of NAD+ are provided. Thus, an electrode surface of a conductive wire (coaxial) or a planar conductive surface is coated with at least one layer comprising at least one enzyme as depicted in FIG. 9A. With reference to FIG. 9B, one or more optional layers may be positioned between the electrode surface and the one or more enzyme domains. For example, one or more interference domains (also referred to as “interferent blocking layer”) can be used to reduce or eliminate signal contribution from undesirable species present, or one or more electrodes (not shown) can used to assist with wetting, system equilibrium, and/or start up. As shown in FIGS. 9A-9B, one or more of the membranes provides a NAD+ reservoir domain providing a reservoir for NAD+. In one example, one or more interferent blocking membranes is used, and potentiostat is utilized to measure H₂O₂ production or O2 consumption of an enzyme such as or similar to NADH oxidase, the NAD+ reservoir and enzyme domain positions can be switched, to facilitate better consumption and slower unnecessary outward diffusion of excess NAD+. Exemplary sensor configurations can be found in U.S. Provisional Patent Application No. 63/321,340, “CONTINUOUS ANALYTE MONITORING SENSOR SYSTEMS AND METHODS OF USING THE SAME,” filed Mar. 18, 2022, and incorporated by reference in its entirety herein.

In one example, one or more mediators that are optimal for NADH oxidation are incorporated in the one or more electrode domains or enzyme domains. In one example, organic mediators, such as phenanthroline dione, or nitrosoanilines are used. In another example, metallo-organic mediators, such as ruthenium-phenanthroline-dione or osmium(bpy)₂Cl, polymers containing covalently coupled organic mediators or organometallic coordinated mediators polymers for example polyvinylimidizole-Os(bpy)₂Cl, or polyvinylpyridine-organometallic coordinated mediators (including ruthenium-phenanthroline dione) are used. Other mediators can be used as discussed further below.

In humans, serum levels of beta-hydroxybutyrate (BHB) are usually in the low micromolar range but can rise up to about 6-8 mM. Serum levels of BHB can reach 1-2 mM after intense exercise or consistent levels above 2 mM are reached with a ketogenic diet that is almost devoid of carbohydrates. Other ketones are present in serum, such as acetoacetate and acetone, however, most of the dynamic range in ketone levels is in the form of BHB. Thus, monitoring of BHB, e.g., continuous monitoring is useful for providing health information to a patient or health care provider.

Another example of a continuous ketone analyte detection configuration employing electrode-associated mediator-coupled diaphorase/NAD+/dehydrogenase is depicted below:

In one example, the diaphorase is electrically coupled to the electrode with organometallic coordinated mediator polymer. In another example, the diaphorase is covalently coupled to the electrode with an organometallic coordinated mediator polymer. Alternatively, multiple enzyme domains can be used in an enzyme layer, for example, separating the electrode-associated diaphorase (closest to the electrode surface) from the more distal adjacent NAD+ or the dehydrogenase enzyme, to essentially decouple NADH oxidation from analyte (ketone) oxidation. Alternatively, NAD+ can be more proximal to the electrode surface than an adjacent enzyme domain comprising the dehydrogenase enzyme. In one example, the NAD+ and/or HBDH are present in the same or different enzyme domain, and either can be immobilized, for example, using amine reactive crosslinker (e.g., glutaraldehyde, epoxides, NHS esters, imidoesters). In one example, the NAD+ is coupled to a polymer and is present in the same or different enzyme domain as HBDH. In one example, the molecular weight of NAD+ is increased to prevent or eliminate migration from the sensing region, for example the NAD+ is dimerized using its C6 terminal amine with any amine-reactive crosslinker. In one example, NAD+ may be covalently coupled to an aspect of the enzyme domain having a higher molecular weight than the NAD+ which may improve a stability profile of the NAD+, improving the ability to retain and/or immobilize the NAD+ in the enzyme domain. For example, dextran-NAD.

In one example, the sensing region comprises one or more NADH:acceptor oxidoreductases and one or more NAD-dependent dehydrogenases. In one example, sensing region comprises one or more NADH:acceptor oxidoreductases and one or more NAD(P)-dependent dehydrogenases with NAD(P)+ or NAD(P)H as cofactors present in sensing region. In one example, the sensing region comprises an amount of diaphorase.

In one example, a ketone sensing configuration suitable for combination with another analyte sensing configuration is provided. Thus, an EZL layer of about 1-20 um thick is prepared by presenting a EZL solution composition in 10 mM HEPES in water having about 20 uL 500 mg/mL HBDH, about 20 uL [500 mg/mL NAD(P)H, 200 mg/mL polyethylene glycol-diglycol ether (PEG-DGE) of about 400 MW], about 20 uL 500 mg/mL diaphorase, about 40 uL 250 mg/mL poly vinyl imidazole-osmium bis(2,2′-bipyridine)chloride (PVI-Os(bpy)2Cl) to a substrate such as a working electrode, so as to provide, after drying, about 15-40% by weight HBDH, about 5-30% diaphorase about 5-30% NAD(P)H, about 10-50% PVI-Os(bpy)2Cl and about 1-12% PEG-DGE(400 MW). The substrates discussed herein that may include working electrodes may be formed from gold, platinum, palladium, rhodium, iridium, titanium, tantalum, chromium, and/or alloys or combinations thereof, or carbon (e.g., graphite, glassy carbon, carbon nanotubes, graphene, or doped diamond, as well combinations thereof.

To the above enzyme domain was contacted a resistance domain, also referred to as a resistance layer (“RL”). In one example, the RL comprises about 55-100% PVP, and about 0.1-45% PEG-DGE. In another example, the RL comprises about 75-100% PVP, and about 0.3-25% PEG-DGE. In yet another example, the RL comprises about 85-100% PVP, and about 0.5-15% PEG-DGE. In yet another example, the RL comprises essentially 100% PVP.

The exemplary continuous ketone sensor as depicted in FIGS. 9A-9B comprising NAD(P)H reservoir domain is configured so that NAD(P)H is not rate-limiting in any of the enzyme domains of the sensing region. In one example, the loading of NAD(P)H in the NAD(P)H reservoir domain is greater than about 20%, 30%, 40% or 50% w/w. The one or more of the membranes or portions of one or more membrane domains (hereinafter also referred to as “membranes”) may also contain a polymer or protein binder, such as zwitterionic polyurethane, and/or albumin. Alternatively, in addition to NAD(P)H, the membrane may contain one or more analyte specific enzymes (e.g. HBDH, glycerol dehydrogenase, etc.), so that optionally, the NAD(P)H reservoir membrane also provides a catalytic function. In one example, the NAD(P)H is dispersed or distributed in or with a polymer (or protein), and may be crosslinked to an extent that still allows adequate enzyme/cofactor functionality and/or reduced NAD(P)H flux within the domain.

In one example, NADH oxidase enzyme alone or in combination with superoxide dismutase (SOD) is used in the one or more membranes of the sensing region. In one example, an amount of superoxide dismutase (SOD) is used that is capable of scavenging some or most of one or more free radicals generated by NADH oxidase. In one example, NADH oxidase enzyme alone or in combination with superoxide dismutase (SOD) is used in combination with NAD(P)H and/or a functionalized polymer with NAD(P)H immobilized onto the polymer from a C6 terminal amine in the one or more membranes of the sensing region.

In one example, the NAD(P)H is immobilized to an extent that maintains NAD(P)H catalytic functionality. In one example, dimerized NAD(P)H is used to entrap NAD(P)H within one or more membranes by crosslinking their respective C6 terminal amine together with appropriate amine-reactive crosslinker such as glutaraldehyde or PEG-DGE.

The aforementioned continuous ketone sensor configurations can be adapted to other analytes or used in combination with other sensor configurations. For example, analyte(s)-dehydrogenase enzyme combinations can be used in any of the membranes of the sensing region include; glycerol (glycerol dehydrogenase); cortisol (11β-hydroxysteroid dehydrogenase); glucose (glucose dehydrogenase); alcohol (alcohol dehydrogenase); aldehydes (aldehyde dehydrogenase); and lactate (lactate dehydrogenase).

In one example, a semipermeable membrane is used in the sensing region or adjacent thereto or adjacent to one or more membranes of the sensing region so as to attenuate the flux of at least one analyte or chemical species. In one example, the semipermeable membrane attenuates the flux of at least one analyte or chemical species so as to provide a linear response from a transduced signal. In another example, the semipermeable membrane prevents or eliminates the flux of NAD(P)H out of the sensing region or any membrane or domain. In one example, the semipermeable membrane can be an ion selective membrane selective for an ion analyte of interest, such as ammonium ion.

In another example, a continuous multi-analyte sensor configuration comprising one or more enzymes and/or at least one cofactor was prepared. In certain embodiments, an enzyme domain 950 comprising an enzyme (Enzyme) with an amount of cofactor (Cofactor) is positioned proximal to at least a portion of a working electrode (“WE”) surface, where the WE comprises an electrochemically reactive surface. In one example, a second membrane 951 comprising an amount of cofactor is positioned adjacent the first enzyme domain. The amount of cofactor in the second membrane can provide an excess for the enzyme, e.g., to extend sensor life. One or more resistance domains 952 (“RL”) are positioned adjacent the second membrane (or can be between the membranes). The RL can be configured to block diffusion of cofactor from the second membrane. Electron transfer from the cofactor to the WE transduces a signal that corresponds directly or indirectly to an analyte concentration.

FIG. 9D depicts an alternative enzyme domain configuration comprising a first membrane 951 with an amount of cofactor that is positioned more proximal to at least a portion of a WE surface. Enzyme domain 950 comprising an amount of enzyme is positioned adjacent the first membrane.

In the membrane configurations depicted in FIGS. 9C-9D, production of an electrochemically active species in the enzyme domain diffuses to the WE surface and transduces a signal that corresponds directly or indirectly to an analyte concentration. In some examples, the electrochemically active species comprises hydrogen peroxide. For sensor configurations that include a cofactor, the cofactor from the first layer can diffuse to the enzyme domain to extend sensor life, for example, by regenerating the cofactor. For other sensor configurations, the cofactor can be optionally included to improve performance attributes, such as stability. For example, a continuous ketone sensor can comprise NAD(P)H and a divalent metal cation, such as Mg⁺². One or more resistance domains RL can be positioned adjacent the second membrane (or can be between the layers). The RL can be configured to block diffusion of cofactor from the second membrane and/or interferents from reaching the WE surface. Other configurations can be used in the aforementioned configuration, such as electrode, resistance, bio-interfacing, and drug releasing membranes, layers or domains. In other examples, continuous analyte sensors including one or more cofactors that contribute to sensor performance.

FIG. 9E depicts another continuous multi-analyte membrane configuration, where {beta}-hydroxybutyrate dehydrogenase BHBDH in a first enzyme domain 953 is positioned proximate to a working electrode WE and second enzyme domain 954, for example, comprising alcohol dehydrogenase (ADH) and NADH is positioned adjacent the first enzyme domain. One or more resistance domains RL 952 may be deployed adjacent to the second enzyme domain 954. In this configuration, the presence of the combination of alcohol and ketone in serum works collectively to provide a transduced signal corresponding to at least one of the analyte concentrations, for example, ketone. Thus, as the NADH present in the more distal second enzyme domain consumes alcohol present in the serum environment, NADH is oxidized to NAD(P)H that diffuses into the first membrane layer to provide electron transfer of the BHBDH catalysis of acetoacetate ketone and transduction of a detectable signal corresponding to the concentration of the ketone. In one example, an enzyme can be configured for reverse catalysis and can create a substrate used for catalysis of another enzyme present, either in the same or different layer or domain. Other configurations can be used in the aforementioned configuration, such as electrode, resistance, bio-interfacing, and drug releasing membranes, layers, or domains. Thus, a first enzyme domain that is more distal from the WE than a second enzyme domain may be configured to generate a cofactor or other element to act as a reactant (and/or a reactant substrate) for the second enzyme domain to detect the one or more target analytes.

### Alcohol Sensor Configurations

In one example, a continuous alcohol (e.g., ethanol) sensor device configuration is provided. In one example, one or more enzyme domains comprising alcohol oxidase (AOX) is provided and the presence and/or amount of alcohol is transduced by creation of hydrogen peroxide, alone or in combination with oxygen consumption or with another substrate-oxidase enzyme system, e.g., glucose-glucose oxidase, in which hydrogen peroxide and or oxygen and/or glucose can be detected and/or measured qualitatively or quantitatively, using amperometry.

In one example, the sensing region for the aforementioned enzyme substrate-oxidase enzyme configurations has one or more enzyme domains comprises one or more electrodes. In one example, the sensing region for the aforementioned enzyme substrate-oxidase enzyme configurations has one or more enzyme domains, with or without the one or more electrodes, further comprises one or interference blocking membranes (e.g. permselective membranes, charge exclusion membranes) to attenuate one or more interferents from diffusing through the membrane to the working electrode. In one example, the sensing region for the aforementioned substrate-oxidase enzyme configurations has one or more enzyme domains, with or without the one or more electrodes, and further comprises one or resistance domains with or without the one or more interference blocking membranes to attenuate one or more analytes or enzyme substrates. In one example, the sensing region for the aforementioned substrate-oxidase enzyme configurations has one or more enzyme domains, with or without the one or more electrodes, one or more resistance domains with or without the one or more interference blocking membranes further comprises one or biointerface membranes and/or drug releasing membranes, independently, to attenuate one or more analytes or enzyme substrates and attenuate the immune response of the patient after insertion.

In one example, the one or more interference blocking membranes are deposited adjacent the working electrode and/or the electrode surface. In one example, the one or interference blocking membranes are directly deposited adjacent the working electrode and/or the electrode surface. In one example, the one or interference blocking membranes are deposited between another layer or membrane or domain that is adjacent the working electrode or the electrode surface to attenuate one or all analytes diffusing thru the sensing region but for oxygen. Such membranes can be used to attenuate alcohol itself as well as attenuate other electrochemically actives species or other analytes that can otherwise interfere by producing a signal if they diffuse to the working electrode.

In one example, the working electrode used comprised platinum and the potential applied was about 0.5 volts.

In one example, sensing oxygen level changes electrochemically, for example in a Clark type electrode setup, or in a different configuration can be carried out, for example by coating the electrode with one or more membranes of one or more polymers, such as NAFION™. Based on changes of potential, oxygen concentration changes can be recorded, which correlate directly or indirectly with the concentrations of alcohol. When appropriately designed to obey stoichiometric behavior, the presence of a specific concentration of alcohol should cause a commensurate reduction in local oxygen in a direct (linear) relation with the concentration of alcohol. Accordingly, a multi-analyte sensor for both alcohol and oxygen can therefore be provided.

In another example, the above mentioned alcohol sensing configuration can include one or more secondary enzymes that react with a reaction product of the alcohol/alcohol oxidase catalysis, e.g., hydrogen peroxide, and provide for a oxidized form of the secondary enzyme that transduces an alcohol-dependent signal to the WE/RE at a lower potential than without the secondary enzyme. Thus, in one example, the alcohol/alcohol oxidase is used with a reduced form of a peroxidase, for example horse radish peroxidase. The alcohol/alcohol oxidase can be in same or different layer as the peroxidase, or they may be spatially separated distally from the electrode surface, for example, the alcohol/alcohol oxidase being more distal from the electrode surface and the peroxidase being more proximal to the electrode surface, or alternatively, the alcohol/alcohol oxidase being more proximal from the electrode surface and the peroxidase being more distal to the electrode surface. In one example, the alcohol/alcohol oxidase, being more distal from the electrode surface and the peroxidase, further includes any combination of electrode, interference, resistance, and biointerface membranes to optimize signal, durability, reduce drift, or extend end of use duration.

In another example, the above mentioned alcohol sensing configuration can include one or more mediators. In one example, the one or more mediators are present in, on, or about one or more electrodes or electrode surfaces and/or are deposited or otherwise associated with the surface of the working electrode (WE) or reference electrode (RE). In one example, the one or more mediators eliminate or reduce direct oxidation of interfering species that may reach the WE or RE. In one example, the one or more mediators provide a lowering of the operating potential of the WE/RE, for example, from about 0.6V to about 0.3V or less on a platinum electrode, which can reduce or eliminates oxidation of endogenous interfering species. Examples of one or mediators are provided below. Other electrodes, e.g., counter electrodes, can be employed.

In one example, other enzymes or additional components may be added to the polymer mixture(s) that constitute any part of the sensing region to increase the stability of the aforementioned sensor and/or reduce or eliminate the biproducts of the alcohol/alcohol oxidase reaction. Increasing stability includes storage or shelf life and/or operational stability (e.g., retention of enzyme activity during use). For example, byproducts of enzyme reactions may be undesirable for increased shelf life and/or operational stability, and may thus be desirable to reduce or remove. In one example, xanthine oxidase can be used to remove bi-products of one or more enzyme reactions.

In another example, a dehydrogenase enzyme is used with a oxidase for the detection of alcohol alone or in combination with oxygen. Thus, in one example, alcohol dehydrogenase is used to oxidize alcohol to aldehyde in the presence of reduced nicotinamide adenine dinucleotide (NAD(P)H) or reduced nicotinamide adenine dinucleotide phosphate (NAD(P)+). So as to provide a continuous source of NAD(P)H or NAD(P)+, NADH oxidase or NADPH oxidases is used to oxidize the NAD(P)H or NAD(P)+, with the consumption of oxygen. In another example, Diaphorase can be used instead of or in combination with NADH oxidase or NADPH oxidases. Alternatively, an excess amount of NAD(P)H can be incorporated into the one or more enzyme domains and/or the one or more electrodes in an amount so as to accommodate the intended duration of planned life of the sensor.

In the aforementioned dual enzyme configuration, a signal can be sensed either by: (1) an electrically coupled (e.g., “wired”) alcohol dehydrogenase (ADH), for example, using an electro-active hydrogel polymer comprising one or more mediators; or (2) oxygen electrochemical sensing to measure the oxygen consumption of the NADH oxidase. In an alternative example, the co-factor NAD(P)H or NAD(P)+ may be coupled to a polymer, such as dextran, the polymer immobilized in the enzyme domain along with ADH. This provides for retention of the co-factor and availability thereof for the active site of ADH. In the above example, any combination of electrode, interference, resistance, and biointerface membranes can be used to optimize signal, durability, reduce drift, or extend end of use duration. In one example, electrical coupling, for example, directly or indirectly, via a covalent or ionic bond, to at least a portion of a transducing element, such as an aptamer, an enzyme or cofactor and at least a portion of the electrode surface is provided. A chemical moiety capable of assisting with electron transfer from the enzyme or cofactor to the electrode surface can be used and includes one or more mediators as described below.

In one example, any one of the aforementioned continuous alcohol sensor configurations are combined with any one of the aforementioned continuous ketone monitoring configurations to provide a continuous multi-analyte sensor device as further described below. In one example a continuous glucose monitoring configuration combined with any one of the aforementioned continuous alcohol sensor configurations and any one of the aforementioned continuous ketone monitoring configurations to provide a continuous multi-analyte sensor device as further described below.

### Uric Acid Sensor Configurations

In another example, a continuous uric acid sensor device configuration is provided. Thus, in one example, uric acid oxidase (UOX) can be included in one or more enzyme domains and positioned adjacent the working electrode surface. The catalysis of the uric acid using UOX, produces hydrogen peroxide which can be detected using, among other techniques, amperometry, voltametric and impedimetric methods. In one example, to reduce or eliminate the interference from direct oxidation of uric acid on the electrode surface, one or more electrode, interference, and/or resistance domains can be deposited on at least a portion of the working electrode surface. Such membranes can be used to attenuate diffusion of uric acid as well as other analytes to the working electrode that can interfere with signal transduction.

In one alternative example, a uric acid continuous sensing device configuration comprises sensing oxygen level changes about the WE surface, e.g., for example, as in a Clark type electrode setup, or the one or more electrodes can comprise, independently, one or more different polymers such as NAFION™, polyzwitterion polymers, or polymeric mediator adjacent at least a portion of the electrode surface. In one example, the electrode surface with the one or more electrode domains provide for operation at a different or lower voltage to measure oxygen. Oxygen level and its changes in can be sensed, recorded, and correlated to the concentration of uric acid based using, for example, using conventional calibration methods.

In one example, alone or in combination with any of the aforementioned configurations, uric acid sensor configurations, so as to lower the potential at the WE for signal transduction of uric acid, one or more coatings can be deposited on the WE surface. The one or more coatings may be deposited or otherwise formed on the WE surface and/or on other coatings formed thereon using various techniques including, but not limited to, dipping, electrodepositing, vapor deposition, spray coating, etc. In one example, the coated WE surface can provide for redox reactions, e.g., of hydrogen peroxide, at lower potentials (as compared to 0.6 V on platinum electrode surface without such a coating. Example of materials that can be coated or annealed onto the WE surface includes, but are not limited to Prussian Blue, Medola Blue, methylene blue, methylene green, methyl viologen, ferrocyanide, ferrocene, cobalt ion, and cobalt phthalocyanine, and the like.

In one example, one or more secondary enzymes, cofactors and/or mediators (electrically coupled or polymeric mediators) can be added to the enzyme domain with UOX to facilitate direct or indirect electron transfer to the WE. In such configurations, for example, regeneration of the initial oxidized form of secondary enzyme is reduced by the WE for signal transduction. In one example, the secondary enzyme is horse radish peroxidase (HRP).

### Choline Sensor Configurations

In one example continuous choline sensor device can be provided, for example, using choline oxidase enzyme that generates hydrogen peroxide with the oxidation of choline. Thus, in one example, at least one enzyme domain comprises choline oxidase (COX) adjacent at least one WE surface, optionally with one or more electrodes and/or interference membranes positioned in between the WE surface and the at least one enzyme domain. The catalysis of the choline using COX results in creation of hydrogen peroxide which can be detectable using, among other techniques, amperometry, voltametric and impedimetric methods.

In one example, the aforementioned continuous choline sensor configuration is combined with any one of the aforementioned continuous alcohol sensor configurations, and continuous uric acid sensor configurations to provide a continuous multi-analyte sensor device as further described below. This continuous multi-analyte sensor device can further include continuous glucose monitoring capability. Other membranes can be used in the aforementioned continuous choline sensor configuration, such as electrode, resistance, bio-interfacing, and drug releasing membranes.

### Cholesterol Sensor Configurations

In one example, continuous cholesterol sensor configurations can be made using cholesterol oxidase (CHOX), in a manner similar to previously described sensors. Thus, one or more enzyme domains comprising CHOX can be positioned adjacent at least one WE surface. The catalysis of free cholesterol using CHOX results in creation of hydrogen peroxide which can be detectable using, among other techniques, amperometry, voltametric and impedimetric methods.

An exemplary cholesterol sensor configuration using a platinum WE, where at least one interference membrane is positioned adjacent at least one WE surface, over which there is at least one enzyme domain comprising CHOX, over which is positioned at least one resistance domain to control diffusional characteristics was prepared.

The method described above and the cholesterol sensors described can measure free cholesterol, however, with modification, the configuration can measure more types of cholesterol as well as total cholesterol concentration. Measuring different types of cholesterol and total cholesterol is important, since due to low solubility of cholesterol in water significant amount of cholesterol is in unmodified and esterified forms. Thus, in one example, a total cholesterol sample is provided where a secondary enzyme is introduced into the at least one enzyme domain, for example, to provide the combination of cholesterol esterase with CHOX Cholesteryl ester, which essentially represents total cholesterols can be measured indirectly from signals transduced from cholesterol present and formed by the esterase.

In one example, the aforementioned continuous (total) cholesterol sensor configuration is combined with any one of the aforementioned continuous alcohol sensor configurations and/or continuous uric acid sensor configurations to provide a continuous multi-analyte sensor system as further described below. This continuous multi-analyte sensor device can further include continuous glucose monitoring capability. Other membrane configurations can be used in the aforementioned continuous cholesterol sensor configuration, such as one or more electrode domains, resistance domains, bio-interfacing domains, and drug releasing membranes.

### Bilirubin Sensor and Ascorbic Acid Sensor Configurations

In one example, continuous bilirubin and ascorbic acid sensors are provided. These sensors can employ bilirubin oxidase and ascorbate oxidase, respectively. However, unlike some oxidoreductase enzymes, the final product of the catalysis of analytes of bilirubin oxidase and ascorbate oxidase is water instead of hydrogen peroxide. Therefore, redox detection of hydrogen peroxide to correlate with bilirubin or ascorbic acid is not possible. However, these oxidase enzymes still consume oxygen for the catalysis, and the levels of oxygen consumption correlates with the levels of the target analyte present. Thus, bilirubin and ascorbic acid levels can be measured indirectly by electrochemically sensing oxygen level changes, as in a Clark type electrode setup, for example.

Alternatively, a different configuration for sensing bilirubin and ascorbic acid can be employed. For example, an electrode domain including one or more electrode domains comprising electron transfer agents, such as NAFION™, polyzwitterion polymers, or polymeric mediator can be coated on the electrode. Measured oxygen levels transduced from such enzyme domain configurations can be correlated with the concentrations of bilirubin and ascorbic acid levels. In one example, an electrode domain comprising one or more mediators electrically coupled to a working electrode can be employed and correlated to the levels of bilirubin and ascorbic acid levels.

In one example, the aforementioned continuous bilirubin and ascorbic acid sensor configurations can be combined with any one of the aforementioned continuous alcohol sensor configurations, continuous uric acid sensor configurations, continuous cholesterol sensor configurations to provide a continuous multi-analyte sensor device as further described below. This continuous multi-analyte sensor device can further include continuous glucose monitoring capability. Other membranes can be used in the aforementioned continuous bilirubin and ascorbic acid sensor configuration, such as electrode, resistance, bio-interfacing, and drug releasing membranes.

### One-Working-Electrode Configurations for Dual Analyte Detection

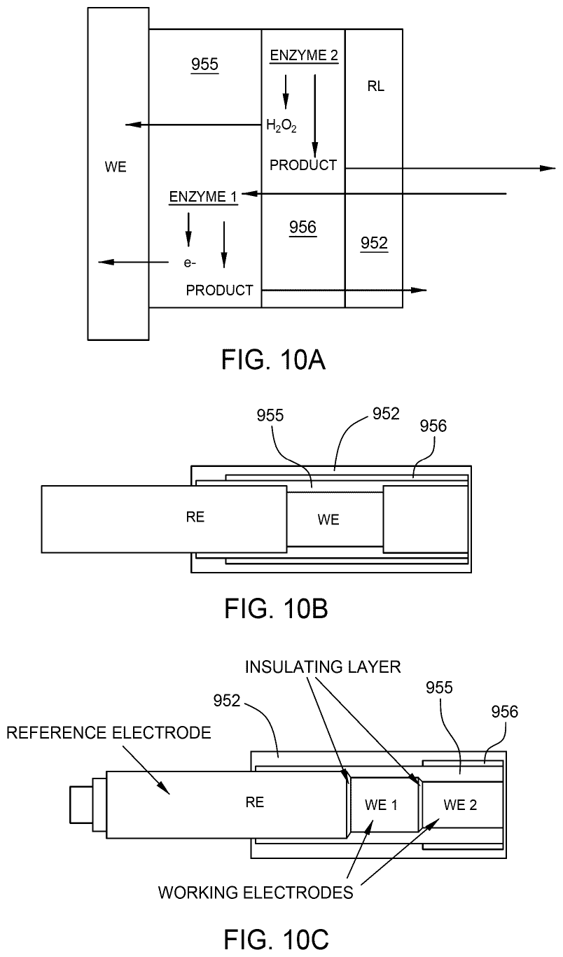

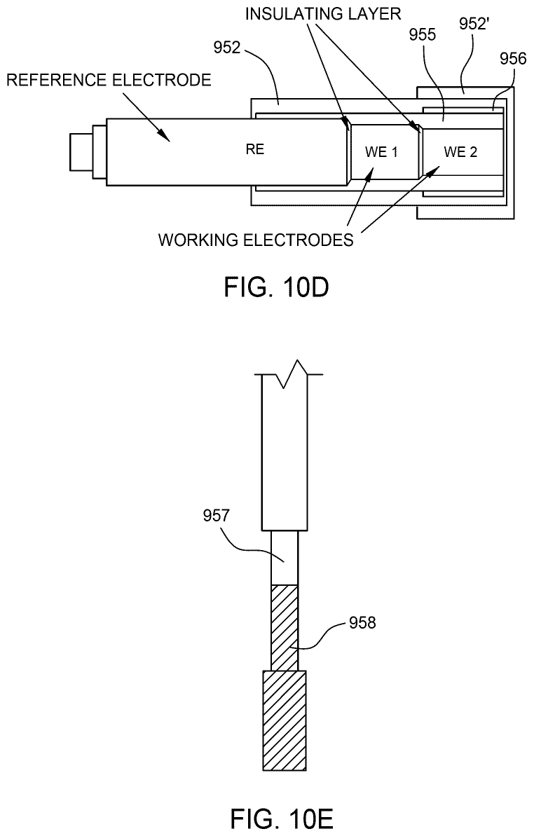

In one example, at least a dual enzyme domain configuration in which each layer contains one or more specific enzymes and optionally one or more cofactors is provided. In a broad sense, one example of a continuous multi-analyte sensor configuration is depicted in FIG. 10A where a first membrane 955 (EZL1) comprising at least one enzyme (Enzyme 1) of the at least two enzyme domain configuration is proximal to at least one surface of a WE. One or more analyte-substrate enzyme pairs with Enzyme 1 transduces at least one detectable signal to the WE surface by direct electron transfer or by mediated electron transfer that corresponds directly or indirectly to an analyte concentration. Second membrane 956 (EZL2) with at least one second enzyme (Enzyme 2) is positioned adjacent 955 EZL1, and is generally more distal from WE than EZL1. One or more resistance domains (RL) 952 can be provided adjacent EZL2 956, and/or between EZL1 955 and EZL2 956. The different enzymes catalyze the transformation of the same analyte, but at least one enzyme in EZL2 956 provides hydrogen peroxide and the other at least one enzyme in EZL1 955 does not provide hydrogen peroxide. Accordingly, each measurable species (e.g., hydrogen peroxide and the other measurable species that is not hydrogen peroxide) generates a signal associated with its concentration.

For example, in the configuration shown in FIG. 10A, a first analyte diffuses through RL 952 and into EZL2 956 resulting in peroxide via interaction with Enzyme 2. Peroxide diffuses at least through EZL1 955 to WE and transduces a signal that corresponds directly or indirectly to the first analyte concentration. A second analyte, which is different from the first analyte, diffuses through RL 952 and EZL2 956 and interacts with Enzyme 1, which results in electron transfer to WE and transduces a signal that corresponds directly or indirectly to the second analyte concentration.

As shown in FIG. 10B, the above configuration is adapted to a conductive wire electrode construct, where at least two different enzyme-containing layers are constructed on the same WE with a single active surface. In one example, the single WE is a wire, with the active surface positioned about the longitudinal axis of the wire. In another example, the single WE is a conductive trace on a substrate, with the active surface positioned about the longitudinal axis of the trace. In one example, the active surface is substantially continuous about a longitudinal axis or a radius.

In the configuration described above, at least two different enzymes can be used and catalyze the transformation of different analytes, with at least one enzyme in EZL2 956 providing hydrogen peroxide and the at least other enzyme in EZL1 955 not providing hydrogen peroxide, e.g., providing electron transfer to the WE surface corresponding directly or indirectly to a concentration of the analyte.

In one example, an inner layer of the at least two enzyme domains EZL1, EZL2 955, 956 comprises at least one immobilized enzyme in combination with at least one mediator that can facilitate lower bias voltage operation of the WE than without the mediator. In one example, for such direct electron transductions, a potential P1 is used. In one example, at least a portion of the inner layer EZL1 955 is more proximal to the WE surface and may have one or more intervening electrode domains and/or overlaying interference and/or bio-interfacing and/or drug releasing membranes, provided that the at least one mediator can facilitate low bias voltage operation with the WE surface. In another example, at least a portion of the inner layer EZL1 955 is directly adjacent the WE.

The second layer of at least dual enzyme domain (the outer layer EZL2 956) of FIG. 10B contains at least one enzyme that result in one or more catalysis reactions that eventually generate an amount of hydrogen peroxide that can electrochemically transduce a signal corresponding to the concentration of the analyte(s). In one example, the generated hydrogen peroxide diffuses through layer EZL2 956 and through the inner layer EZL1 955 to reach the WE surface and undergoes redox at a potential of P2, where P2≠P1. In this way electron transfer and electrolysis (redox) can be selectively controlled by controlling the potentials P1, P2 applied at the same WE surface. Any applied potential durations can be used for P1, P2, for example, equal/periodic durations, staggered durations, random durations, as well as various potentiometric sequences, cyclic voltammetry etc. In some examples, impedimetric sensing may be used. In one example, a phase shift (e.g., a time lag) may result from detecting two signals from two different working electrodes, each signal being generated by a different EZL (EZL1, EZL2, 955, 956) associated with each electrode. The two (or more) signals can be broken down into components to detect the individual signal and signal artifacts generated by each of EZL1 955 and EZL2 956 in response to the detection of two analytes. In some examples, each EZL detects a different analyte. In other examples, both EZLs detect the same analyte.

In another alternative exemplary configuration, as shown in FIGS. 10C-10D a multienzyme domain configuration as described above is provided for a continuous multi-analyte sensor device using a single WE with two or more active surfaces is provided. In one example, the multienzyme domain configurations discussed herein are formed on a planar substrate. In another example, the single WE is coaxial, e.g., configured as a wire, having two or more active surfaces positioned about the longitudinal axis of the wire. Additional wires can be used, for example, as a reference and/or counter electrode. In another example, the single WE is a conductive trace on a substrate, with two or more active surfaces positioned about the longitudinal axis of the trace. At least a portion of the two or more active surfaces are discontinuous, providing for at least two physically separated WE surfaces on the same WE wire or trace. (e.g., WE1, WE2). In one example, the first analyte detected by WE1 is glucose, and the second analyte detected by WE2 is lactate. In another example, the first analyte detected by WE1 is glucose, and the second analyte detected by WE2 is ketones.

Thus, FIGS. 10C-10D depict exemplary configurations of a continuous multi-analyte sensor construct in which EZL1 955, EZL2 956 and RL 952 (resistance domain) as described above, arranged, for example, by sequential dip coating techniques, over a single coaxial wire comprising spatially separated electrode surfaces WE1, WE2. One or more parameters, independently, of the enzyme domains, resistance domains, etc., can be controlled along the longitudinal axis of the WE, for example, thickness, length along the axis from the distal end of the wire, etc. In one example, at least a portion of the spatially separated electrode surfaces are of the same composition. In another example, at least a portion of the spatially separated electrode surfaces are of different composition. In FIGS. 10C-10D, WE1 represents a first working electrode surface configured to operate at P1, for example, and is electrically insulated from second working electrode surface WE2 that is configured to operate at P2, and RE represents a reference electrode RE electrically isolated from both WE1, WE2. One resistance domain is provided in the configuration of FIG. 10C that covers the reference electrode and WE1, WE2. An addition resistance domain is provided in the configuration of FIG. 10D that covers extends over essentially WE2 only. Additional electrodes, such as a counter electrode can be used. Such configurations (whether single wire or dual wire configurations) can also be used to measure the same analyte using two different techniques. Using different signal generating sequences as well as different RLs, the data collected from two different mode of measurements provides increase fidelity, improved performance and device longevity. A non-limiting example is a glucose oxidase (H2O2 producing) and glucose dehydrogenase (electrically coupled) configuration. Measurement of Glucose at two potentials and from two different electrodes provides more data points and accuracy. Such approaches may not be needed for glucose sensing, but the can be applied across the biomarker sensing spectrum of other analytes, alone or in combination with glucoses sensing, such as ketone sensing, ketone/lactate sensing, and ketone/glucose sensing.

In an alternative configuration of that depicted in FIGS. 10C-10D, two or more wire electrodes, which can be colinear, wrapped, or otherwise juxtaposed, are presented, where WE1 is separated from WE2, for example, from other elongated shaped electrode. Insulating layer electrically isolates WE1 from WE2. In this configuration, independent electrode potential can be applied to the corresponding electrode surfaces, where the independent electrode potential can be provided simultaneously, sequentially, or randomly to WE1, WE2. In one example, electrode potentials presented to the corresponding electrode surfaces WES1, WES2, are different. One or more additional electrodes can be present such as a reference electrode and/or a counter electrode. In one example, WES2 is positioned longitudinally distal from WES1 in an elongated arrangement. Using, for example, dip coating methods, WES1 and WES2 are coated with enzyme domain EZL1, while WES2 is coated with different enzyme domain EZL2. Based on the dipping parameters, or different thickness of enzyme domains, multi-layered enzyme domains, each layer independently comprising different loads and/or compositions of enzyme and/or cofactors, mediators can be employed. Likewise, one or more resistance domains (RL) can be applied, each can be of a different thickness along the longitudinal axis of the electrode, and over different electrodes and enzyme domains by controlling dip length and other parameters, for example. With reference to FIG. 10D, such an arrangement of RL's is depicted, where an additional RL 952′ is adjacent WES2 but substantially absent from WES1.

In one example of measuring two different analytes, the above configuration comprising enzyme domain EZL1 955 comprising one or more enzyme(s) and one or more mediators for at least one enzyme of EZL1 to provide for direct electron transfer to the WES1 and determining a concentration of at least a first analyte. In addition, enzyme domain EZL2 956 can comprise at least one enzyme that provides peroxide (e.g., hydrogen peroxide) or consumes oxygen during catalysis with its substrate. The peroxide or the oxygen produced in EZL2 956 migrates to WES2 and provides a detectable signal that corresponds directly or indirectly to a second analyte. For example, WES2 can be carbon, wired to glucose dehydrogenase to measure glucose, while WES1 can be platinum, that measures peroxided produced from lactate oxidase/lactate in EZL2 956. The combinations of electrode material and enzyme(s) as disclosed herein are examples and non-limiting.

In one example, the potentials of P1 and P2 can be separated by an amount of potential so that both signals (from direct electron transfer from EZL1 955 and from hydrogen peroxide redox at WE) can be separately activated and measured. In one example, the electronic module of the sensor can switch between two sensing potentials continuously in a continuous or semi-continuous periodic manner, for example a period (t1) at potential P1, and period (t2) at potential P2 with optionally a rest time with no applied potential. Signal extracted can then be analyzed to measure the concentration of the two different analytes. In another example, the electronic module of the sensor can undergo cyclic voltammetry, providing changes in current when swiping over potentials of P1 and P2 can be correlated to transduced signal coming from either direct electron transfer or electrolysis of hydrogen peroxide, respectably. In one example, the modality of sensing is non-limiting and can include different amperometry techniques, e.g., cyclic voltammetry. In one example, an alternative configuration is provided but hydrogen peroxide production in EZL2 is replaced by another suitable electrolysis compound that maintains the P2≠P1 relationship, such as oxygen, and at least one enzyme-substrate combination that provide the other electrolysis compound.

For example, a continuous multi-analyte sensor configuration, for choline and glucose, in which enzyme domains EZ1 955, EZ2 956 were associated with different WEs, e.g., platinum WE2, and gold WE1 was prepared. In this exemplary case, EZL1 955 contained glucose oxidase and a mediator coupled to WE1 to facilitate electron direct transfer upon catalysis of glucose, and EZL2 956 contained choline oxidase that will catalyze choline and generate hydrogen peroxide for electrolysis at WE2. The EZL's were coated with resistance domains; upon cure and readiness they underwent cyclic voltammetry in the presence of glucose and choline. A wired glucose oxidase enzyme to a gold electrode is capable of transducing signal at 0.2 volts, therefore, by analyzing the current changes at 0.2 volts, the concentration of glucose can be determined. The data also demonstrates that choline concentration is also inferentially detectable at the WE2 platinum electrode if the CV trace is analyzed at the voltage P2.

In one example, either electrode WE1 or WE2 can be, for example, a composite material, for example a gold electrode with platinum ink deposited on top, a carbon/platinum mix, and or traces of carbon on top of platinum, or porous carbon coating on a platinum surface. In one example, with the electrode surfaces containing two distinct materials, for example, carbon used for the wired enzyme and electron transfer, while platinum can be used for hydrogen peroxide redox and detection. As shown in FIG. 10E, an example of such composite electrode surfaces is shown, in which an extended platinum covered wire 957 is half coated with carbon 958, to facilitate multi sensing on two different surfaces of the same electrode. In one example WE2 can be grown on or extend from a portion of the surface or distal end of WE1, for example, by vapor deposition, sputtering, or electrolytic deposition and the like.

Additional examples include a composite electrode material that may be used to form one or both of WE1 and WE2. In one example, a platinum-carbon electrode WE1, comprising EZL1 with glucose dehydrogenase is wired to the carbon surface, and outer EZL2 comprising lactate oxidase generating hydrogen peroxide that is detectable by the platinum surface of the same WE1 electrode. Other examples of this configuration can include ketone sensing (beta-hydroxybutyrate dehydrogenase electrically coupled enzyme in EZL1 955) and glucose sensing (glucose oxidase in EZL2 956). Other membranes can be used in the aforementioned configuration, such as electrode, resistance, bio-interfacing, and drug releasing membranes. In other examples, one or both of the working electrodes (WE1, WE2) may be gold-carbon (Au—C), palladium-carbon (Pd—C), iridium-carbon (Ir—C), rhodium-carbon (Rh—C), or ruthenium-carbon (Ru—C). In some examples, the carbon in the working electrodes discussed herein may instead or additionally include graphene, graphene oxide, or other materials suitable for forming the working electrodes, such as commercially available carbon ink.

### Glycerol Sensor Configurations

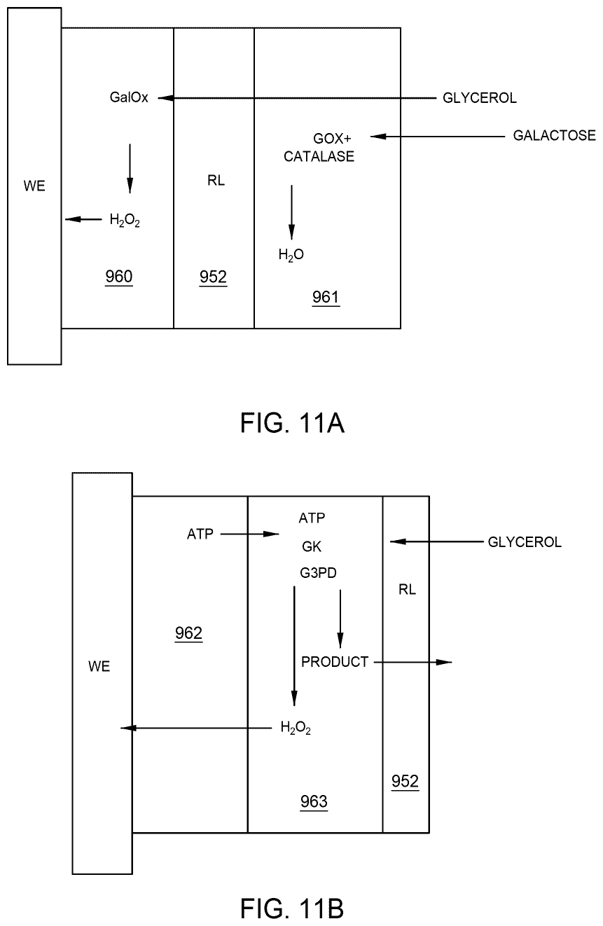

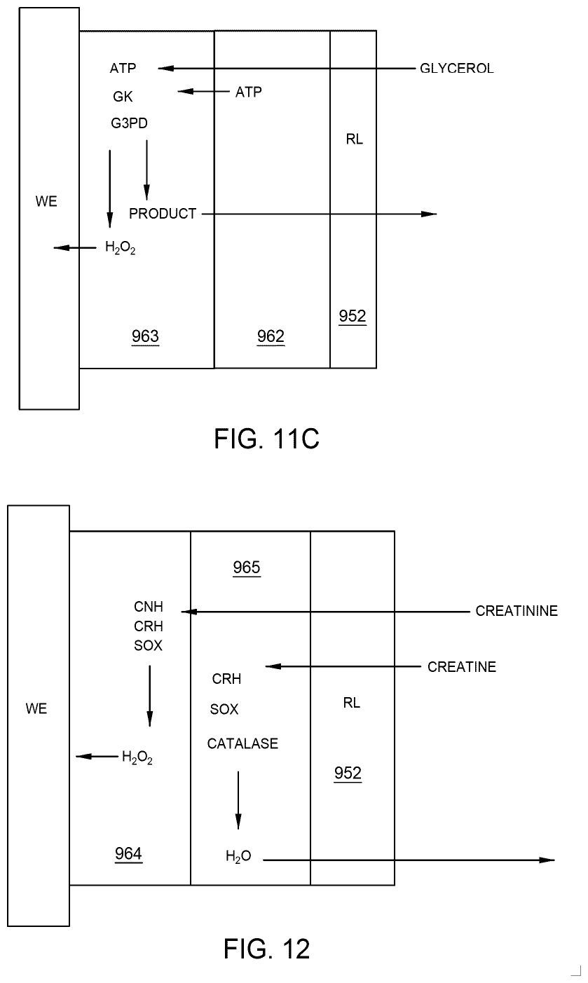

As shown in FIG. 11A, an exemplary continuous glycerol sensor configuration is depicted where a first enzyme domain EZL1 960 comprising galactose oxidase is positioned proximal to at least a portion of a WE surface. A second enzyme domain EZL2 961 comprising glucose oxidase and catalase is positioned more distal from the WE. As shown in FIG. 11A, one or more resistance domains (RL) 952 are positioned between EZL1 960 and EZL2 961. Additional RLs can be employed, for example, adjacent to EZL2 961. Modification of the one or more RL membranes to attenuate the flux of either analyte and increase glycerol to galactose sensitivity ratio is envisaged. The above glycerol sensing configuration provides for a glycerol sensor that can be combined with one or more additional sensor configurations as disclosed herein.

Glycerol can be catalyzed by the enzyme galactose oxidase (GalOx), however, GalOx has an activity ratio of 1%-5% towards glycerol. In one example, the activity of GalOx towards this secondary analyte glycerol can be utilized. The relative concentrations of glycerol in vivo are much higher that galactose (˜2 umol/l for galactose, and ˜100 umol/l for glycerol), which compliments the aforementioned configurations.

If the GalOx present in EZL1 960 membrane is not otherwise functionally limited, then the GalOx will catalyze most if not all of the glycerol that passes through the one or more RLs. The signal contribution from the glycerol present will be higher as compared to the signal contribution from galactose. In one example, the one or more RL's are chemically configured to provide a higher influx of glycerol or a lower influx of galactose.

In another example, a glycol sensor configuration is provided using multiple working electrodes WEs that provides for utilizing signal transduced from both WEs. Utilizing signal transduced from both WEs can provide increasing selectivity. In one example EZL1 960 and EZL2 961 comprise the same oxidase enzyme (e.g., galactose oxidase) with different ratios of enzyme loading, and/or a different immobilizing polymer and/or different number and layers of RL's over the WEs. Such configurations provide for measurement of the same target analyte with different sensitivities, resulting in a dual measurement. Using a mathematical algorithm to correct for noise and interference from a first signal, and inputting the first signal from one sensing electrode with a first analyte sensitivity ratio into the mathematical algorithm, allows for the decoupling of the second signal corresponding to the desired analyte contributions. Modification of the sensitivity ratio of the one or more EZL's to distinguish signals from the interfering species and the analyte(s) of interest can be provided by adjusting one or more of enzyme source, enzyme load in EZL's, chemical nature/diffusional characteristics of EZL's, chemical/diffusional characteristics of the at least one RL's, and combinations thereof.

As discussed herein, a secondary enzyme domain can be utilized to catalyze the non-target analyte(s), reducing their concentration and limiting diffusion towards the sensing electrode through adjacent membranes that contains the primary enzyme and necessary additives. In this example, the most distal enzyme domain, EZL2, 961 is configured to catalyze a non-target analyte that would otherwise react with EZL1, thus providing a potentially less accurate reading of the target analyte (glycerol) concentration. This secondary enzyme domain can act as a “selective diffusion exclusion membrane” by itself, or in some other configurations can be placed above or under a resistant layer (RL) 952. In this example, the target analyte is glycerol and GalOX is used to catalyze glycerol to form a measurable species (e.g., hydrogen peroxide).

In one example, a continuous glycerol sensor configuration is provided using at least glycerol oxidase, which provides hydrogen peroxide upon reaction and catalysis of glycerol. Thus, in one example, enzyme domain comprising glycerol oxidase can be positioned adjacent at least a portion of a WE surface and hydrogen peroxide is detected using amperometry. In another example, enzyme domain comprising glycerol oxidase is used for sensing oxygen level changes, for example, in a Clark type electrode setup. Alternatively, at least a portion of the WE surface can be coated with one more layers of electrically coupled polymers, such as a mediator system discussed below, to provide a coated WE capable of electron transfer from the enzyme at a lower potential. The coated WE can then operate at a different and lower voltage to measure oxygen and its correlation to glycerol concentration.

In another example, a glycerol sensor configuration is provided using glycerol-3-phosphate oxidase in the enzyme domain. In one example, ATP is used as the cofactor. Thus, as shown in FIGS. 11B and 11C, exemplary sensor configurations are depicted where in one example (FIG. 11B), one or more cofactors (e.g. ATP) 962 is proximal to at least a portion of an WE surface. One or more enzyme domains 963 comprising glycerol-3-phospohate oxidase (G3PD), lipase, and/or glycerol kinase (GK) and one or more regenerating enzymes capable of continuously regenerating the cofactor are contained in an enzyme domain are adjacent the cofactor, or more distal from the WE surface than the cofactor layer 962. Examples of regenerating enzymes that can be used to provide ATP regeneration include, but are not limited to, ATP synthase, pyruvate kinase, acetate kinase, and creatine kinase. The one or more regenerating enzymes can be included in one or more enzyme domains, or in a separate layer.

An alternative configuration is shown in FIG. 11C, where one or more enzyme domains 963 comprising G3PD, at least one cofactor and at least one regenerating enzyme, are positioned proximal to at least a portion of WE surface, with one or more cofactor reservoirs 962 adjacent to the enzyme domains comprising G3PD and more distal from the WE surface, and one or more RL's 952 are positioned adjacent the cofactor reservoir. In either of these configurations, an additional enzyme domain comprising lipase can be included to indirectly measure triglyceride, as the lipase will produce glycerol for detection by the aforementioned glycerol sensor configurations.

In another example, a glycerol sensor configuration is provided using dehydrogenase enzymes with cofactors and regenerating enzymes. In one example, cofactors that can be incorporated in the one or more enzyme domains include one or more of NAD(P)H, NADP+, and ATP. In one example, e.g., for use of NAD(P)H a regenerating enzyme can be NADH oxidase or diaphorase to convert NADH, the product of the dehydrogenase catalysis back to NAD(P)H. Similar methodologies can be used for creating other glycerol sensors, for example, glycerol dehydrogenase, combined with NADH oxidase or diaphorase can be configured to measure glycerol or oxygen.

In one example, mathematical modeling can be used to identify and remove interference signals, measuring very low analyte concentrations, signal error and noise reduction so as to improve and increase of multi-analyte sensor end of life. For example, with a two WE electrode configuration where WE1 is coated with a first EZL while WE2 is coated with two or more different EZL, optionally with one or more resistance domains (RL) a mathematical correction such interference can be corrected for, providing for increasing accuracy of the measurements.

Changes of enzyme load, immobilizing polymer and resistance domain characteristics over each analyte sensing region can result in different sensitive ratios between two or more target analyte and interfering species. If the signal are collected and analyzed using mathematical modeling, a more precise concentration of the target analytes can be calculated.

One example in which use of mathematical modeling can be helpful is with glycerol sensing, where galactose oxidase is sensitive towards both galactose and glycerol. The sensitivity ratio of galactose oxidase to glycerol is about is 1%-5% of its sensitivity to galactose. In such case, modification of the sensitivity ratio to the two analytes is possible by adjusting the one or more parameters, such as enzyme source, enzyme load, enzyme domain (EZL) diffusional characteristics, RL diffusional characteristics, and combinations thereof. If two WEs are operating in the sensor system, signal correction and analysis from both WEs using mathematical modeling provides high degree of fidelity and target analyte concentration measurement.

In the above configurations, the proximity to the WE of one or more of these enzyme immobilizing layers discussed herein can be different or reversed, for example if the most proximal to the WE enzyme domain provides hydrogen peroxide, this configuration can be used.

In some examples, the target analyte can be measured using one or multiple of enzyme working in concert. In one example, ATP can be immobilized in one or more EZL membranes, or can be added to an adjacent layer alone or in combination with a secondary cofactor, or can get regenerated/recycled for use in the same EZL or an adjacent third EZL. This configuration can further include a cofactor regenerator enzyme, e.g., alcohol dehydrogenase or NADH oxidase to regenerate NAD(P)H. Other examples of cofactor regenerator enzymes that can be used for ATP regeneration are ATP synthase, pyruvate kinase, acetate kinase, creatine kinase, and the like.

In one example, the aforementioned continuous glycerol sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations to provide a continuous multi-analyte sensor device as further described below. This continuous multi-analyte sensor device can further include continuous glucose monitoring capability. Other configurations can be used in the aforementioned continuous glycerol sensor configuration, such as electrode, resistance, bio-interfacing, and drug releasing membranes.

### Creatinine Sensor Configurations

In one example, continuous creatinine sensor configurations are provided, such configurations containing one or more enzymes and/or cofactors. Creatinine sensor configurations are examples of continuous analyte sensing systems that generate intermediate, interfering products, where these intermediates/interferents are also present in the biological fluids sampled. The present disclosure provides solutions to address these technical problems and provide for accurate, stable, and continuous creatinine monitoring alone or in combination with other continuous multi-analyte sensor configurations.

Creatinine sensors, when in use, are subject to changes of a number of physiologically present intermediate/interfering products, for example sarcosine and creatine, that can affect the correlation of the transduced signal with the creatinine concentration. The physiological concentration range of sarcosine, for example, is an order of magnitude lower that creatinine or creatine, so signal contribution from circulating sarcosine is typically minimal. However, changes in local physiological creatine concentration can affect the creatinine sensor signal. In one example, eliminating or reducing such signal contribution is provided.

Thus, in one example, eliminating or reducing creatine signal contribution of a creatinine sensor comprises using at least one enzyme that will consume the non-targeted interfering analyte, in this case, creatine. For example, two enzyme domains are used, positioned adjacent to each other. At least a portion of a first enzyme domain is positioned proximal to at least a portion of a WE surface, the first enzyme domain comprising one or more enzymes selected from creatinine amidohydrolase (CNH), creatine amidohydrolase (CRH), and sarcosine oxidase (SOX). A second enzyme domain, adjacent the first enzyme domain and more distal from the WE surface, comprises one or more enzymes using creatine as their substrate so as to eliminate or reduce creatine diffusion towards the WE. In one example, combinations of enzymes include CRH, SOX, creatine kinase, and catalase, where the enzyme ratios are tuned to provide ample number of units such that circulating creatine will at least partially be consumed by CRH providing sarcosine and urea, whereas the sarcosine produced will at least partially be consumed by SOX, providing an oxidized form of glycine (e.g. glycine aldehyde) which will at least be partially consumed by catalase. In an alternative configuration of the above, the urea produced by the CRH catalysis can at least partially be consumed by urease to provide ammonia, with the aqueous form (NH4+) being detected via an ion-selective electrode (e.g., nonactin ionophore). Such an alternative potentiometric sensing configuration may provide an alternative to amperometric peroxide detection (e.g., improved sensitivity, limits of detection, and lack of depletion of the reference electrode, alternate pathways/mechanisms). This dual-analyte-sensing example may include a creatinine-potassium sensor having potentiometric sensing at two different working electrodes. In this example, interference signals can be identified and corrected. In one alternative example, the aforementioned configuration can include multi-modal sensing architectures using a combination of amperometry and potentiometry to detect concentrations of peroxide and ammonium ion, measured using amperometry and potentiometry, respectively, and correlated to measure the concentration of the creatinine. In one example, the aforementioned configurations can further comprise one or more configurations (e.g., without enzyme) separating the two enzyme domains to provide complementary or assisting diffusional separations and barriers.

In yet another example, a method to isolate the signal and measure essentially only creatinine is to use a second WE that measures the interfering species (e.g., creatine) and then correct for the signal using mathematical modeling. Thus, for example, signal from the WE interacting with creatine is used as a reference signal. Signal from another WE interacting with creatinine is from corrected for signal from the WE interacting with creatine to selectively determine creatinine concentration.

In yet another example, sensing creatinine is provided by measuring oxygen level changes electrochemically, for example in a Clark type electrode setup, or using one or more electrodes coated with layers of different polymers such as NAFION™ and correlating changes of potential based on oxygen changes, which will indirectly correlate with the concentrations of creatinine.

In yet another example, sensing creatinine is provided by using sarcosine oxidase wired to at least one WE using one or more electrically coupled mediators. In this approach, concentration of creatinine will indirectly correlate with the electron transfer generated signal collected from the WE.

For the aforementioned creatinine sensor configurations based on hydrogen peroxide and/or oxygen measurements the one or more enzymes can be in a single enzyme domain, or the one or more enzymes, independently, can be in one or more enzyme domains, or any other combination thereof, in which in each layer at least one enzyme is present. For the aforementioned creatinine sensor configurations based on use of an electrically coupled sarcosine oxidase containing layer, the layer positioned adjacent to the electrode and is electrically coupled to at least a portion of the electrode surface using mediators.

In another example, the aforementioned creatinine sensor configurations can be sensed using potentiometry by using urease enzyme (UR) that creates ammonium from urea, the urea created by CRH from creatine, the creatine being formed from the interaction of creatinine with CNH. Thus, ammonium can be measured by the above configuration and correlated with the creatinine concentration. Alternatively, creatine amidohydrolase (CI) or creatinine deiminase can be used to create ammonia gas, which under physiological conditions of a transcutaneous sensor, would provide ammonium ion for signal transduction.

In yet another example, sensing creatinine is provided by using one or more enzymes and one or more cofactors. Some non-limiting examples of such configurations include creatinine deaminase (CD) providing ammonium from creatinine, glutamate dehydrogenase (GLDH) providing peroxide from the ammonium, where hydrogen peroxide correlates with levels of present creatinine. The above configuration can further include a third enzyme glutamate oxidase (GLOD) to further break down glutamate formed from the GDLH and create additional hydrogen peroxide. Such combinations of enzymes, independently, can be in one or more enzyme domains, or any other combination thereof, in which in each domain or layer, at least one enzyme is present.

In yet another example, sensing creatinine is provided by the combination of creatinine amidohydrolase (CNH), creatine kinase (CK) and pyruvate kinase (PK), where pyruvate, created by PK can be detected by one or more of either lactate dehydrogenase (LDH) or pyruvate oxidase (POX) enzymes configured independently, where one or more of the aforementioned enzyme are present in one layer, or, in which in each of a plurality of layers comprises at least one enzyme, any other combination thereof.

In such sensor configurations where one or more cofactors and/or regenerating enzymes for the cofactors are used, providing excess amounts of one or more of NADH, NAD(P)H and ATP in any of the one or more configurations can be employed, and one or more diffusion resistance domains can be introduced to limit or prevent flux of the cofactors from their respective membrane(s). Other configurations can be used in the aforementioned configurations, such as electrode, resistance, bio-interfacing, and drug releasing membranes.

In yet another example, creatinine detection is provided by using creatinine deiminase in one or more enzyme domains and providing ammonium to the enzyme domain(s) via catalysis of creatinine. Ammonium ion can then be detected potentiometrically or by using composite electrodes that undergo redox when exposed to ammonium ion, for example NAFION™/polyaniline composite electrodes, in which polyaniline undergoes redox in the presence of ammonium at the electrode under potential. Ammonium concentration can then be correlated to creatinine concentration.

FIG. 12 depicts an exemplary continuous sensor configuration for creatinine. In the example of FIG. 12, the sensor includes a first enzyme domain 964 comprising CNH, CRH, and SOX are adjacent a working electrode WE, e.g., platinum. A second enzyme domain 965 is positioned adjacent the first enzyme domain and is more distal from the WE. One or more resistance domains (RL) 952 can be positioned adjacent the second enzyme domain or between the first and second layers. Creatinine is diffusible through the RL and the second enzyme domain to the first enzyme domain where it is converted to peroxide and transduces a signal corresponding to its concentration. Creatine is diffusible through the RL and is converted in the second enzyme domain to sarcosine and urea, the sarcosine being consumed by the sarcosine oxidase and the peroxide generated is consumed by the catalase, thus preventing transduction of the creatine signal.

For example, variations of the above configuration are possible for continuous monitoring of creatinine alone or in combination with one or more other analytes. Thus, one alternative approach to sensing creatinine could be sensing oxygen level changes electrochemically, for example in a Clark-type electrode setup. In one example, the WE can be coated with layers of different polymers, such as NAFION™ and based on changes of potential oxygen changes, the concentrations of creatinine can be correlated. In yet another example, one or more enzyme most proximal to the WE, i.e., sarcosine oxidase, can be “wired” to the electrode using one or more mediators. Each of the different enzymes in the above configurations can be distributed inside a polymer matrix or domain to provide one enzyme domain. In another example, one or more of the different enzymes discussed herein can be formed as the enzyme domain and can be formed layer by layer, in which each layer has at least one enzyme present. In an example of a “wired” enzyme configuration with a multilayered membrane, the wired enzyme domain would be most proximal to the electrode. One or more interferent layers can be deposited among the multilayer enzyme configuration so as to block of non-targeted analytes from reaching electrodes.

In one example, the aforementioned continuous creatinine sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations, glycerol sensor configurations to provide a continuous multi-analyte sensor device as further described below. This continuous multi-analyte sensor device can further include continuous glucose monitoring capability.

### Lactose Sensor Configurations

In one example, a continuous lactose sensor configuration, alone or in combination with another analyte sensing configuration comprising one or more enzymes and/or cofactors is provided. In a general sense, a lactose sensing configuration using at least one enzyme domain comprising lactase enzyme is used for producing glucose and galactose from the lactose. The produced glucose or galactose is then enzymatically converted to a peroxide for signal transduction at an electrode. Thus, in one example, at least one enzyme domain EZL1 comprising lactase is positioned proximal to at least a portion of a WE surface capable of electrolysis of hydrogen peroxide. In one example, glucose oxidase enzyme (GOX) is included in EZL1, with one or more cofactors or electrically coupled mediators. In another example, galactose oxidase enzyme (GalOx) is included in EZL1, optionally with one or more cofactors or mediators. In one example, glucose oxidase enzyme and galactose oxidase are both included in EZL1. In one example, glucose oxidase enzyme and galactose oxidase are both included in EZL1, optionally with one or more cofactors or electrically coupled mediators.

One or more additional EZL's (e.g. EZL2) can be positioned adjacent the EZL1, where at least a portion of EZL2 is more distal from at least a portion of WE than EZL1. In one example, one or more layers can be positioned in between EZL1 and EZL2, such layers can comprise enzyme, cofactor or mediator or be essentially devoid of one or more of enzymes, cofactors or mediators. In one example, the one or more layers positioned in between EZL1 and EZL2 is essentially devoid of enzyme, e.g., no purposefully added enzyme. In one example one or layers can be positioned adjacent EZL2, being more distal from at least a portion of EZL1 than EZL2, and comprise one or more of the enzymes present in either EZL1 or EZL2.

In one example of the aforementioned lactose sensor configurations, the peroxide generating enzyme can be electrically coupled to the electrode using coupling mediators. The transduced peroxide signals from the aforementioned lactose sensor configurations can be correlated with the level of lactose present.

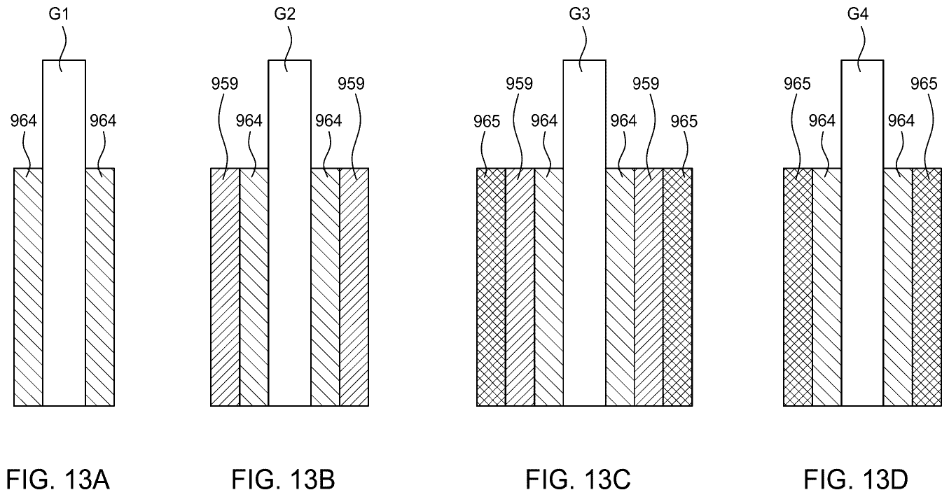

FIG. 13A-13D depict alternative continuous lactose sensor configurations. Thus, in an enzyme domain EZL1 964 most proximal to WE (G1), comprising GalOx and lactase, provides a lactose sensor that is sensitive to galactose and lactose concentration changes and is essentially non-transducing of glucose concentration. As shown in FIGS. 13B-13D, additional layers, including non-enzyme containing layers 959, and a lactase enzyme containing layer 965, and optionally, electrode, resistance, bio-interfacing, and drug releasing membranes. (not shown) are used. Since changes in physiological galactose concentration are minimal, the transduced signal would essentially be from physiological lactose fluctuations.

In one example, the aforementioned continuous lactose sensor configurations can be combined with any one of the aforementioned continuous alcohol sensor configurations, continuous uric acid sensor configurations, continuous cholesterol sensor configurations, continuous bilirubin/ascorbic acid sensor configurations, ketone sensor configurations, choline sensor configurations, glycerol sensor configurations, creatinine sensor configurations to provide a continuous multi-analyte sensor device as further described below. This continuous multi-analyte sensor device can further include continuous glucose monitoring capability. Other membranes can be used in the aforementioned sensor configuration, such as electrode, resistance, bio-interfacing, and drug releasing membranes.

### Urea Sensor Configurations

Similar approach as described above can also be used to create a continuous urea sensor. For example urease (UR), which can break down the urea and to provide ammonium can be used in an enzyme domain configuration. Ammonium can be detected with potentiometry or by using a composite electrodes, e.g., electrodes that undergo redox when exposed to ammonium. Example electrodes for ammonium signal transduction include, but are not limited to, NAFION™/polyaniline composite electrodes, in which polyaniline undergoes redox in the presence of ammonium at an applied potential, with essentially direct correlation of signal to the level of ammonium present in the surrounding. This method can also be used to measure other analytes such as glutamate using the enzyme glutaminase (GLUS).

In one example, the aforementioned continuous uric acid sensor configurations can be combined with any one of the aforementioned continuous alcohol sensor configurations and/or continuous uric acid sensor configurations and/or continuous cholesterol sensor configurations and/or continuous bilirubin/ascorbic acid sensor configurations and/or continuous ketone sensor configurations and/or continuous choline sensor configurations and/or continuous glycerol sensor configurations and/or continuous creatinine sensor configurations and/or continuous lactose sensor configurations to provide a continuous multi-analyte sensor device as further described below. This continuous multi-analyte sensor device can further include continuous glucose monitoring capability. Other membranes can be used in the aforementioned uric acid sensor configuration, such as electrode, resistance, bio-interfacing, and drug releasing membranes.

### Example Embodiments

Implementation examples are described in the following numbered clauses:

Clause 1: A monitoring system, comprising: a continuous analyte sensor configured to penetrate a skin of a patient and generate sensor current indicative of analyte levels of the patient; a sensor electronics module coupled to the continuous analyte sensor, wherein the sensor electronics module comprises: an analog to digital converter configured to: receive the sensor current; and convert the sensor current generated by the continuous analyte sensor into digital signals; one or more processors configured to convert the digital signals to a set of analyte measurements indicative of the analyte levels of the patient; and a Bluetooth antenna configured to transmit the set of analyte measurements wirelessly to a wireless communications device using Bluetooth or BLE communications protocols.

Clause 2: The monitoring system of Clause 1, wherein the sensor electronic module further comprises a sensitivity profile for the monitoring system based on a calibration process performed during manufacturing, wherein one or more processors being configured to convert the digital signals to the set of analyte measurements comprises converting the digital signals to the set of analyte measurements based on the sensitivity profile.

Clause 3: The monitoring system of Clause 1, wherein continuous analyte sensor comprises: a percutaneous wire comprising a proximal portion coupled to the sensor electronics module; and a distal portion comprising a working electrode and a reference electrode, wherein the working electrode is configured to penetrate the skin and extend into a dermis or subcutaneous tissue of the patient.

Clause 4: The monitoring system of Clause 3, wherein the working electrode and the reference electrodes are disposed on a substrate, and the sensor current is at least in part based on a voltage difference generated between the working electrode and the reference electrode.

Clause 5: The monitoring system of any one of Clauses 1-4, wherein the continuous analyte sensor comprises a continuous glucose sensor, and the set of analyte measurements include glucose measurements.

Clause 6: The monitoring system of any one of Clauses 1-5, further comprising one or more memories comprising executable instructions; and one or more processors in data communication with the one or more memories and configured to execute the executable instructions to: determine a classification for the patient based on at least one of: a glucose level of the patient, a glucose baseline of the patient, or a glucose rate of change of the patient derived from the set of analyte measurements of the patient; or input received from the patient, and provide a therapy management recommendation to the patient based on the classification of the patient.

Clause 7: The monitoring system of Clause 6, wherein the one or more processors are further configured to: determine an initial classification for the patient based on the input received from the patient, determine that a confidence score associated with the initial classification is low, and collect the set of analyte measurements of the patient, and the determination of the classification is based on at least one of the glucose level of the patient, the glucose baseline of the patient, or the glucose rate of change of the patient derived from the set of analyte measurements of the patient, and performed in response to the determination that the confidence score associated with the initial classification is low.

Clause 8: The monitoring system of Clause 6, wherein the one or more processors being configured to determine the classification for the patient comprises the one or more processors being configured to determine a healthy patient classification for the patient based on determining that the glucose level of the patient, the glucose baseline of the patient, or the glucose rate of change of the patient are consistent with a population of healthy patients.

Clause 9: The monitoring system of any one of Clauses 6-8, wherein the one or more processors are further configured to: determine a liver disease risk factor based on the glucose level of the patient, the glucose baseline of the patient, or the glucose rate of change of the patient; and provide the liver disease risk factor to the healthy patient.

Clause 10: The monitoring system of any one of Clauses 6-8, wherein the therapy management recommendation provided to the healthy patient comprises a recommendation to avoid nocturnal hypoglycemia, a recommendation to lower glucose level spikes throughout a day, or a recommendation to manage post-prandial glucose dynamics.

Clause 11: The monitoring system of Clause 6, wherein the one or more processors being configured to determine the classification for the patient comprises the one or more processors being configured to determine a liver disease patient classification for the patient based on determining that that the glucose level of the patient, the glucose baseline of the patient, or the glucose rate of change of the patient are consistent with a population of patients with liver disease.

Clause 12: The monitoring system of any one of Clauses 6 or 11, wherein the one or more processors are further configured to: determine a progression of liver disease based on a time to return to baseline glucose level following a meal, an increasing post-prandial glucose area under a curve, a post-prandial glucose spike magnitude, variations in glucose metrics over time, or a glucose response to an exercise session following a meal; and provide an indication of the determined progression of liver disease to the patient.

Clause 13: The monitoring system of any one of Clauses 6, 11, or 12, wherein the one or more processors are further configured to: determine a development of diabetes based on the baseline glucose level, the increasing post-prandial glucose area under a curve, or a presence of a dawn effect derived from the set of glucose measurements; and provide an indication of the determined development of liver disease to the patient.

Clause 14: The monitoring system of any one of Clauses 6, 11, or 12, wherein the therapy management recommendation provided to the patient with liver disease comprises a recommendation to alter meal times, a recommendation to complete an exercise session, a recommendation to avoid evening exercise sessions, a recommendation to avoid alcohol consumption, or a recommendation to begin a medication regimen.

Clause 15: The monitoring system of Clause 6, wherein the one or more processors being configured to determine the classification for the patient comprises the one or more processors being configured to determine a diabetic patient classification for the patient based on determining that that the glucose level of the patient, the glucose baseline of the patient, or the glucose rate of change of the patient are consistent with a population of patients with diabetes.

Clause 16: The monitoring system of any one of Clauses 6 or 15, wherein the one or more processors are further configured to: determine a presence of liver disease based on a post-prandial glucose spike magnitude, a presence of nocturnal hypoglycemia, an increasing post-prandial glucose area under a curve, a glucose level variability, or variations in glucose metrics over time; and provide an indication of the determined presence of liver disease to the patient.

Clause 17: The monitoring system of any one of Clauses 6, 15, or 16, wherein the one or more processors are further configured to: determine a development of liver disease based on the post-prandial glucose spike magnitude, the presence of nocturnal hypoglycemia, the increasing post-prandial glucose area under a curve, the glucose level variability, or variations in glucose metrics over time derived from the set of glucose measurements; and provide an indication of the determined development of liver disease to the patient.

Clause 18: The monitoring system of any one of Clauses 6, 15, 16, or 17, wherein the therapy management recommendation provided to the patient with diabetes comprises a recommendation to alter meal times, a recommendation to avoid alcohol consumption, a recommendation to avoid exercising in an evening, a recommendation to begin a specific medication regimen, or a recommendation to avoid predetermined medications.

Clause 19: A method for providing therapy management recommendations to a patient, comprising: determining a classification for the patient based on at least one of: a glucose level of the patient, a glucose baseline of the patient, or a glucose rate of change of the patient derived from the set of analyte measurements of the patient; or input received from the patient, and providing a therapy management recommendation to the patient based on the classification of the patient.

Clause 20: The method of Clause 19, further comprising determining an initial classification for the patient based on the input received from the patient, determining that a confidence score associated with the initial classification is low, and collecting the set of analyte measurements of the patient, wherein the determination of the classification is: based on at least one of the glucose level of the patient, the glucose baseline of the patient, or the glucose rate of change of the patient derived from the set of analyte measurements of the patient, and performed in response to the determination that the confidence score associated with the initial classification is low.

Clause 21: The method of any one of Clauses 19-20, wherein the determination of the classification for the patient comprises determining a healthy patient classification for the patient based on determining that the glucose level of the patient, the glucose baseline of the patient, or the glucose rate of change of the patient are consistent with a population of healthy patients.

Clause 22: The method of Clause 21, further comprising determining a liver disease risk factor based on the glucose level of the patient, the glucose baseline of the patient, or the glucose rate of change of the patient; and providing the liver disease risk factor to the healthy patient.

Clause 23: The method of any one of Clauses 19-22, wherein the therapy management recommendation provided to the healthy patient comprises a recommendation to avoid nocturnal hypoglycemia, a recommendation to lower glucose level spikes throughout a day, or a recommendation to manage post-prandial glucose dynamics.

Clause 24: The method of any one of Clauses 19-20, wherein the determination of the classification for the patient comprises determining a liver disease patient classification for the patient based on determining that that the glucose level of the patient, the glucose baseline of the patient, or the glucose rate of change of the patient are consistent with a population of patients with liver disease.

Clause 25: The method of Clause 24, further comprising determining a progression of liver disease based on a time to return to baseline glucose level following a meal, an increasing post-prandial glucose area under a curve, a post-prandial glucose spike magnitude, variations in glucose metrics over time, or a glucose response to an exercise session following a meal; and providing an indication of the determined progression of liver disease to the patient.

Clause 26: The method of Clause 24, further comprising determining a development of diabetes based on the baseline glucose level, the increasing post-prandial glucose area under a curve, or a presence of a dawn effect derived from the set of glucose measurements; and providing an indication of the determined development of liver disease to the patient.

Clause 27: The method of any one of Clauses 19 or 24-26, wherein the therapy management recommendation provided to the patient with liver disease comprises a recommendation to alter meal times, a recommendation to complete an exercise session, a recommendation to avoid evening exercise sessions, a recommendation to avoid alcohol consumption, or a recommendation to begin a medication regimen.

Clause 28: The method of any one of Clauses 19-20, wherein the determination of the classification for the patient comprises determining a diabetic patient classification for the patient based on determining that that the glucose level of the patient, the glucose baseline of the patient, or the glucose rate of change of the patient are consistent with a population of patients with diabetes.

Clause 29: The method of Clause 28, further comprising determining a presence of liver disease based on a post-prandial glucose spike magnitude, a presence of nocturnal hypoglycemia, an increasing post-prandial glucose area under a curve, a glucose level variability, or variations in glucose metrics over time; and providing an indication of the determined presence of liver disease to the patient.

Clause 30: The method of Clause 28, further comprising determining a development of liver disease based on the post-prandial glucose spike magnitude, the presence of nocturnal hypoglycemia, the increasing post-prandial glucose area under a curve, the glucose level variability, or variations in glucose metrics over time derived from the set of glucose measurements; and providing an indication of the determined development of liver disease to the patient.

Clause 31: The method of any one of Clauses 19-20 or 28-30, wherein the therapy management recommendation provided to the patient with diabetes comprises a recommendation to alter meal times, a recommendation to avoid alcohol consumption, a recommendation to avoid exercising in an evening, a recommendation to begin a specific medication regimen, or a recommendation to avoid predetermined medications.

Clause 32: A non-transitory computer readable medium comprising instructions that when executed by one or more processors, cause the one or more processors to perform a method for providing therapy management recommendations to a patient, the method comprising: determining a classification for the patient based on at least one of: a glucose level of the patient, a glucose baseline of the patient, or a glucose rate of change of the patient derived from the set of analyte measurements of the patient; or input received from the patient, and providing a therapy management recommendation to the patient based on the classification of the patient.

Clause 33: The medium of Clause 32, further comprising determining an initial classification for the patient based on the input received from the patient, determining that a confidence score associated with the initial classification is low, and collecting the set of analyte measurements of the patient, wherein the determination of the classification is: based on at least one of the glucose level of the patient, the glucose baseline of the patient, or the glucose rate of change of the patient derived from the set of analyte measurements of the patient, and performed in response to the determination that the confidence score associated with the initial classification is low.

Clause 34: The medium of any one of Clauses 32-33, wherein the determination of the classification for the patient comprises determining a healthy patient classification for the patient based on determining that the glucose level of the patient, the glucose baseline of the patient, or the glucose rate of change of the patient are consistent with a population of healthy patients.

Clause 35: The medium of Clause 34, further comprising determining a liver disease risk factor based on the glucose level of the patient, the glucose baseline of the patient, or the glucose rate of change of the patient; and providing the liver disease risk factor to the healthy patient.

Clause 36: The medium of any one of Clauses 32-35, wherein the therapy management recommendation provided to the healthy patient comprises a recommendation to avoid nocturnal hypoglycemia, a recommendation to lower glucose level spikes throughout a day, or a recommendation to manage post-prandial glucose dynamics.

Clause 37: The medium of any one of Clauses 32-33, wherein the determination of the classification for the patient comprises determining a liver disease patient classification for the patient based on determining that that the glucose level of the patient, the glucose baseline of the patient, or the glucose rate of change of the patient are consistent with a population of patients with liver disease.

Clause 38: The medium of Clause 37, further comprising determining a progression of liver disease based on a time to return to baseline glucose level following a meal, an increasing post-prandial glucose area under a curve, a post-prandial glucose spike magnitude, variations in glucose metrics over time, or a glucose response to an exercise session following a meal; and providing an indication of the determined progression of liver disease to the patient.

Clause 39: The medium of Clause 37, further comprising determining a development of diabetes based on the baseline glucose level, the increasing post-prandial glucose area under a curve, or a presence of a dawn effect derived from the set of glucose measurements; and providing an indication of the determined development of liver disease to the patient.

Clause 40: The medium of any one of Clauses 32 or 37-39, wherein the therapy management recommendation provided to the patient with liver disease comprises a recommendation to alter meal times, a recommendation to complete an exercise session, a recommendation to avoid evening exercise sessions, a recommendation to avoid alcohol consumption, or a recommendation to begin a medication regimen.

Clause 41: The medium of any one of Clauses 32-33, wherein the determination of the classification for the patient comprises determining a diabetic patient classification for the patient based on determining that that the glucose level of the patient, the glucose baseline of the patient, or the glucose rate of change of the patient are consistent with a population of patients with diabetes.

Clause 42: The medium of Clause 41, further comprising determining a presence of liver disease based on a post-prandial glucose spike magnitude, a presence of nocturnal hypoglycemia, an increasing post-prandial glucose area under a curve, a glucose level variability, or variations in glucose metrics over time; and providing an indication of the determined presence of liver disease to the patient.

Clause 43: The medium of Clause 41, further comprising determining a development of liver disease based on the post-prandial glucose spike magnitude, the presence of nocturnal hypoglycemia, the increasing post-prandial glucose area under a curve, the glucose level variability, or variations in glucose metrics over time derived from the set of glucose measurements; and providing an indication of the determined development of liver disease to the patient.

Clause 44: The medium of any one of Clauses 32-33 or 41-43, wherein the therapy management recommendation provided to the patient with diabetes comprises a recommendation to alter meal times, a recommendation to avoid alcohol consumption, a recommendation to avoid exercising in an evening, a recommendation to begin a specific medication regimen, or a recommendation to avoid predetermined medications.

Clause 45: The method of Clause 19, wherein the classification is one of a healthy patient classification, a liver disease patient classification, or a diabetic patient classification.

Clause 46: The method of Clause 19, further comprising: generating or obtaining a second set of analyte measurements of the patient subsequent to providing the therapy management recommendation.

Clause 47: The method of Claus 19, further comprising: monitoring analyte data of the patient subsequent to providing the therapy management recommendation, the analyte data including a second set of analyte measurements.

Clause 48: A method of monitoring a disease state of a patient, comprising: receiving analyte data associated with a host; determining a disease state of the host based on the received analyte data; determining a first set of therapy managements recommendations based on the disease state of the host and the real time analyte measurements of the host; providing the therapy management recommendations to the host; monitoring the user analye data over a second time interval after providing the recommendation; determining a second disease state of the host based on the monitoring; and

Clause 49: The method of Clause 48, further comprising: providing one or more therapy management recommendations to the host based on the new disease state of the host.

Clause 50: A method of monitoring a disease state of a host, comprising: determining a first classification of the host; monitoring for one or more analyte characteristics associated with the classification; determining a disease state metric for the host based on the monitoring; providing an output of the disease state metric to the host.

### Additional Considerations

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof, adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “comprising as used herein is synonymous with “including.” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples and examples described herein, but rather to also cover all modification and alternatives coming with the true scope and spirit of the invention.

1. A monitoring system, comprising:
a continuous analyte sensor configured to penetrate a skin of a patient and generate sensor current indicative of analyte levels of the patient;
a sensor electronics module coupled to the continuous analyte sensor, wherein the sensor electronics module comprises:
an analog to digital converter configured to:
receive the sensor current; and
convert the sensor current generated by the continuous analyte sensor into digital signals;
one or more processors configured to convert the digital signals to a set of analyte measurements indicative of the analyte levels of the patient; and
a Bluetooth antenna configured to transmit the set of analyte measurements wirelessly to a wireless communications device using Bluetooth or BLE communications protocols.

2. The monitoring system of claim 1, wherein the sensor electronic module further comprises a sensitivity profile for the monitoring system based on a calibration process performed during manufacturing, wherein one or more processors being configured to convert the digital signals to the set of analyte measurements comprises converting the digital signals to the set of analyte measurements based on the sensitivity profile.

3. The monitoring system of claim 1, wherein continuous analyte sensor comprises:
a percutaneous wire comprising:
a proximal portion coupled to the sensor electronics module; and
a distal portion comprising a working electrode and a reference electrode, wherein the working electrode is configured to penetrate the skin and extend into a dermis or subcutaneous tissue of the patient.

4. The monitoring system of claim 3, wherein:
the working electrode and the reference electrodes are disposed on a substrate, and
the sensor current is at least in part based on a voltage difference generated between the working electrode and the reference electrode.

5. The monitoring system of claim 1, wherein:
the continuous analyte sensor comprises a continuous glucose sensor, and
the set of analyte measurements include glucose measurements.

6. The monitoring system of claim 5, further comprising:
one or more memories comprising executable instructions; and
one or more processors in data communication with the one or more memories and configured to execute the executable instructions to:
determine a classification for the patient based on at least one of:
a glucose level of the patient, a glucose baseline of the patient, or a glucose rate of change of the patient derived from the set of analyte measurements of the patient; or
input received from the patient, and
provide a therapy management recommendation to the patient based on the classification of the patient.

7. The monitoring system of claim 6, wherein:
the one or more processors are further configured to:
determine an initial classification for the patient based on the input received from the patient,
determine that a confidence score associated with the initial classification is low, and
collect the set of analyte measurements of the patient, and
the determination of the classification is:
based on at least one of the glucose level of the patient, the glucose baseline of the patient, or the glucose rate of change of the patient derived from the set of analyte measurements of the patient, and
performed in response to the determination that the confidence score associated with the initial classification is low.

8. The monitoring system of claim 6, wherein the one or more processors being configured to determine the classification for the patient comprises the one or more processors being configured to determine a healthy patient classification for the patient based on determining that the glucose level of the patient, the glucose baseline of the patient, or the glucose rate of change of the patient are consistent with a population of healthy patients.

9. The monitoring system of claim 8, wherein the one or more processors are further configured to:
determine a liver disease risk factor based on the glucose level of the patient, the glucose baseline of the patient, or the glucose rate of change of the patient; and
provide the liver disease risk factor to the healthy patient.

10. The monitoring system of claim 8, wherein the therapy management recommendation provided to the healthy patient comprises a recommendation to avoid nocturnal hypoglycemia, a recommendation to lower glucose level spikes throughout a day, or a recommendation to manage post-prandial glucose dynamics.

11. The monitoring system of claim 6, wherein the one or more processors being configured to determine the classification for the patient comprises the one or more processors being configured to determine a liver disease patient classification for the patient based on determining that that the glucose level of the patient, the glucose baseline of the patient, or the glucose rate of change of the patient are consistent with a population of patients with liver disease.

12. The monitoring system of claim 11, wherein the one or more processors are further configured to:
determine a progression of liver disease based on a time to return to baseline glucose level following a meal, an increasing post-prandial glucose area under a curve, a post-prandial glucose spike magnitude, variations in glucose metrics over time, or a glucose response to an exercise session following a meal; and
provide an indication of the determined progression of liver disease to the patient.

13. The monitoring system of claim 12, wherein the one or more processors are further configured to:
determine a development of diabetes based on the baseline glucose level, the increasing post-prandial glucose area under a curve, or a presence of a dawn effect derived from the set of glucose measurements; and
provide an indication of the determined development of liver disease to the patient.

14. The monitoring system of claim 11, wherein the therapy management recommendation provided to the patient with liver disease comprises a recommendation to alter meal times, a recommendation to complete an exercise session, a recommendation to avoid evening exercise sessions, a recommendation to avoid alcohol consumption, or a recommendation to begin a medication regimen.

15. The monitoring system of claim 6, wherein the one or more processors being configured to determine the classification for the patient comprises the one or more processors being configured to determine a diabetic patient classification for the patient based on determining that that the glucose level of the patient, the glucose baseline of the patient, or the glucose rate of change of the patient are consistent with a population of patients with diabetes.

16. The monitoring system of claim 15, wherein the one or more processors are further configured to:
determine a presence of liver disease based on a post-prandial glucose spike magnitude, a presence of nocturnal hypoglycemia, an increasing post-prandial glucose area under a curve, a glucose level variability, or variations in glucose metrics over time; and
provide an indication of the determined presence of liver disease to the patient.

17. The monitoring system of claim 16, wherein the one or more processors are further configured to:
determine a development of liver disease based on the post-prandial glucose spike magnitude, the presence of nocturnal hypoglycemia, the increasing post-prandial glucose area under a curve, the glucose level variability, or variations in glucose metrics over time derived from the set of glucose measurements; and
provide an indication of the determined development of liver disease to the patient.

18. The monitoring system of claim 15, wherein the therapy management recommendation provided to the patient with diabetes comprises a recommendation to alter meal times, a recommendation to avoid alcohol consumption, a recommendation to avoid exercising in an evening, a recommendation to begin a specific medication regimen, or a recommendation to avoid predetermined medications.
